(19) United States
(12) Patent Application Publication
(10) Pub. No.: US 2025/0073285 A1
(43) Pub. Date: Mar. 6, 2025

(54) PHARMACEUTICAL COMPOSITION CONTAINING BACTERIA

(71) Applicant: Evelo Biosciences, Inc., Cambridge, MA (US)

(72) Inventors: Mehmedalija Jahic, Waltham, MA (US); Collin McKenna, North Reading, MA (US)

(21) Appl. No.: 18/285,890

(22) PCT Filed: Apr. 8, 2022
(86) PCT No.: PCT/US22/23979

§ 371 Date: Oct. 6, 2023

Related U.S. Application Data
(60) Provisional application No. 63/197,000, filed on Jun. 4, 2021.
(60) Provisional application No. 63/172,370, filed on Apr. 8, 2021.

(51) Int. Cl. A61K 35/747; A61K 9/14; A61K 9/19; A61K 9/48; A61K 35/745; A61K 45/06; A61K 47/18; A61K 47/26; A61K 47/36

(52) U.S. Cl. CPC A61K 35/747; A61K 9/145; A61K 9/19; A61K 9/4858; A61K 9/4891; A61K 35/745; A61K 45/06; A61K 47/183; A61K 47/26; A61K 47/36

(57) ABSTRACT

Methods and compositions related to pharmaceutical agents containing bacteria are provided herein.

### CROSS REFERENCE TO RELATED APPLICATIONS

This application claims the benefit of the following U.S. Provisional Application Nos.: 63/172,370, filed Apr. 8, 2021, and 63/197,000 filed Jun. 4, 2021, and the entire contents of each of which are incorporated herein by reference.

### SUMMARY

In certain aspects, provided herein are pharmaceutical agents (e.g., powders) comprising bacteria (e.g., freeze dried bacteria), wherein the bacteria in the pharmaceutical agent are present at a total cell count (TCC) of at least 1×10¹¹ cells/gram of the pharmaceutical agent. In some embodiments, the bacteria are present in the pharmaceutical agent at a total cell count (TCC) of at least 3.3×10¹¹ cells/gram of the pharmaceutical agent. In some embodiments, the bacteria are present in the pharmaceutical agent at a total cell count (TCC) of at least 5×10¹¹ cells/gram of the pharmaceutical agent. In some embodiments, the bacteria are present in the pharmaceutical agent at a total cell count (TCC) of at least 7×10¹¹ cells/gram of the pharmaceutical agent. In some embodiments, the bacteria are present in the pharmaceutical agent at a total cell count (TCC) of at least 1×10¹² cells/gram of the pharmaceutical agent. In some embodiments, the bacteria are present in the pharmaceutical agent at a total cell count (TCC) of at least 2×10¹² cells/gram of the pharmaceutical agent. In some embodiments, the bacteria are present in the pharmaceutical agent at a total cell count (TCC) of between about 1×10¹¹ cells/gram to about 2.5×10¹² cells/gram of the pharmaceutical agent. In some embodiments, the bacteria are present in the pharmaceutical agent at a total cell count (TCC) of between about 3.3×10¹¹ cells/gram to about 2.5×10¹² cells/gram of the pharmaceutical agent. In some embodiments, the bacteria are present in the pharmaceutical agent at a total cell count (TCC) of between about 5×10¹¹ cells/gram to about 2.5×10¹² cells/gram of the pharmaceutical agent. In some embodiments, the bacteria are present in the pharmaceutical agent at a total cell count (TCC) of between about 7×10¹¹ cells/gram to about 2.4×10¹² cells/gram of the pharmaceutical agent. In some embodiments, the bacteria are present in the pharmaceutical agent at a total cell count (TCC) of about 2×10¹² cells/gram of the pharmaceutical agent. In some embodiments, the bacteria are present in the pharmaceutical agent at a total cell count (TCC) of between about 1×10¹² cells/gram to about 2×10¹² cells/gram of the pharmaceutical agent. In some embodiments, the bacteria are present in the pharmaceutical agent at a total cell count (TCC) of between about 1.3×10¹² cells/gram to about 2.4×10¹² cells/gram of the pharmaceutical agent. In some embodiments, the bacteria are present in the pharmaceutical agent at a total cell count (TCC) of about 2.5×10¹² cells/gram of the pharmaceutical agent. In some embodiments, the bacteria are present in the pharmaceutical agent at a total cell count (TCC) of about 1.2×10¹² cells/gram of the pharmaceutical agent. In some embodiments, the bacteria are present in the pharmaceutical agent at a total cell count (TCC) of about 1.5×10¹² cells/gram of the pharmaceutical agent. In some embodiments, the bacteria are present in the pharmaceutical agent at a total cell count (TCC) of about 6×10¹¹ cells/gram of the pharmaceutical agent.

In certain aspects, provided herein are pharmaceutical agents (e.g., powders) comprising bacteria (e.g., freeze dried bacteria) and a cryoprotectant.

In some embodiments, the cryoprotectant comprises sucrose. In some embodiments, the cryoprotectant comprises dextran. In some embodiments, the cryoprotectant comprises sucrose and dextran. In some embodiments, the cryoprotectant comprises sucrose and dextran in equivalent amounts (e.g., on a percent weight by weight basis). In some embodiments, the cryoprotectant comprises sucrose, and dextran. In some embodiments, the cryoprotectant comprises sucrose, dextran, and L-cysteine HCl (e.g., a form of L-cysteine). In some embodiments, the cryoprotectant does not comprise L-cysteine HCl.

In some embodiments, the pharmaceutical agent comprises about 6% to about 12% (weight/weight) sucrose. In some embodiments, the pharmaceutical agent comprises about 8% to about 12% (weight/weight) sucrose. In some embodiments, the pharmaceutical agent comprises about 11% (weight/weight) sucrose. In some embodiments, the pharmaceutical agent comprises about 6% to about 12% (weight/weight) dextran. In some embodiments, the pharmaceutical agent comprises about 8% to about 12% (weight/weight) dextran. In some embodiments, the pharmaceutical agent comprises about 11% (weight/weight) dextran. In some embodiments, the pharmaceutical agent comprises about 6% to about 12% (weight/weight) sucrose and about 6% to about 12% (weight/weight) dextran. In some embodiments, the pharmaceutical agent comprises about 8% to about 12% (weight/weight) sucrose and about 8% to about 12% (weight/weight) dextran. In some embodiments, the pharmaceutical agent comprises about 11% (weight/weight) sucrose and about 11% (weight/weight) dextran. In some embodiments, the pharmaceutical agent comprises about 0.1% (weight/weight) L-cysteine HCl. In some embodiments, the pharmaceutical agent comprises about 0.1% to about 0.3% (weight/weight) L-cysteine HCl. In some embodiments, the pharmaceutical agent comprises about 0.15% to about 0.25% (weight/weight) L-cysteine HCl. In some embodiments, the pharmaceutical agent comprises about 10% to about 0.25% or about 0.15% to about 0.35% (weight/weight) L-cysteine HCl.

In some embodiments, the pharmaceutical agent comprises about 7% to about 21% (weight/weight) sucrose. In some embodiments, the pharmaceutical agent comprises about 10% to about 19% (weight/weight) sucrose. In some embodiments, the pharmaceutical agent comprises about 15% (weight/weight) sucrose. In some embodiments, the pharmaceutical agent comprises about 7% to about 21% (weight/weight) dextran. In some embodiments, the pharmaceutical agent comprises about 10% to about 19% (weight/weight) dextran. In some embodiments, the pharmaceutical agent comprises about 15% (weight/weight) dextran. In some embodiments, the pharmaceutical agent comprises about 7% to about 21% (weight/weight) sucrose and about 7% to about 21% (weight/weight) dextran. In some embodiments, the pharmaceutical agent comprises about 10% to about 19% (weight/weight) sucrose and about 10% to about 19% (weight/weight) dextran. In some embodiments, the pharmaceutical agent comprises about 15% (weight/weight) sucrose and about 15% (weight/weight) dextran. In some embodiments, the pharmaceutical agent comprises about 0.01% (weight/weight) L-cysteine HCl. In some embodiments, the pharmaceutical agent comprises about 0.1% to about 0.4% (weight/weight) L-cysteine HCl. In some embodiments, the pharmaceutical agent comprises about 0.15% to about 0.35% (weight/weight) L-cysteine HCl.

In some embodiments, the cryoprotectant comprises a dried (dehydrated) maize glucose syrup (such as Glucidex), such as a dried maize glucose syrup with a dextrose equivalent (DE) of 21 (such as Glucidex 21). In some embodiments, the cryoprotectant comprises sucrose. In some embodiments, the cryoprotectant comprises dextrose (also referred to as glucose). In some embodiments, the cryoprotectant comprises monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and dextrose. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, and monosodium glutamate. In some embodiments, the cryoprotectant comprises dextrose and monosodium glutamate in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, monosodium glutamate, and L-cysteine HCl (e.g., a form of L-cysteine). In some embodiments, the cryoprotectant does not comprise L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 26% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 21% to about 29% (weight/weight) sucrose. In some embodiments, the pharmaceutical agent comprises about 23% to about 27% (weight/weight) sucrose. In some embodiments, the pharmaceutical agent comprises about 25% (weight/weight) sucrose. In some embodiments, the pharmaceutical agent comprises about 6% to about 11% (weight/weight) dextrose. In some embodiments, the pharmaceutical agent comprises about 7% to about 10% (weight/weight) dextrose. In some embodiments, the pharmaceutical agent comprises about 9% (weight/weight) dextrose. In some embodiments, the pharmaceutical agent comprises about 4% to about 10% (weight/weight) glutamate. In some embodiments, the pharmaceutical agent comprises about 5% to about 9% (weight/weight) glutamate. In some embodiments, the pharmaceutical agent comprises about 7% (weight/weight) glutamate.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21) and about 21% to about 29% (weight/weight) sucrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21) and about 23% to about 27% (weight/weight) sucrose. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 6% to about 11% (weight/weight) dextrose. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 4% to about 10% (weight/weight) glutamate. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; about 6% to about 11% (weight/weight) dextrose; and about 4% to about 10% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); about 23% to about 27% (weight/weight) sucrose; and about 7% to about 10% (weight/weight) dextrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; and about 5% to about 9% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; about 7% to about 1% (weight/weight) dextrose; and about 5% to about 9% (weight/weight) glutamate. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In certain embodiments, the pharmaceutical agent comprises about 40% to about 75% (weight/weight) bacteria (e.g., freeze dried bacteria). In certain embodiments, the pharmaceutical agent comprises about 35% to about 70% (weight/weight) bacteria (e.g., freeze dried bacteria). In certain embodiments, the pharmaceutical agent comprises about 64% (weight/weight) bacteria (e.g., freeze dried bacteria).

In certain embodiments, the pharmaceutical agent comprises about 15% to about 35% (weight/weight) bacteria (e.g., freeze dried bacteria). In certain embodiments, the pharmaceutical agent comprises about 18% to about 30% (weight/weight) bacteria (e.g., freeze dried bacteria). In certain embodiments, the pharmaceutical agent comprises about 25% (weight/weight) bacteria (e.g., freeze dried bacteria).

In certain embodiments, the pharmaceutical agent comprises Prevotella bacteria.

In certain embodiments, the pharmaceutical agent comprises Veillonella bacteria.

In some embodiments, the pharmaceutical agent has a fine and smooth granulated powder appearance.

In some embodiments, the pharmaceutical agent has an off-white to brown, fine powder appearance.

In some aspects, the disclosure provides a pharmaceutical composition that comprises a pharmaceutical agent (e.g., powder) described herein and one or more excipients.

In some aspects, the disclosure provides a method comprising combining bacteria (e.g., a pellet comprising bacteria) with a cryoprotectant solution, thereby preparing a formulated paste. In some embodiments, the disclosure provides a formulated paste prepared by this method.

In some embodiments, the method further comprises freeze drying the formulated paste, to thereby prepare a freeze-dried product. In some embodiments, the freeze drying comprises primary drying. In some embodiments, the freeze drying comprises primary drying and secondary drying. In some embodiments, the disclosure provides a freeze-dried product prepared by this method.

In some embodiments, the method further comprises milling the freeze-dried product, to thereby prepare a freeze-dried powder (e.g., powder, e.g., pharmaceutical agent). In some embodiments, the disclosure provides a pharmaceutical agent prepared by this method.

In some embodiments, the method further comprises combining the freeze-dried powder with one or more excipients to thereby prepare a pharmaceutical composition. In some embodiments, the disclosure provides a pharmaceutical composition prepared by this method.

In some embodiments, the cryoprotectant solution is mixed with the pellet in a ratio of about 0.2 to about 0.5 gram (g) cryoprotectant solution per gram of pellet; about 0.05 to about 0.25 gram (g) cryoprotectant solution per gram of pellet; about 0.06 to about 0.1 gram (g) cryoprotectant solution per gram of pellet; or about 0.15 to about 0.2 gram (g) cryoprotectant solution per gram of pellet. In some embodiments, the cryoprotectant solution is mixed with the pellet in a ratio of about 0.4 gram (g) cryoprotectant solution per gram of pellet. In some embodiments, the cryoprotectant solution is mixed with the pellet in a ratio of about 0.18 gram (g) cryoprotectant solution per gram of pellet. In some embodiments, the cryoprotectant solution is mixed with the pellet in a ratio of about 0.1 (e.g., 0.08) gram (g) of cryoprotectant solution per gram of pellet.

In some embodiments, the cryoprotectant solution is mixed with the pellet at a ratio of 4% to 10% (volume/volume), e.g., 5% to 8% (volume/volume). In some embodiments, the cryoprotectant solution is mixed with the pellet at a ratio of about 6.5% (volume/volume).

In some embodiments, the cryoprotectant solution comprises sucrose. In some embodiments, the cryoprotectant solution comprises dextran. In some embodiments, the cryoprotectant solution comprises sucrose and dextran. In some embodiments, the cryoprotectant solution comprises sucrose and dextran in equivalent amounts (e.g., on a percent weight by weight basis). In some embodiments, the cryoprotectant solution comprises sucrose, dextran, and L-cysteine HCl (e.g., a form of L-cysteine). In some embodiments, the cryoprotectant solution does not comprise L-cysteine HCl.

In some embodiments, the cryoprotectant solution comprises about 10% to about 30% (weight/weight) sucrose. In some embodiments, the cryoprotectant solution comprises about 15% to about 35% (weight/weight) sucrose. In some embodiments, the cryoprotectant solution comprises about 20% (weight/weight) sucrose.

In some embodiments, the cryoprotectant solution comprises about 10% to about 30% (weight/weight) dextran. In some embodiments, the cryoprotectant solution comprises about 15% to about 35% (weight/weight) dextran. In some embodiments, the cryoprotectant solution comprises about 20% (weight/weight) dextran.

In some embodiments, the cryoprotectant solution comprises about 10% to about 30% (weight/weight) sucrose and about 10% to about 30% (weight/weight) dextran. In some embodiments, the cryoprotectant solution comprises about 15% to about 35% (weight/weight) sucrose and about 15% to about 35% (weight/weight) dextran. In some embodiments, the cryoprotectant solution comprises about 20% (weight/weight) sucrose and about 20% (weight/weight) dextran.

In some embodiments, the cryoprotectant solution comprises about 40% to about 80% (weight/weight) water. In some embodiments, the cryoprotectant solution comprises about 50% to about 70% (weight/weight) water. In some embodiments, the cryoprotectant solution comprises about 55% to about 65% (weight/weight) water. In some embodiments, the cryoprotectant solution comprises about 60% (weight/weight) water.

In some embodiments, the cryoprotectant solution comprises about 0.05% to about 0.6% (weight/weight) L-cysteine HCl (e.g., a form of L-cysteine). In some embodiments, the cryoprotectant solution comprises about 0.1% to about 0.5% (weight/weight) L-cysteine HCl. In some embodiments, the cryoprotectant solution comprises about 0.15% to about 0.45% (weight/weight) L-cysteine HCl. In some embodiments, the pharmaceutical agent comprises about 0.1% to about 0.25% or about 0.15% to about 0.35% (weight/weight) L-cysteine HCl.

In some embodiments, the cryoprotectant solution comprises about 0.2% (weight/weight) L-cysteine HCl.

In some embodiments, the cryoprotectant solution comprises about 59.8% (weight/weight) water.

In some embodiments, the cryoprotectant solution comprises about 0.4% (weight/weight) L-cysteine HCl.

In some embodiments, the cryoprotectant solution comprises about 59.6% (weight/weight) water.

In certain embodiments, the cryoprotectant solution provided herein comprises: (i) about 20% (weight/weight) sucrose; (ii) about 20% (weight/weight) dextran; (iii) about 0.4% (weight/weight) L-cysteine HCl; and (iv) about 59.6% (weight/weight) water.

In certain embodiments, the cryoprotectant solution provided herein comprises: (i) about 20% (weight/weight) sucrose; (ii) about 20% (weight/weight) dextran; (iii) about 0.2% (weight/weight) L-cysteine HCl; and (iv) about 59.8% (weight/weight) water.

In certain embodiments, the cryoprotectant solution provided herein comprises: (i) about 20% (weight/weight) sucrose; (ii) about 20% (weight/weight) dextran; and (iii) about 60% (weight/weight) water.

In some embodiments, the cryoprotectant (e.g., dry composition not containing water) comprises about 40% to about 60% (weight/weight) sucrose. In some embodiments, the cryoprotectant comprises about 45% to about 55% (weight/weight) sucrose. In some embodiments, the cryoprotectant comprises about 50% (weight/weight) sucrose.

In some embodiments, the cryoprotectant comprises about 40% to about 60% (weight/weight) dextran. In some embodiments, the cryoprotectant comprises about 45% to about 55% (weight/weight) dextran. In some embodiments, the cryoprotectant comprises about 50% (weight/weight) dextran.

In some embodiments, the cryoprotectant comprises about 40% to about 60% (weight/weight) sucrose and 40% to about 60% (weight/weight) dextran. In some embodiments, the cryoprotectant comprises about 45% to about 55% (weight/weight) sucrose and about 45% to about 55% (weight/weight) dextran. In some embodiments, the cryoprotectant comprises about 50% (weight/weight) sucrose and about 50% (weight/weight) dextran.

In some embodiments, the cryoprotectant comprises about 0.25% to about 5% (weight/weight) L-cysteine HCl (e.g., a form of L-cysteine). In some embodiments, the cryoprotectant comprises about 0.5% to about 2.5% (weight/weight) L-cysteine HCl. In some embodiments, the cryoprotectant comprises about 0.75% to about 1.5% (weight/weight) L-cysteine HCl. In some embodiments, the cryoprotectant comprises about 1% (weight/weight) L-cysteine HCl.

In certain embodiments, the cryoprotectant provided herein comprises: (i) about 50% (weight/weight) sucrose; (ii) about 50% (weight/weight) dextran; and (iii) about 1% (weight/weight) L-cysteine HCl.

In certain embodiments, the cryoprotectant provided herein comprises: (i) about 50% (weight/weight) sucrose; and (ii) about 50% (weight/weight) dextran.

In some embodiments, the cryoprotectant comprises a dried (dehydrated) maize glucose syrup (such as Glucidex), such as a dried maize glucose syrup with a dextrose equivalent (DE) of 21 (such as Glucidex 21). In some embodiments, the cryoprotectant comprises sucrose. In some embodiments, the cryoprotectant comprises dextrose (also referred to as glucose). In some embodiments, the cryoprotectant comprises monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and dextrose. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, and monosodium glutamate. In some embodiments, the cryoprotectant comprises dextrose and monosodium glutamate in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, monosodium glutamate, and L-cysteine HCl (e.g., a form of L-cysteine). In some embodiments, the cryoprotectant does not comprise L-cysteine HCl.

In some embodiments, the cryoprotectant (e.g., dry composition not containing water) comprises about 27% to about 47% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21).

In some embodiments, the cryoprotectant (e.g., dry composition not containing water) comprises about 27% to about 47% (weight/weight) sucrose. In some embodiments, the cryoprotectant comprises about 32% to about 42% (weight/weight) sucrose. In some embodiments, the cryoprotectant comprises about 37% (weight/weight) sucrose.

In some embodiments, the cryoprotectant comprises about 8% to about 18% (weight/weight) dextrose. In some embodiments, the cryoprotectant comprises about 11% to about 15% (weight/weight) dextrose. In some embodiments, the cryoprotectant comprises about 13% (weight/weight) dextrose.

In some embodiments, the cryoprotectant comprises about 8% to about 18% (weight/weight) monosodium glutamate. In some embodiments, the cryoprotectant comprises about 11% to about 15% (weight/weight) monosodium glutamate. In some embodiments, the cryoprotectant comprises about 13% (weight/weight) monosodium glutamate.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21) and about 27% to about 47% (weight/weight) sucrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21) and about 32% to about 42% (weight/weight) sucrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21) and about 37% (weight/weight) sucrose.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) dextrose. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) monosodium glutamate. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; about 8% to about 18% (weight/weight) dextrose; and about 8% to about 18% (weight/weight) monosodium glutamate.

In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) dextrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) monosodium glutamate. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; about 11% to about 15% (weight/weight) dextrose; and about 11% to about 15% (weight/weight) monosodium glutamate.

In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) dextrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) monosodium glutamate. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; about 13% (weight/weight) dextrose; and about 13% (weight/weight) monosodium glutamate.

In some embodiments, the cryoprotectant comprises about 0.05% to about 0.6% (weight/weight) L-cysteine HCl (e.g., a form of L-cysteine). In some embodiments, the cryoprotectant comprises about 0.1% to about 0.5% (weight/weight) L-cysteine HCl. In some embodiments, the cryoprotectant comprises about 0.1% to about 0.25% (weight/weight) L-cysteine HCl. In some embodiments, the cryoprotectant comprises about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; (iv) about 13% (weight/weight) monosodium glutamate; and (v) about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; and (iv) about 13% (weight/weight) monosodium glutamate.

In certain aspects provided herein are pharmaceutical agents that comprise bacteria. In certain embodiments, the pharmaceutical agents maintain their stability, e.g., for three, six, twelve, eighteen and/or twenty-four months under long-term (2-8° C.) and/or accelerated (23-27° C. (optionally at 60% relative humidity (RH))) storage conditions, e.g., as determined by total cell count (TCC), e.g., as determined by Coulter counter and described herein. In certain embodiments, the pharmaceutical agents maintain their stability, e.g., for three, six, twelve, eighteen and/or twenty-four months under long-term (−20° C.) and/or accelerated (2-8° C.) storage conditions, e.g., as determined by total cell count (TCC), e.g., as determined by Coulter counter and described herein.

In certain aspects provided herein are pharmaceutical agents that comprise bacteria. In some embodiments, the water content of the pharmaceutical agents is between about 0.5% and about 9/6, between about 1% and about 8%, between about 1% and about 6%, (e.g., about 1.7%, e.g., 1.8%, e.g., about 2%, e.g., about 2.2%, e.g., about 2.3%, e.g., about 2.4%, e.g., about 2.8%, e.g., about 2.9%, e.g., about 3%, e.g., about 3.1%, e.g., about 3.2%, e.g., about 3.3%, e.g., about 3.5%, e.g., about 3.6%, e.g., about 4%, e.g., about 4.5%, e.g., about 5%, e.g., about 5.3%, e.g., about 5.4%, or e.g., about 7.8%), e.g., as determined by the Karl-Fischer method for water content analysis provided in Ph. Eur. method 2.5.12, and as described herein. In some embodiments, the pharmaceutical agents maintain their water content, e.g., for three, six, twelve, eighteen and/or twenty-four months under long-term (2-8° C.) and/or accelerated (25° C. (optionally at 60% RH)) storage conditions. In some embodiments, the pharmaceutical agents maintain their water content, e.g., for three, six, twelve, eighteen and/or twenty-four months under long-term (−20° C.) and/or accelerated (2-8° C.) storage conditions.

The pharmaceutical agent can be of bacterial origin (e.g., mixture of selected strains or agents (e.g., components) thereof. The pharmaceutical agent can be of bacterial origin (e.g., a single selected strain and/or agents (e.g., components) thereof. The pharmaceutical agent can be a powder that comprises the bacteria and/or components thereof, and can comprise additional agents such as, e.g., cryoprotectant. For example, in some embodiments, the pharmaceutical agent is a freeze dried (e.g., lyophilized) powder of bacteria and/or components thereof that optionally, further comprise additional agents, such as a cryoprotectant.

In some aspects, the disclosure provides a solid dosage form (e.g., capsule, tablet, or minitablet) that comprises a pharmaceutical agent (e.g., powder) described herein. The solid dosage form can be enteric coated.

In certain embodiments, the solid dosage form comprises a capsule. In some embodiments, the capsule is a size 00, size 0, size 1, size 2, size 3, size 4, or size 5 capsule. In some embodiments, the capsule is a size 0 capsule.

In some embodiments, the solid dosage form comprises a tablet. In some embodiments, the tablet (e.g., enterically coated tablet) is a 5 mm, 6 mm, 7 mm, 8 mm, 9 mm, 10 mm, 11 mm, 12 mm, 13 mm, 14 mm, 15 mm, 16 mm, 17 mm, or 18 mm tablet.

In some embodiments, the solid dosage form comprises a minitablet. In some embodiments, the minitablet (e.g., enterically coated minitablet) is a 1 mm minitablet, 1.5 mm minitablet, 2 mm minitablet, 3 mm minitablet, or 4 mm minitablet. In some embodiments, a plurality of enterically coated minitablets are contained in a capsule (e.g., a size 0 capsule can contain about 31 to about 35 (e.g., 33) minitablets, wherein the minitablets are 3 mm in size). In some embodiments, the capsule is a size 00, size 0, size 1, size 2, size 3, size 4, or size 5 capsule. In some embodiments, the capsule comprises HPMC (hydroxyl propyl methyl cellulose) or gelatin.

In some embodiments, the enteric coating comprises one enteric coating.

In some embodiments, the enteric coating comprises an inner enteric coating and an outer enteric coating. In some embodiments, the enteric coating comprises an inner enteric coating and an outer enteric coating, and wherein the inner and outer enteric coatings are not identical (e.g., the inner and outer enteric coatings do not contain identical components in identical amounts).

In some embodiments, the enteric coating (e.g., the one enteric coating or the inner enteric coating and/or the outer enteric coating) comprises a polymethacrylate-based copolymer.

In some embodiments, the enteric coating (e.g., the one enteric coating or the inner enteric coating and/or the outer enteric coating) comprises a methacrylic acid ethyl acrylate (MAE) copolymer (1:1).

In some embodiments, the one enteric coating comprises methacrylic acid ethyl acrylate (MAE) copolymer (1:1) (such as Kollicoat MAE 100P).

In some embodiments, the one enteric coating comprises a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

In some embodiments, the enteric coating (e.g., the one enteric coating or the inner enteric coating and/or the outer enteric coating) comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

In some embodiments, the enteric coating (e.g., the one enteric coating or the inner enteric coating and/or the outer enteric coating) comprises an anionic polymeric material.

In some embodiments, the pharmaceutical agent comprises bacteria.

In some embodiments, the pharmaceutical agent has one or more beneficial immune effects outside the gastrointestinal tract, e.g., when the solid dosage form is orally administered.

In some embodiments, the pharmaceutical agent modulates immune effects outside the gastrointestinal tract in the subject, e.g., when the solid dosage form is orally administered.

In some embodiments, the pharmaceutical agent causes a systemic effect (e.g., an effect outside of the gastrointestinal tract), e.g., when the solid dosage form is orally administered.

In some embodiments, the pharmaceutical agent acts on immune cells and/or epithelial cells in the small intestine (e.g., causing a systemic effect (e.g., an effect outside of the gastrointestinal tract), e.g., when the solid dosage form is orally administered.

In some embodiments, the pharmaceutical agent comprises isolated bacteria (e.g., from one or more strains of bacteria (e.g., bacteria of interest) (e.g., a therapeutically effective amount thereof)). E.g., wherein at least 5%, at least 10%, at least 25%, at least 50%, at least 75%, at least 80%, at least 85%, at least 90%, at least 95%, or at least 99% of the content of the pharmaceutical agent is the isolated bacteria (e.g., bacteria of interest).

In some embodiments, the pharmaceutical agent comprises bacteria that have been gamma irradiated, UV irradiated, heat inactivated, acid treated, or oxygen sparged.

In some embodiments, the pharmaceutical agent comprises live bacteria.

In some embodiments, the pharmaceutical agent comprises dead bacteria.

In some embodiments, the pharmaceutical agent comprises non-replicating bacteria.

In some embodiments, the pharmaceutical agent comprises bacteria from one strain of bacteria.

In some embodiments, the bacteria are lyophilized (e.g., the lyophilized product further comprises a pharmaceutically acceptable excipient) (e.g., a powder form).

In some embodiments, the bacteria are gamma irradiated.

In some embodiments, the bacteria are UV irradiated.

In some embodiments, the bacteria are heat inactivated (e.g., at 50° C. for two hours or at 90° C. for two hours).

In some embodiments, the bacteria are acid treated.

In some embodiments, the bacteria are oxygen sparged (e.g., at 0.1 vvm for two hours).

In some embodiments, the bacteria are Gram positive bacteria.

In some embodiments, the bacteria are Gram negative bacteria.

In some embodiments, the bacteria are aerobic bacteria.

In some embodiments, the bacteria are anaerobic bacteria. In some embodiments, the anaerobic bacteria comprise obligate anaerobes. In some embodiments, the anaerobic bacteria comprise facultative anaerobes. In some embodiments, the bacteria are acidophile bacteria.

In some embodiments, the bacteria are alkaliphile bacteria.

In some embodiments, the bacteria are neutralophile bacteria.

In some embodiments, the bacteria are fastidious bacteria.

In some embodiments, the bacteria are nonfastidious bacteria.

In some embodiments, the bacteria are of a taxonomic group (e.g., class, order, family, genus, species or strain) listed in Table 1, Table 2, Table 3, or Table 4.

In some embodiments, the bacteria are a bacterial strain listed in Table 1, Table 2, Table 3, or Table 4.

In some embodiments, the bacteria are of a taxonomic group (e.g., class, order, family, genus, species or strain) listed in Table J.

In some embodiments, the bacteria are a bacterial strain listed in Table J.

In some embodiments, the Gram negative bacteria belong to class Negativicutes.

In some embodiments, the Gram negative bacteria belong to family Veillonellaceae, Selenomonadaceae, Acidaminococcaceae, or Sporomusaceae.

In some embodiments, the bacteria of the genus Megasphaera, Selenomonas, Propionospora, or Acidaminococcus.

In some embodiments, the bacteria are Megasphaera sp., Selenomonas felix, Acidaminococcus intestini, or Propionospora sp. bacteria.

In some embodiments, the bacteria are of the genus Lactococcus, Prevotella, Bifidobacterium, or Veillonella.

In some embodiments, the bacteria are Lactococcus lactis cremoris bacteria.

In some embodiments, the bacteria are Prevotella histicola bacteria.

In some embodiments, the bacteria are Bifidobacterium animalis bacteria.

In some embodiments, the bacteria are Veillonella parvula bacteria.

In some embodiments, the bacteria are Lactococcus lactis cremoris bacteria. In some embodiments, the Lactococcus lactis cremoris bacteria are a strain comprising at least 90% (or at least 97%) genomic, 16S and/or CRISPR sequence identity to the nucleotide sequence of the Lactococcus lactis cremoris Strain A (ATCC designation number PTA-125368). In some embodiments, the Lactococcus bacteria are a strain comprising at least 99/6 genomic, 16S and/or CRISPR sequence identity to the nucleotide sequence of the Lactococcus lactis cremoris Strain A (ATCC designation number PTA-125368). In some embodiments, the Lactococcus bacteria are Lactococcus lactis cremoris Strain A (ATCC designation number PTA-125368).

In some embodiments, the bacteria are Prevotella bacteria. In some embodiments, the Prevotella bacteria are a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are a strain comprising at least 99% genomic, 16S and/or CRISPR sequence identity to the nucleotide sequence of the Prevotella Strain B 50329 (NRRL accession number B 50329). In some embodiments, the Prevotella bacteria are Prevotella Strain B 50329 (NRRL accession number B 50329).

In some embodiments, the bacteria are Bifidobacterium bacteria. In some embodiments, the Bifidobacterium bacteria are from a strain comprising at least 90% (or at least 97%) genomic, 16S and/or CRISPR sequence identity to the nucleotide sequence of the Bifidobacterium bacteria deposited as ATCC designation number PTA-125097. In some embodiments, the Bifidobacterium bacteria are a strain comprising at least 99% genomic, 16S and/or CRISPR sequence identity to the nucleotide sequence of the Bifidobacterium bacteria deposited as ATCC designation number PTA-125097. In some embodiments, the Bifidobacterium bacteria are Bifidobacterium bacteria deposited as ATCC designation number PTA-125097.

In some embodiments, the bacteria are Veillonella bacteria. In some embodiments, the Veillonella bacteria are a strain comprising at least 90% (or at least 97%) genomic, 16S and/or CRISPR sequence identity to the nucleotide sequence of the Veillonella bacteria deposited as ATCC designation number PTA-125691. In some embodiments, the Veillonella bacteria are a strain comprising at least 99% genomic, 16S and/or CRISPR sequence identity to the nucleotide sequence of the Veillonella bacteria deposited as ATCC designation number PTA-125691. In some embodiments, the Veillonella bacteria are Veillonella bacteria deposited as ATCC designation number PTA-125691.

In some embodiments, the bacteria are from Ruminococcus gnavus bacteria. In some embodiments, the Ruminococcus gnavus bacteria are a strain comprising at least 90/a (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99/a genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria are Megasphaera sp. bacteria. In some embodiments, the Megasphaera sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria are Fournierella massiliensis bacteria. In some embodiments, the Fournierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria are Harryflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are a strain comprising at least 99/a genomic, 16S and/or CRISPR sequence identity to the nucleotide sequence of the Harryflintia acetispora bacteria deposited as ATCC designation number PTA-126694. In some embodiments, the Harryflintia acetispora bacteria are Harryflintia acetispora bacteria deposited as ATCC designation number PTA-126694.

In some embodiments, the bacteria are of the family Acidaminococcaceae, Alcaligenaceae, Akkermansiaceae, Bacteriodaceae, Bifidobacteriaceae, Burkholderiaceae, Catabacteriaceae, Clostridiaceae, Coriobacteriaceae, Enterobacteriaceae, Enterococcaceae, Fusobacteriaceae, Lachnospiraceae, Listeraceae, Mycobacteriaceae, Neisseriaceae, Odoribacteraceae, Oscillospiraceae, Peptococcaceae, Peptostreptococcaceae, Porphyromonadaceae, Prevotellaceae, Propionibacteraceae, Rikenellaceae, Ruminococcaceae, Selenomonadaceae, Sporomusaceae, Streptococcaceae, Streptomycetaceae, Sutterellaceae, Synergistaceae, or Veillonellaceae.

In some embodiments, the bacteria are of the genus Akkermansia, Christensenella, Blautia, Enterococcus, Eubacterium, Roseburia, Bacteroides, Parabacteroides, or Erysipelatoclostridium.

In some embodiments, the bacteria are Blautia hydrogenotrophica, Blautia stercoris, Blautia wexlerae, Eubacterium faecium, Eubacterium contortum, Eubacterium rectale, Enterococcus faecalis, Enterococcus durans, Enterococcus villorum, Enterococcus gallinarum; Bifidobacterium lactis, Bifidobacterium bifidium, Bifidobacterium longum, Bifidobacterium animalis, or Bfidobacterium breve bacteria.

In some embodiments, the bacteria are BCG (bacillus Calmette-Guerin). Parabacteroides, Blautia, Veillonella, Lactobacillus salivarius, Agathobaculum, Ruminococcus gnavus, Paraclostridium benzoelyticum, Turicibacter sanguinus, Burkholderia, Klebsiella quasipneumoniae ssp similpneumoniae, Klebsiella oxytoca, Tyzzerela nexilis, or Neisseria bacteria.

In some embodiments, the bacteria are Blautia hydrogenotrophica bacteria.

In some embodiments, the bacteria are Blautia stercoris bacteria.

In some embodiments, the bacteria are Blautia wexlerae bacteria.

In some embodiments, the bacteria are Enterococcus gallinarum bacteria.

In some embodiments, the bacteria are Enterococcus faecium bacteria.

In some embodiments, the bacteria are Bfidobacterium bifidium bacteria.

In some embodiments, the bacteria are Bfidobacterium breve bacteria.

In some embodiments, the bacteria are Bfidobacterium longum bacteria.

In some embodiments, the bacteria are Roseburia hominis bacteria.

In some embodiments, the bacteria are Bacteroides thetaiotaomicron bacteria.

In some embodiments, the bacteria are Bacteroides coprocola bacteria.

In some embodiments, the bacteria are Erysipelatoclostridium ramosum bacteria.

In some embodiments, the bacteria are Megasphera massiliensis bacteria.

In some embodiments, the bacteria are Eubacterium bacteria.

In some embodiments, the bacteria are Parabacteroides distasonis bacteria.

In some embodiments, the bacteria are Lactobacillus plantarum bacteria.

In some embodiments, the bacteria are bacteria of the Negativicutes class.

In some embodiments, the bacteria are of the Veillonellaceae family.

In some embodiments, the bacteria are of the Selenomonadaceae family.

In some embodiments, the bacteria are of the Acidaminococcaceae family.

In some embodiments, the bacteria are of the Sporomusaceae family.

In some embodiments, the bacteria are of the Megasphaera genus.

In some embodiments, the bacteria are of the Selenomonas genus.

In some embodiments, the bacteria are of the Propionospora genus.

In some embodiments, the bacteria are of the Acidaminococcus genus.

In some embodiments, the bacteria are Megasphaera sp. bacteria.

In some embodiments, the bacteria are Selenomonas felix bacteria.

In some embodiments, the bacteria are Acidaminococcus intestini bacteria.

In some embodiments, the bacteria are Propionospora sp. bacteria.

In some embodiments, the bacteria are bacteria of the Clostridia class.

In some embodiments, the bacteria are of the Oscillospriraceae family.

In some embodiments, the bacteria are of the Faecalibacterium genus.

In some embodiments, the bacteria are of the Fournierella genus.

In some embodiments, the bacteria are of the Harryflintia genus.

In some embodiments, the bacteria are of the Agathobaculum genus.

In some embodiments, the bacteria are Faecalibacterium prausnitzii (e.g., Faecalibacterium prausnitzii Strain A) bacteria.

In some embodiments, the bacteria are Fournierella massiliensis (e.g., Fournierella massiliensis Strain A) bacteria.

In some embodiments, the bacteria are Harryflintia acetispora (e.g., Harryflintia acetispora Strain A) bacteria.

In some embodiments, the bacteria are Agathobaculum sp. (e.g., Agathobaculum sp. Strain A) bacteria.

In some embodiments, the bacteria are a strain of Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the bacteria are of the class Bacteroidia [phylum Bacteroidota]. In some embodiments, the bacteria are of order Bacteroidales. In some embodiments, the bacteria are of the family Porphyromonoadaceae. In some embodiments, the bacteria are of the family Prevotellaceae. In some embodiments, the bacteria are of the class Bacteroidia wherein the cell envelope structure of the bacteria is diderm. In some embodiments, the bacteria are of the class Bacteroidia that stain Gram negative. In some embodiments, the bacteria are of the class Bacteroidia wherein the bacteria is diderm and the bacteria stain Gram negative.

In some embodiments, the bacteria are of the class Clostridia [phylum Firmicutes]. In some embodiments, the bacteria are of the order Eubacteriales. In some embodiments, the bacteria are of the family Oscillispiraceae. In some embodiments, the bacteria are of the family Lachnospiraceae. In some embodiments, the bacteria are of the family Peptostreptococcaceae. In some embodiments, the bacteria are of the family Clostridiales family XII/Incertae sedis 41. In some embodiments, the bacteria are of the class Clostridia wherein the cell envelope structure of the bacteria is monoderm. In some embodiments, the bacteria are of the class Clostridia that stain Gram negative. In some embodiments, the bacteria are of the class Clostridia that stain Gram positive. In some embodiments, the bacteria are of the class Clostridia wherein the cell envelope structure of the bacteria is monoderm and the bacteria stain Gram negative. In some embodiments, the bacteria are of the class Clostridia wherein the cell envelope structure of the bacteria is monoderm and the bacteria stain Gram positive.

In some embodiments, the bacteria are of the class Negativicutes [phylum Firmicutes]. In some embodiments, the bacteria are of the order Veillonellales. In some embodiments, the bacteria are of the family Veillonelloceae. In some embodiments, the bacteria are of the order Selenomonadales. In some embodiments, the bacteria are of the family Selenomonadaceae. In some embodiments, the bacteria are of the family Sporomusaceae. In some embodiments, the bacteria are of the class Negativicutes wherein the cell envelope structure of the bacteria is diderm. In some embodiments, the bacteria are of the class Negativicutes that stain Gram negative. In some embodiments, the bacteria are of the class Negativicutes wherein the cell envelope structure of the bacteria is diderm and the bacteria stain Gram negative.

In some embodiments, the bacteria are of the class Synergistia [phylum Synergistota]. In some embodiments, the bacteria are of the order Synergistales. In some embodiments, the bacteria are of the family Synergistaceae. In some embodiments, the bacteria are of the class Synergistia wherein the cell envelope structure of the bacteria is diderm. In some embodiments, the bacteria are of the class Synergistia that stain Gram negative. In some embodiments, the bacteria are of the class Synergistia wherein the cell envelope structure of the bacteria is diderm and the bacteria stain Gram negative.

In some embodiments, the bacteria are bacteria that produce metabolites, e.g., the bacteria produce butyrate, iosine, proprionate, or tryptophan metabolites.

In some embodiments, the bacteria produce butyrate. In some embodiments, the bacteria are from the genus Blautia; Christensella; Copracoccus; Eubacterium; Lachnosperacea; Megasphaera; or Roseburia.

In some embodiments, the bacteria produce iosine. In some embodiments, the bacteria are from the genus Bfidobacterium; Lactobacillus; or Olsenella.

In some embodiments, the bacteria produce proprionate. In some embodiments, the bacteria are from the genus Akkermansia; Bacteroides; Dialister; Eubacterium; Megasphaera; Parabacteriodes; Prevotella; Ruminococcus; or Veillonella.

In some embodiments, the bacteria produce tryptophan metabolites. In some embodiments, the bacteria are from the genus Lactobacillus or Peptostreptococcus.

In some embodiments, the bacteria are bacteria that produce inhibitors of histone deacetylase 3 (HDAC3). In some embodiments, the bacteria are from the species Bariatricus massiliensis. Faecalibacterium prausnitzii. Megasphaera massiliensis or Roseburia intestinalis.

In some embodiments, the bacteria are of the genus Cutibacterium.

In some embodiments, the bacteria are Cutibacterium avidum.

In some embodiments, the bacteria are from the genus Lactobacillus.

In some embodiments, the bacteria are from the species Lactobacillus gasseri.

In some embodiments, the bacteria are from the genus Dysosmobacter.

In some embodiments, the bacteria are from the species Dysosmobacter welbionis.

In some embodiments, the bacteria of the genus Leuconostoc.

In some embodiments, the bacteria of the genus Lactobacillus.

In some embodiments, the bacteria are of the genus Akkermansia; Bacillus; Blautia; Cupriavidus; Enhydrobacter, Faecalibacterium; Lactobacillus; Lactococcus; Micrococcus; Morganella; Propionibacterium; Proteus; Rhizobium; or Streptococcus.

In some embodiments, the bacteria are Leuconostoc holzapfelii bacteria.

In some embodiments, the bacteria are Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the bacteria are Lactobacillus casei; Lactobacillus plantarum; Lactobacillus paracasei; Lactobacillus plantarum; Lactobacillus rhamnosus; or Lactobacillus sakei bacteria.

In some embodiments, the bacteria are Megasphaera sp. bacteria (e.g., from the strain with accession number NCIMB 43385, NCIMB 43386 or NCIMB 43387).

In some embodiments, the bacteria are Megasphaera massiliensis bacteria (e.g., from the strain with accession number NCIMB 42787, NCIMB 43388 or NCIMB 43389).

In some embodiments, the bacteria are Megasphaera massiliensis bacteria (e.g., from the strain with accession number DSM 26228).

In some embodiments, the bacteria are Bacillus amyloliquefaciens bacteria (e.g., from the strain with accession number NCIMB 43088, NCIMB 43087, or NCIMB 43086).

In some embodiments, the bacteria are Parabacteroides distasonis bacteria (e.g., from the strain with accession number NCIMB 42382).

In some embodiments, the bacteria are Megasphaera massiliensis bacteria (e.g., from the strain with accession number NCIMB 43388 or NCIMB 43389), or a derivative thereof. See, e.g., WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the bacteria are Megasphaera massiliensis bacteria strain deposited under accession number NCIMB 42787, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g. genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the bacteria are Megasphaera spp. bacteria from the strain with accession number NCIMB 43385, NCIMB 43386 or NCIMB 43387, or a derivative thereof. See, e.g., WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g. genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the bacteria are Parabacteroides distasonis bacteria deposited under accession number NCIMB 42382, or a derivative thereof. See, e.g., WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g. genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the bacteria are Megasphaera massiliensis bacteria deposited under accession number DSM 26228, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

In some embodiments, the bacteria are Bacillus amyloliquefaciens bacteria (e.g., from the strain with accession number NCIMB 43088, NCIMB 43087, or NCIMB 43086, or a derivative thereof. See, e.g., WO 2019/236806. In some embodiments, the Bacillus amyloliquefaciens bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Bacillus amyloliquefaciens bacteria from the strain with accession number NCIMB 43088, NCIMB 43087, or NCIMB 43086. In some embodiments, the Bacillus amyloliquefaciens bacteria is the strain with accession number NCIMB 43088, NCIMB 43087, or NCIMB 43086. In some embodiments, the Bacillus amyloliquefaciens bacteria is the strain with accession number NCIMB 43088.

In some embodiments, the pharmaceutical agent comprises bacteria and the dose of bacteria is about 1×10⁷ to about 2×10¹² (e.g., about 3×10¹⁰ or about 1.5×10¹¹ or about 1.5×10¹²) cells (e.g., wherein cell number is determined by total cell count, which is determined by Coulter counter), wherein the dose is per capsule or tablet or per total number of minitablets in a capsule. In some embodiments, the pharmaceutical agent comprises bacteria and the dose of bacteria is about 1×10¹⁰ to about 2×10¹² (e.g., about 1.6×10¹¹ or about 8×10¹¹ or about 9.6×10¹¹ about 12.8×10¹¹ or about 1.6×10¹²) cells (e.g., wherein cell number is determined by total cell count, which is determined by Coulter counter), wherein the dose is per capsule or tablet or per total number of minitablets in a capsule.

In some embodiments, the pharmaceutical agent comprises bacteria and the dose of bacteria is about 1×10⁹, about 3×10⁹, about 5×10⁹, about 1.5×10¹⁰, about 3×10¹⁰, about 5×10¹⁰, about 1.5×10¹¹, about 1.5×10¹², or about 2×10² cells, wherein the dose is per capsule or tablet or per total number of minitablets in a capsule.

In some embodiments, the pharmaceutical agent comprises a powder comprising bacteria and the dose of the pharmaceutical agent (e.g., a powder comprising bacteria) is about 10 mg to about 3500 mg, wherein the dose is per capsule or tablet or per total number of minitablets in a capsule.

In some embodiments, the pharmaceutical agent comprises a powder comprising bacteria and the dose of the pharmaceutical agent (e.g., a powder comprising bacteria) is about 30 mg to about 1300 mg (by weight of bacteria powder) (about 25, about 30, about 35, about 50, about 75, about 100, about 120, about 150, about 250, about 300, about 350, about 400, about 500, about 600, about 700, about 750, about 800, about 900, about 1000, about 1100, about 1200, about 1250, about 1300, about 2000, about 2500, about 3000, or about 3500 mg wherein the dose is per capsule or tablet or per total number of minitablets in a capsule.

In some embodiments, the pharmaceutical agent comprises bacteria and the dose of pharmaceutical agent (e.g., bacteria) is about 2×10⁶ to about 2×10¹⁶ particles (e.g., wherein particle count is determined by NTA (nanoparticle tracking analysis)), wherein the dose is per capsule or tablet or per total number of minitablets in a capsule.

In some embodiments, the pharmaceutical agent comprises bacteria and the dose of pharmaceutical agent (e.g., bacteria) is about 5 mg to about 900 mg total protein (e.g., wherein total protein is determined by Bradford assay or BCA), wherein the dose is per capsule or tablet or per total number of minitablets in a capsule.

In some embodiments, the solid dosage form further comprises one or more additional therapeutic agents.

In some aspects, the disclosure provides a method of treating a subject (e.g., human) (e.g., a subject in need of treatment), the method comprising administering to the subject a pharmaceutical agent, a pharmaceutical composition or a solid dosage form provided herein. In some aspects, the disclosure provides use of a pharmaceutical agent, a pharmaceutical composition or a solid dosage form provided herein for the preparation of a medicament for treating a subject (e.g., human) (e.g., a subject in need of treatment).

In some embodiments, the pharmaceutical agent, pharmaceutical composition or solid dosage form is orally administered (e.g., is for oral administration).

In some embodiments, the pharmaceutical agent, pharmaceutical composition or solid dosage form is administered to a subject that is in a fed or fasting state. In some embodiments, the pharmaceutical agent, pharmaceutical composition or solid dosage form is administered to a subject on an empty stomach (e.g., one hour before eating or two hours after eating). In some embodiments, the pharmaceutical agent, pharmaceutical composition or solid dosage form is administered to a subject one hour before eating. In some embodiments, the pharmaceutical agent, pharmaceutical composition or solid dosage form is administered to a subject two hours after eating.

In some embodiments, the pharmaceutical agent, pharmaceutical composition or solid dosage form is administered (e.g., is for administration) 1, 2, 3, or 4 times a day.

In some embodiments, the pharmaceutical composition or solid dosage form provides release of the pharmaceutical agent in the small intestine, e.g., in the upper small intestine, of the pharmaceutical agent contained in the solid dosage form.

In some embodiments, the pharmaceutical composition or solid dosage form delivers the pharmaceutical agent to the small intestine, wherein the pharmaceutical agent can act on immune cells and/or epithelial cells in the small intestine, e.g., in the upper small intestine, e.g., to cause effects throughout the body (e.g., systemic effect).

In some embodiments, the pharmaceutical agent provides one or more beneficial immune effects outside the gastrointestinal tract, e.g., when orally administered.

In some embodiments, the pharmaceutical agent modulates immune effects outside the gastrointestinal tract in the subject, e.g., when orally administered.

In some embodiments, the pharmaceutical agent causes a systemic effect (e.g., an effect outside of the gastrointestinal tract), e.g., when orally administered.

In some embodiments, the pharmaceutical agent acts on immune cells and/or epithelial cells in the small intestine (e.g., upper small intestine) (e.g., causing a systemic effect (e.g., an effect outside of the gastrointestinal tract), e.g., when orally administered.

In some embodiments, the pharmaceutical agent, pharmaceutical composition or solid dosage form is administered orally and has one or more beneficial immune effects outside the gastrointestinal tract (e.g., interaction between the agent and cells in the small intestine modulates a systemic immune response).

In some embodiments, the pharmaceutical agent, pharmaceutical composition or solid dosage form is administered orally and modulates immune effects outside the gastrointestinal tract (e.g., interaction between agent and cells in the small intestine (e.g., upper small intestine) modulates a systemic immune response).

In some embodiments, the pharmaceutical agent, pharmaceutical composition or solid dosage form is administered orally and activates innate antigen presenting cells (e.g., in the small intestine, e.g., upper small intestine).

In some embodiments, the subject is in need of treatment (and/or prevention) of a cancer.

In some embodiments, the subject is in need of treatment (and/or prevention) of an autoimmune disease.

In some embodiments, the subject is in need of treatment (and/or prevention) of an inflammatory disease.

In some embodiments, the subject is in need of treatment (and/or prevention) of a metabolic disease.

In some embodiments, the subject is in need of treatment (and/or prevention) of a dysbiosis.

In some embodiments, the pharmaceutical agent, pharmaceutical composition or solid dosage form is administered in combination with a therapeutic agent (e.g., additional therapeutic agent).

In some aspects, the disclosure provides a cryoprotectant solution, e.g., for use in preparing a pharmaceutical agent, e.g, that comprises bacteria, as described herein.

In some aspects, the disclosure provides a method comprising combining bacteria (e.g., a pellet comprising bacteria) with a cryoprotectant solution, thereby preparing a formulated paste. In some embodiments, the disclosure provides a formulated paste prepared by this method.

In some embodiments, the method further comprises freeze drying the formulated paste, to thereby prepare a freeze-dried product. In some embodiments, the freeze drying comprises primary drying. In some embodiments, the freeze drying comprises primary drying and secondary drying. In some embodiments, the disclosure provides a freeze-dried product prepared by this method.

In some embodiments, the method further comprises milling the freeze-dried product, to thereby prepare a freeze-dried powder (e.g., powder, e.g., pharmaceutical agent). In some embodiments, the disclosure provides a pharmaceutical agent prepared by this method.

In some embodiments, the method further comprises combining the freeze-dried powder with one or more excipients to thereby prepare a pharmaceutical composition. In some embodiments, the disclosure provides a pharmaceutical composition prepared by this method.

In some embodiments, the cryoprotectant solution is mixed with the pellet in a ratio of about 0.2 to about 0.5 gram (g) cryoprotectant solution per gram of pellet; about 0.05 to about 0.25 gram (g) cryoprotectant solution per gram of pellet; about 0.06 to about 0.1 gram (g) cryoprotectant solution per gram of pellet; or about 0.15 to about 0.2 gram (g) cryoprotectant solution per gram of pellet. In some embodiments, the cryoprotectant solution is mixed with the pellet in a ratio of about 0.4 gram (g) cryoprotectant solution per gram of pellet. In some embodiments, the cryoprotectant solution is mixed with the pellet in a ratio of about 0.18 gram (g) cryoprotectant solution per gram of pellet. In some embodiments, the cryoprotectant solution is mixed with the pellet in a ratio of about 0.1 gram (g) cryoprotectant solution per gram of pellet. In some embodiments, the cryoprotectant solution is mixed with the pellet in a ratio of about 0.08 gram (g) cryoprotectant solution per gram of pellet.

In some embodiments, the cryoprotectant solution is mixed with the pellet at a ratio of 4% to 10% (volume/volume), e.g., 5% to 8% (volume/volume). In some embodiments, the cryoprotectant solution is mixed with the pellet at a ratio of about 6.5% (volume/volume).

In some embodiments, the cryoprotectant solution comprises sucrose. In some embodiments, the cryoprotectant solution comprises dextran. In some embodiments, the cryoprotectant solution comprises sucrose and dextran. In some embodiments, the cryoprotectant solution comprises sucrose and dextran in equivalent amounts (e.g., on a percent weight by weight basis). In some embodiments, the cryoprotectant solution comprises sucrose, dextran, and L-cysteine HCl. In some embodiments, the cryoprotectant solution does not comprise L-cysteine HCl.

In some embodiments, the cryoprotectant solution comprises about 10% to about 30% (weight/weight) sucrose. In some embodiments, the cryoprotectant solution comprises about 15% to about 35% (weight/weight) sucrose. In some embodiments, the cryoprotectant solution comprises about 20% (weight/weight) sucrose.

In some embodiments, the cryoprotectant solution comprises about 10% to about 30% (weight/weight) dextran. In some embodiments, the cryoprotectant solution comprises about 15% to about 35% (weight/weight) dextran. In some embodiments, the cryoprotectant solution comprises about 20% (weight/weight) dextran.

In some embodiments, the cryoprotectant solution comprises about 10% to about 30% (weight/weight) sucrose and about 10% to about 30% (weight/weight) dextran. In some embodiments, the cryoprotectant solution comprises about 15% to about 35% (weight/weight) sucrose and about 15% to about 35% (weight/weight) dextran. In some embodiments, the cryoprotectant solution comprises about 20% (weight/weight) sucrose and about 20% (weight/weight) dextran.

In some embodiments, the cryoprotectant solution comprises about 40% to about 80% (weight/weight) water. In some embodiments, the cryoprotectant solution comprises about 50% to about 70% (weight/weight) water. In some embodiments, the cryoprotectant solution comprises about 55% to about 65% (weight/weight) water. In some embodiments, the cryoprotectant solution comprises about 60% (weight/weight) water.

In some embodiments, the cryoprotectant solution comprises about 0.05% to about 0.6% (weight/weight) L-cysteine HCl. In some embodiments, the cryoprotectant solution comprises about 0.1% to about 0.5% (weight/weight) L-cysteine HCl. In some embodiments, the cryoprotectant solution comprises about 0.15% to about 0.45% (weight/weight) L-cysteine HCl.

In some embodiments, the cryoprotectant solution comprises about 0.2% (weight/weight) L-cysteine HCl.

In some embodiments, the cryoprotectant solution comprises about 59.8% (weight/weight) water.

In some embodiments, the cryoprotectant solution comprises about 0.4% (weight/weight) L-cysteine HCl.

In some embodiments, the cryoprotectant solution comprises about 59.6% (weight/weight) water.

In certain embodiments, the cryoprotectant solution provided herein comprises: (i) about 20% (weight/weight) sucrose; (ii) about 20% (weight/weight) dextran; (iii) about 0.4% (weight/weight) L-cysteine HCl; and (iv) about 59.6% (weight/weight) water.

In certain embodiments, the cryoprotectant solution provided herein comprises: (i) about 20% (weight/weight) sucrose; (ii) about 20% (weight/weight) dextran; (iii) about 0.2% (weight/weight) L-cysteine HCl; and (iv) about 59.8% (weight/weight) water.

In certain embodiments, the cryoprotectant solution provided herein comprises: (i) about 20% (weight/weight) sucrose; (ii) about 20% (weight/weight) dextran; and (iii) about 60% (weight/weight) water.

In some embodiments, the cryoprotectant (e.g., dry composition not containing water) comprises about 40% to about 60% (weight/weight) sucrose. In some embodiments, the cryoprotectant comprises about 45% to about 55% (weight/weight) sucrose. In some embodiments, the cryoprotectant comprises about 50% (weight/weight) sucrose.

In some embodiments, the cryoprotectant comprises about 40% to about 60% (weight/weight) dextran. In some embodiments, the cryoprotectant comprises about 45% to about 55% (weight/weight) dextran. In some embodiments, the cryoprotectant comprises about 50% (weight/weight) dextran.

In some embodiments, the cryoprotectant comprises about 40% to about 60% (weight/weight) sucrose and 40% to about 60% (weight/weight) dextran. In some embodiments, the cryoprotectant comprises about 45% to about 55% (weight/weight) sucrose and about 45% to about 55% (weight/weight) dextran. In some embodiments, the cryoprotectant comprises about 50% (weight/weight) sucrose and about 50% (weight/weight) dextran.

In some embodiments, the cryoprotectant comprises about 0.25% to about 5% (weight/weight) L-cysteine HCl (e.g., a form of L-cysteine). In some embodiments, the cryoprotectant comprises about 0.5% to about 2.5% (weight/weight) L-cysteine HCl. In some embodiments, the cryoprotectant comprises about 0.75% to about 1.5% (weight/weight) L-cysteine HCl. In some embodiments, the cryoprotectant comprises about 1% (weight/weight) L-cysteine HCl.

In certain embodiments, the cryoprotectant provided herein comprises: (i) about 50% (weight/weight) sucrose; (ii) about 50% (weight/weight) dextran; and (iii) about 1% (weight/weight) L-cysteine HCl.

In certain embodiments, the cryoprotectant provided herein comprises: (i) about 50% (weight/weight) sucrose; and (ii) about 50% (weight/weight) dextran.

In some embodiments, the freeze-dried powder comprises about 6% to about 12% (weight/weight) sucrose. In some embodiments, the freeze-dried powder comprises about 8% to about 12% (weight/weight) sucrose. In some embodiments, the freeze-dried powder comprises about 11% (weight/weight) sucrose. In some embodiments, the freeze-dried powder comprises about 6% to about 12% (weight/weight) dextran. In some embodiments, the freeze-dried powder comprises about 8% to about 12% (weight/weight) dextran. In some embodiments, the freeze-dried powder comprises about 11% (weight/weight) dextran. In some embodiments, the freeze-dried powder comprises about 6% to about 12% (weight/weight) sucrose and about 6% to about 12% (weight/weight) dextran. In some embodiments, the freeze-dried powder comprises about 8% to about 12% (weight/weight) sucrose and about 8% to about 12% (weight/weight) dextran. In some embodiments, the freeze-dried powder comprises about 11% (weight/weight) sucrose and about 11% (weight/weight) dextran. In some embodiments, the freeze-dried powder comprises about 0.01% (weight/weight) L-cysteine HCl. In some embodiments, the pharmaceutical agent comprises about 0.1% to about 0.3% (weight/weight) L-cysteine HCl. In some embodiments, the pharmaceutical agent comprises about 0.15% to about 0.25% (weight/weight) L-cysteine HCl.

In some embodiments, the pharmaceutical agent comprises about 7% to about 21% (weight/weight) sucrose. In some embodiments, the pharmaceutical agent comprises about 10% to about 19% (weight/weight) sucrose. In some embodiments, the pharmaceutical agent comprises about 15% (weight/weight) sucrose. In some embodiments, the pharmaceutical agent comprises about 7% to about 21% (weight/weight) dextran. In some embodiments, the pharmaceutical agent comprises about 10% to about 19% (weight/weight) dextran. In some embodiments, the pharmaceutical agent comprises about 15% (weight/weight) dextran. In some embodiments, the pharmaceutical agent comprises about 7% to about 21% (weight/weight) sucrose and about 7% to about 21% (weight/weight) dextran. In some embodiments, the pharmaceutical agent comprises about 10% to about 19% (weight/weight) sucrose and about 10% to about 19% (weight/weight) dextran. In some embodiments, the pharmaceutical agent comprises about 15% (weight/weight) sucrose and about 15% (weight/weight) dextran. In some embodiments, the pharmaceutical agent comprises about 0.01% (weight/weight) L-cysteine HCl. In some embodiments, the pharmaceutical agent comprises about 0.1% to about 0.4% (weight/weight) L-cysteine HCl. In some embodiments, the pharmaceutical agent comprises about 0.15% to about 0.35% (weight/weight) L-cysteine HCl.

In some embodiments, the freeze-dried powder comprises about 23% to about 30% (weight/weight) dried (dehydrated) maize glucose syrup (such as Glucidex), such as a dried maize glucose syrup with a dextrose equivalent (DE) of 21 (such as Glucidex 21). In some embodiments, the freeze-dried powder comprises about 24% to about 28% (weight/weight) dried (dehydrated) maize glucose syrup (such as Glucidex), such as a dried maize glucose syrup with a dextrose equivalent (DE) of 21 (such as Glucidex 21). In some embodiments, the freeze-dried powder comprises about 26% (weight/weight) dried (dehydrated) maize glucose syrup (such as Glucidex), such as a dried maize glucose syrup with a dextrose equivalent (DE) of 21 (such as Glucidex 21). In some embodiments, the freeze-dried powder comprises about 21% to about 29% (weight/weight) sucrose. In some embodiments, the freeze-dried powder comprises about 23% to about 27% (weight/weight) sucrose. In some embodiments, the freeze-dried powder comprises about 25% (weight/weight) sucrose. In some embodiments, the freeze-dried powder comprises about 6% to about 11% (weight/weight) dextrose. In some embodiments, the freeze-dried powder comprises about 7% to about 10% (weight/weight) dextrose. In some embodiments, the freeze-dried powder comprises about 9% (weight/weight) dextrose. In some embodiments, the freeze-dried powder comprises about 4% to about 10% (weight/weight) glutamate. In some embodiments, the freeze-dried powder comprises about 5% to about 9% (weight/weight) glutamate. In some embodiments, the freeze-dried powder comprises about 7% (weight/weight) glutamate.

In some embodiments, the freeze-dried powder comprises about 23% to about 30% (weight/weight) dried (dehydrated) maize glucose syrup (such as Glucidex), such as a dried maize glucose syrup with a dextrose equivalent (DE) of 21 (such as Glucidex 21) and about 21% to about 29% (weight/weight) sucrose. In some embodiments, the freeze-dried powder comprises about 24% to about 28% (weight/weight) dried (dehydrated) maize glucose syrup (such as Glucidex), such as a dried maize glucose syrup with a dextrose equivalent (DE) of 21 (such as Glucidex 21) and about 23% to about 27% (weight/weight) sucrose. In some embodiments, the freeze-dried powder comprises about 0.05% to about 0.6% (weight/weight) L-cysteine HCl. In some embodiments, the freeze-dried powder comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 6% to about 11% (weight/weight) dextrose. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 4% to about 10% (weight/weight) glutamate. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; about 6% to about 11% (weight/weight) dextrose; and about 4% to about 10% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); about 23% to about 27% (weight/weight) sucrose; and about 7% to about 10% (weight/weight) dextrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; and about 5% to about 9% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; about 7% to about 1% (weight/weight) dextrose; and about 5% to about 9% (weight/weight) glutamate. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 26% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 21% to about 29% (weight/weight) sucrose. In some embodiments, the pharmaceutical agent comprises about 23% to about 27% (weight/weight) sucrose. In some embodiments, the pharmaceutical agent comprises about 25% (weight/weight) sucrose. In some embodiments, the pharmaceutical agent comprises about 6% to about 11% (weight/weight) dextrose. In some embodiments, the pharmaceutical agent comprises about 7% to about 10% (weight/weight) dextrose. In some embodiments, the pharmaceutical agent comprises about 9% (weight/weight) dextrose. In some embodiments, the pharmaceutical agent comprises about 4% to about 10% (weight/weight) glutamate. In some embodiments, the pharmaceutical agent comprises about 5% to about 9% (weight/weight) glutamate. In some embodiments, the pharmaceutical agent comprises about 7% (weight/weight) glutamate.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21) and about 21% to about 29% (weight/weight) sucrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21) and about 23% to about 27% (weight/weight) sucrose. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 6% to about 11% (weight/weight) dextrose. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 4% to about 10% (weight/weight) glutamate. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; about 6% to about 11% (weight/weight) dextrose; and about 4% to about 10% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); about 23% to about 27% (weight/weight) sucrose; and about 7% to about 10% (weight/weight) dextrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; and about 5% to about 9% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; about 7% to about 1% (weight/weight) dextrose; and about 5% to about 9% (weight/weight) glutamate. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the cryoprotectant comprises a dried (dehydrated) maize glucose syrup (such as Glucidex), such as a dried maize glucose syrup with a dextrose equivalent (DE) of 21 (such as Glucidex 21). In some embodiments, the cryoprotectant comprises sucrose. In some embodiments, the cryoprotectant comprises dextrose (also referred to as glucose). In some embodiments, the cryoprotectant comprises monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and dextrose. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, and monosodium glutamate. In some embodiments, the cryoprotectant comprises dextrose and monosodium glutamate in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, monosodium glutamate, and L-cysteine HCl (e.g., a form of L-cysteine). In some embodiments, the cryoprotectant does not comprise L-cysteine HCl.

In some embodiments, the cryoprotectant (e.g., dry composition not containing water) comprises about 27% to about 47% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21).

In some embodiments, the cryoprotectant (e.g., dry composition not containing water) comprises about 27% to about 47% (weight/weight) sucrose. In some embodiments, the cryoprotectant comprises about 32% to about 42% (weight/weight) sucrose. In some embodiments, the cryoprotectant comprises about 37% (weight/weight) sucrose.

In some embodiments, the cryoprotectant comprises about 8% to about 18% (weight/weight) dextrose. In some embodiments, the cryoprotectant comprises about 11% to about 15% (weight/weight) dextrose. In some embodiments, the cryoprotectant comprises about 13% (weight/weight) dextrose.

In some embodiments, the cryoprotectant comprises about 8% to about 18% (weight/weight) monosodium glutamate. In some embodiments, the cryoprotectant comprises about 11% to about 15% (weight/weight) monosodium glutamate. In some embodiments, the cryoprotectant comprises about 13% (weight/weight) monosodium glutamate.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21) and about 27% to about 47% (weight/weight) sucrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21) and about 32% to about 42% (weight/weight) sucrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21) and about 37% (weight/weight) sucrose.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) dextrose. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) monosodium glutamate. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; about 8% to about 18% (weight/weight) dextrose; and about 8% to about 18% (weight/weight) monosodium glutamate.

In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) dextrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) monosodium glutamate. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; about 11% to about 15% (weight/weight) dextrose; and about 11% to about 15% (weight/weight) monosodium glutamate.

In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) dextrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) monosodium glutamate. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; about 13% (weight/weight) dextrose; and about 13% (weight/weight) monosodium glutamate.

In some embodiments, the cryoprotectant comprises about 0.05% to about 0.6% (weight/weight) L-cysteine HCl (e.g., a form of L-cysteine). In some embodiments, the cryoprotectant comprises about 0.1% to about 0.5% (weight/weight) L-cysteine HCl. In some embodiments, the cryoprotectant comprises about 0.1% to about 0.25% (weight/weight) L-cysteine HCl. In some embodiments, the cryoprotectant comprises about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; (iv) about 13% (weight/weight) monosodium glutamate; and (v) about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; and (iv) about 13% (weight/weight) monosodium glutamate.

In certain embodiments, the freeze-dried powder comprises about 40% to about 70% (weight/weight) dried bacteria. In certain embodiments, the pharmaceutical agent comprises about 35% to about 70% (weight/weight) bacteria (e.g., freeze dried bacteria).

In certain embodiments, the freeze-dried powder comprises about 64% (weight/weight) dried bacteria.

In certain embodiments, the freeze-dried powder comprises about 15% to about 35% (weight/weight) bacteria (e.g., freeze dried bacteria). In certain embodiments, the freeze-dried powder comprises about 18% to about 30% (weight/weight) bacteria (e.g., freeze dried bacteria). In certain embodiments, the freeze-dried powder comprises about 25% (weight/weight) bacteria (e.g., freeze dried bacteria).

In certain embodiments, the freeze-dried powder comprises Prevotella bacteria.

In certain embodiments, the freeze-dried powder comprises Veillonella bacteria.

### BRIEF DESCRIPTION OF THE DRAWINGS

FIG. 1 is a graph showing Total Cells/Gram Stability Profile over time long-term (2-8° C. (abbreviation: 5° C.)) and accelerated (25° C. (abbreviation: 25° C.)) storage conditions for Prevotella histicola Strain Batch 1. The lower trace in the graph provides values for accelerated (25° C.) storage conditions. The upper trace in the graph provides values for long-term (2-8° C. (abbreviation: 5° C.)) storage conditions. Total Cell Count (TCC) was determined by Coulter counter.

FIG. 2 is a graph showing Water Content Stability Profile overtime long-term (2-8° C.) and accelerated (25° C.) storage conditions for Prevotella histicola Strain B Batch 1. Water content was determined by the Karl Fisher method.

FIG. 3 is a graph showing Total Cells/Gram Stability Profile over time long-term (2-8° C. (abbreviation: 5° C.)) and accelerated (25° C. (abbreviation: 25° C.)) storage conditions for Prevotella histicola Strain B Batch 2. Total Cell Count (TCC) was determined by Coulter counter.

FIG. 4 is a graph showing Water Content Stability Profile over time long-term (2-8° C. (abbreviation: 5° C.)) and accelerated (25° C.) storage conditions for Prevotella histicola Strain B Batch 2. The upper trace in the graph provides values for accelerated (25° C.) storage conditions. The lower trace in the graph provides values for long-term (2-8° C. (abbreviation: 5° C.)) storage conditions. Water content was determined by the Karl Fisher method.

FIG. 5 is a graph showing Total Cells/Gram Stability Profile over time long-term (2-8° C. (abbreviation: 5° C.)) and accelerated (25° C./60% RH (abbreviation: 25° C.)) storage conditions for Veillonella parvula Strain A Batch A. The trace to 6 months in the graph provides values for accelerated (25° C.) storage conditions. The trace to 12 months in the graph provides values for long-term (2-8° C. (abbreviation: 5° C.)) storage conditions. Total Cell Count (TCC) was determined by Coulter counter.

FIG. 6 is a graph showing Water Content Stability Profile over time long-term (2-8° C.) and accelerated (25° C.) storage conditions for Veillonella parvula Strain A Batch A. The trace to 6 months in the graph provides values for accelerated (25° C.) storage conditions. The trace to 12 months in the graph provides values for long-term (2-8° C. (abbreviation: 5° C.)) storage conditions. Water content was determined by the Karl Fisher method.

FIG. 7 is a graph showing Total Cells/Gram Stability Profile over time long-term (2-8° C. (abbreviation: 5° C.)) and accelerated (25° C./60% RH (abbreviation: 25° C.)) storage conditions for Veillonella parvula Strain A Batch D. The lower trace in the graph provides values for accelerated (25° C.) storage conditions. The upper trace in the graph provides values for long-term (2-8° C. (abbreviation: 5° C.)) storage conditions. Total Cell Count (TCC) was determined by Coulter counter.

FIG. 8 is a graph showing Water Content Stability Profile over time long-term (2-8° C. (abbreviation: 5° C.)) and accelerated (25° C.) storage conditions for Veillonella parvula Strain A Batch D. Water content was determined by the Karl Fisher method.

FIG. 9 is a graph showing Total Cells/Gram Stability Profile over time long-term (2-8° C. (abbreviation: 5° C.)) and accelerated (25° C. (abbreviation: 25° C.)) storage conditions for Prevotella histicola Strain Batch 1. The trace to 6 months in the graph provides values for accelerated (25° C.) storage conditions. The trace to 18 months in the graph provides values for long-term (2-8° C. (abbreviation: 5° C.)) storage conditions. Total Cell Count (TCC) was determined by Coulter counter.

FIG. 10 is a graph showing Water Content Stability Profile overtime long-term (2-8° C.) and accelerated (25° C.) storage conditions for Prevotella histicola Strain B Batch 1. Water content was determined by the Karl Fisher method.

FIG. 11 is a graph showing Total Cells/Gram Stability Profile over time long-term (2-8° C. (abbreviation: 5° C.)) and accelerated (25° C. (abbreviation: 25° C.)) storage conditions for Prevotella histicola Strain Batch 2. The trace to 6 months in the graph provides values for accelerated (25° C.) storage conditions. The trace to 18 months in the graph provides values for long-term (2-8° C. (abbreviation: 5° C.)) storage conditions. Total Cell Count (TCC) was determined by Coulter counter.

FIG. 12 is a graph showing Water Content Stability Profile overtime long-term (2-8° C.) and accelerated (25° C.) storage conditions for Prevotella histicola Strain B Batch 2. Water content was determined by the Karl Fisher method.

FIG. 13 is a graph showing Total Cells/Gram Stability Profile over time long-term (2-8° C. (abbreviation: 5° C.)) and accelerated (25° C. (abbreviation: 25° C.)) storage conditions for Prevotella histicola Strain Batch 4. Total Cell Count (TCC) was determined by Coulter counter.

FIG. 14 is a graph showing Water Content Stability Profile over time long-term (2-8° C.) and accelerated (25° C.) storage conditions for Prevotella histicola Strain B Batch 4. Water content was determined by the Karl Fisher method.

FIG. 15 is a graph showing Total Cells/Gram Stability Profile over time long-term (2-8° C. (abbreviation: 5° C.)) and accelerated (25° C. (abbreviation: 25° C.)) storage conditions for Prevotella histicola Strain Batch 5. Total Cell Count (TCC) was determined by Coulter counter.

FIG. 16 is a graph showing Water Content Stability Profile over time long-term (2-8° C.) and accelerated (25° C.) storage conditions for Prevotella histicola Strain B Batch 5. Water content was determined by the Karl Fisher method.

FIG. 17 is a graph showing Total Cells/Gram Stability Profile over time long-term (−20° C.) and accelerated ((2-8° C. (abbreviation: 5° C.)) storage conditions for Prevotella histicola Strain Batch i. The trace to 6 months in the graph provides values for accelerated (2-8° C. (abbreviation: 5° C.)) storage conditions. The trace to 24 months in the graph provides values for long-term ((−20° C.) storage conditions. Total Cell Count (TCC) was determined by Coulter counter.

FIG. 18 is a graph showing Water Content Stability Profile over time long-term (−20° C.) and accelerated (2-8° C. (abbreviation: 5° C.)) storage conditions for Prevotella histicola Strain B Batch i. Water content was determined by the Karl Fisher method.

### DETAILED DESCRIPTION

In certain aspects, provided herein are pharmaceutical agents (such as powders) comprising bacteria (e.g., freeze dried bacteria), wherein the bacteria in the pharmaceutical agent are present at a total cell count (TCC) of at least 1×10¹¹ cells/gram of the pharmaceutical agent.

In certain aspects provided herein are pharmaceutical agents (such as powders) that comprise bacteria. In certain embodiments, the pharmaceutical agents maintain their stability, e.g., for three, six, twelve, eighteen and/or twenty-four months under long-term (2-8° C.) and/or accelerated (23-27° C. (optionally at 60% relative humidity (RH))) storage conditions, e.g., as determined by total cell count (TCC), e.g., as determined by Coulter counter and described herein. In certain embodiments, the pharmaceutical agents maintain their stability, e.g., for three, six, twelve, eighteen and/or twenty-four months under long-term (−20° C.) and/or accelerated (2-8° C.) storage conditions, e.g., as determined by total cell count (TCC), e.g., as determined by Coulter counter and described herein.

In certain aspects provided herein are pharmaceutical agents (such as powders) that comprise bacteria. In some embodiments, the water content of the pharmaceutical agents is between about 0.5% and about 9/6, between about 1% and about 8%, between about 1% and about 6%, (e.g., about 1.7%, e.g., 1.8%, e.g., about 2%, e.g., about 2.2%, e.g., about 2.3%, e.g., about 2.4%, e.g., about 2.8%, e.g., about 2.9%, e.g., about 3%, e.g., about 3.1%, e.g., about 3.2%, e.g., about 3.3%, e.g., about 3.5%, e.g., about 3.6%, e.g., about 4%, e.g., about 4.5%, e.g., about 5%, e.g., about 5.3%, e.g., about 5.4%, or e.g., about 7.8%), e.g., as determined by the Karl-Fischer method for water content analysis provided in Ph. Eur. method 2.5.12, and as described herein. In some embodiments, the pharmaceutical agents maintain their water content, e.g., for three, six, twelve, eighteen and/or twenty-four months under long-term (2-8° C.) and/or accelerated (25° C. (optionally at 60% RH)) storage conditions. In certain embodiments, the pharmaceutical agents maintain their water content, e.g., for three, six, twelve, eighteen and/or twenty-four months under long-term (−20) and/or accelerated (2-8° C.) storage conditions, e.g., as determined by total cell count (TCC), e.g., as determined by Coulter counter and described herein.

In certain aspects provided herein are pharmaceutical agents (such as powders) that comprise bacteria and a cryoprotectant.

### Definitions

The term “about” when used before a numerical value indicates that the value may vary within a reasonable range, such as within ±10%, ±5% or ±1% of the stated value.

“Adjuvant” or “Adjuvant therapy” broadly refers to an agent that affects an immunological or physiological response in a subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition such as a solid dosage form of a pharmaceutical agent as described herein) to a subject. Examples of routes of administration include oral administration, rectal administration, topical administration, inhalation (nasal) or injection. Administration by injection includes intravenous (IV), intramuscular (IM), intratumoral (IT) and subcutaneous (SC) administration. A pharmaceutical composition described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, intratumorally, topically, intravesically, by injection into or adjacent to a draining lymph node, intravenously, by inhalation or aerosol, or subcutaneously. In another preferred embodiment, a pharmaceutical composition described herein is administered orally, intratumorally, or intravenously. In another embodiment, a pharmaceutical composition described herein is administered orally.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s)” and “neoplasm(s)” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells. Environments of particular interest are the microenvironments where cancer cells reside or locate. In some instances, the microenvironment is a tumor microenvironment or a tumor draining lymph node. In other instances, the microenvironment is a pre-cancerous tissue site or the site of local administration of a composition or a site where the composition will accumulate after remote administration.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” of bacteria from two or more strains includes the physical co-existence of the bacteria, either in the same material or product or in physically connected products, as well as the temporal co-administration or co-localization of the bacteria from the two or more strains.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Dysbiosis” refers to a state of the microbiota or microbiome of the gut or other body area, including, e.g., mucosal or skin surfaces (or any other microbiome niche) in which the normal diversity and/or function of the host gut microbiome ecological networks “microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (e.g., increase or decrease) in the prevalence of one or more bacteria types (e.g., anaerobic), species and/or strains, change (e.g., increase or decrease) in diversity of the host microbiome population composition; a change (e.g., increase or reduction) of one or more populations of symbiont organisms resulting in a reduction or loss of one or more beneficial effects; overgrowth of one or more populations of pathogens (e.g., pathogenic bacteria); and/or the presence of, and/or overgrowth of, symbiotic organisms that cause disease only when certain conditions are present.

The term “ecological consortium” is a group of bacteria which trades metabolites and positively co-regulates one another, in contrast to two bacteria which induce host synergy through activating complementary host pathways for improved efficacy.

The term “effective dose” or “effective amount” is an amount of a pharmaceutical agent that is effective to achieve a desired therapeutic response in a subject for a particular agent, composition, and mode of administration.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 200%, 30%, 400%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR1O and TLRI 1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe (such as a bacterium) or other entity or substance that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “LPS mutant or lipopolysaccharide mutant” broadly refers to selected bacteria that comprises loss of LPS. Loss of LPS might be due to mutations or disruption to genes involved in lipid A biosynthesis, such as lpxA, lpxC, and lpxD. Bacteria comprising LPS mutants can be resistant to aminoglycosides and polymyxins (polymyxin B and colistin).

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila, Bacteroides caccae, Bacteroides fragilis, Bacteroides putredinis, Bacteroides thetaiotaomicron, Bacteroides vultagus, Bifidobacterium adolescentis, Bifidobacterium bifidum, Bilophila wadsworthia, Blautia, Butyrivibrio, Campylobacter gracilis, Clostridia cluster III, Clostridia cluster IV, Clostridia cluster IX (Acidaminococcaceae group), Clostridia cluster XI, Clostridia cluster XIII (Peptostreptococcus group), Clostridia cluster XIV, Clostridia cluster XV, Collinsella aerofaciens, Coprococcus, Corynebacterium sunsvallense, Desulfomonas pigra, Dorea formicigenerans, Dorea longicatena, Escherichia col, Eubacterium hadrum, Eubacterium rectale, Faecalibacteria prausnitzii, Gemella, Lactococcus, Lanchnospira, Mollicutes cluster XVI, Mollicutes cluster XVIII, Prevotella, Rothia mucilaginosa, Ruminococcus callidus, Ruminococcus gnavus, Ruminococcus torques, and Streptococcus.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more cancer-associated bacterial strains are present in a sample. In some embodiments, the microbiome profile indicates the relative or absolute amount of each bacterial strain detected in the sample. In some embodiments, the microbiome profile is a cancer-associated microbiome profile. A cancer-associated microbiome profile is a microbiome profile that occurs with greater frequency in a subject who has cancer than in the general population. In some embodiments, the cancer-associated microbiome profile comprises a greater number of or amount of cancer-associated bacteria than is normally present in a microbiome of an otherwise equivalent tissue or sample taken from an individual who does not have cancer.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

An “oncobiome” as used herein comprises tumorigenic and/or cancer-associated microbiota, wherein the microbiota comprises one or more of a virus, a bacterium, a fungus, a protist, a parasite, or another microbe.

“Oncotrophic” or “oncophilic” microbes and bacteria are microbes that are highly associated or present in a cancer microenvironment. They may be preferentially selected for within the environment, preferentially grow in a cancer microenvironment or hone to a said environment.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with no more than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

As used herein, the term “pharmaceutical agent” refers to an agent for therapeutic use. In some embodiments, a pharmaceutical agent is a composition comprising bacteria that can be used to treat and/or prevent a disease and/or condition. In some embodiments, a medicinal product, medical food, a food product, or a dietary supplement comprises a pharmaceutical agent. In some embodiments, the pharmaceutical agent is a powder that contains the bacteria. The powder can include one or more additional components in addition to the bacteria, such as a cryoprotectant.

The terms “polynucleotide,” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, the term “preventing” a disease or condition in a subject refers to administering to the subject to a pharmaceutical treatment, e.g., the administration of one or more agents (e.g., pharmaceutical agent), such that onset of at least one symptom of the disease or condition is delayed or prevented.

As used herein, a substance is “pure” if it is substantially free of other components. The terms “purify,” “purifying” and “purified” refer to a microbe preparation or other material that has been separated from at least some of the components with which it was associated either when initially produced or generated (e.g., whether in nature or in an experimental setting), or during any time after its initial production. A microbe preparation or compositions may be considered purified if it is isolated at or after production, such as from one or more other bacterial components, and a purified microbe or microbial population may contain other materials up to about 10%, about 20%, about 30%, about 40%, about 50% a, about 60%, about 70%, about 80%, about 90%, or above about 90% and still be considered “purified.” In some embodiments, purified microbes are more than about 80%, about 85%, about 90%, about 91%, about 92%, about 93%, about 94%, about 95%, about 96%, about 97%, about 98%, about 99%, or more than about 99% pure. Microbe compositions (or preparations) are, e.g., purified from residual habitat products.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold no more than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer, Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In other embodiments, the subject has another cancer. In some embodiments, the subject has undergone a cancer therapy.

As used herein, a “systemic effect” in a subject treated with a pharmaceutical composition containing bacteria (e.g., a pharmaceutical agent comprising bacteria) of the instant invention means a physiological effect occurring at one or more sites outside the gastrointestinal tract. Systemic effect(s) can result from immune modulation (e.g., via an increase and/or a reduction of one or more immune cell types or subtypes (e.g., CD8+ T cells) and/or one or more cytokines). Such systemic effect(s) may be the result of the modulation by bacteria of the instant invention on immune or other cells (such as epithelial cells) in the gastrointestinal tract which then, directly or indirectly, result in the alteration of activity (activation and/or deactivation) of one or more biochemical pathways outside the gastrointestinal tract. The systemic effect may include treating or preventing a disease or condition in a subject.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

As used herein, a value is “greater than” another value if it is higher by any amount (e.g., each of 100, 50, 20, 12, 11, 10.6, 10.1, 10.01, and 10.001 is at least 10). Similarly, as used herein, a value is “less than” another value if it is lower by any amount (e.g., each of 1, 2, 4, 6, 8, 9, 9.2, 9.4, 9.6, 9.8, 9.9, 9.99, 9.999 is no more than 10). In contrast, as used herein, a test value “is” an anchor value when the test value rounds to the anchor value (e.g., if “an ingredient mass is 10% of a total mass,” in which case 10% is the anchor value, the test values of 9.5, 9.6, 9.7, 9.8, 9.9, 10, 10.1, 10.2, 10.3, and 10.4 would also meet the “ingredient mass is 10% of the total mass” feature).

### Bacteria

The pharmaceutical agent (e.g., and pharmaceutical compositions comprising the same) disclosed herein can comprise bacteria. For example, the pharmaceutical agent disclosed herein can comprise a powder comprising bacteria. Within a pharmaceutical agent that contains bacteria, the pharmaceutical agent can contain bacteria from one or more strains.

In some embodiments, the bacteria of the pharmaceutical agent are obtained are modified to reduce toxicity or other adverse effects, to enhance delivery) (e.g., oral delivery) (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the bacteria (e.g., either alone or in combination with another therapeutic agent), and/or to enhance immune activation or suppression by the bacteria (e.g., through modified production of polysaccharides, pili, fimbriae, adhesins). In some embodiments, the engineered bacteria described herein are modified to improve bacteria manufacturing (e.g., higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may result in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

Examples of taxonomic groups (e.g., class, order, family, genus, species or strain) of bacteria that can be used as a source of bacteria for a pharmaceutical agent described herein are provided herein (e.g., listed in Table 1, Table 2, Table 3, and/or Table 4 and/or elsewhere in the specification (e.g., Table J)). In some embodiments, the bacterial strain is a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein. In some embodiments, the bacteria of the pharmaceutical agent are oncotrophic bacteria. In some embodiments, the bacteria of the pharmaceutical agent are immunomodulatory bacteria. In some embodiments, the bacteria of the pharmaceutical agent are immunostimulatory bacteria. In some embodiments, the bacteria of the pharmaceutical agent are immunosuppressive bacteria. In some embodiments, the bacteria of the pharmaceutical agent are immunomodulatory bacteria. In certain embodiments, the bacteria of the pharmaceutical agent are generated from a combination of bacterial strains provided herein. In some embodiments, the combination is a combination of at least 2, 3, 4, 5, 6, 7, 8, 9, 10, 11, 12, 13, 14, 15, 20, 25, 30, 35, 40, 45 or 50 bacterial strains. In some embodiments, the combination includes the bacteria of the pharmaceutical agent are from bacterial strains listed herein and/or bacterial strains having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein (e.g., listed in Table 1, Table 2, Table 3, and/or Table 4 and/or elsewhere in the specification (e.g., Table J)). In certain embodiments, the bacteria of the pharmaceutical agent are generated from a bacterial strain provided herein. In some embodiments, the bacteria of the pharmaceutical agent are from a bacterial strain listed herein (e.g., listed in Table 1, Table 2, Table 3, and/or Table 4 and/or elsewhere in the specification (e.g., Table J)) and/or a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein (e.g., listed in Table 1, Table 2, Table 3, and/or Table 4 and/or elsewhere in the specification (e.g., Table J)).

In some embodiments, the bacteria of the pharmaceutical agent are Gram negative bacteria.

In some embodiments, the Gram negative bacteria belong to the class Negativicutes. The Negativicutes represent a unique class of microorganisms as they are the only diderm members of the Firmicutes phylum. These anaerobic organisms can be found in the environment and are normal commensals of the oral cavity and GI tract of humans. The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestine, and Propionospora sp.

In some embodiments, the bacteria of the pharmaceutical agent are Gram positive bacteria.

In some embodiments, the bacteria of the pharmaceutical agent are aerobic bacteria.

In some embodiments, the bacteria of the pharmaceutical agent are anaerobic bacteria. In some embodiments, the anaerobic bacteria comprise obligate anaerobes. In some embodiments, the anaerobic bacteria comprise facultative anaerobes.

In some embodiments, the bacteria of the pharmaceutical agent are acidophile bacteria.

In some embodiments, the bacteria of the pharmaceutical agent are alkaliphile bacteria.

In some embodiments, the bacteria of the pharmaceutical agent are neutralophile bacteria.

In some embodiments, the bacteria of the pharmaceutical agent are fastidious bacteria.

In some embodiments, the bacteria of the pharmaceutical agent are nonfastidious bacteria.

In some embodiments, the bacteria of the pharmaceutical agent are lyophilized.

In some embodiments, the bacteria of the pharmaceutical agent are gamma irradiated (e.g., at 17.5 or 25 kGy).

In some embodiments, the bacteria of the pharmaceutical agent are UV irradiated.

In some embodiments, the bacteria of the pharmaceutical agent are heat inactivated (e.g., at 50° C. for two hours or at 90° C. for two hours).

In some embodiments, the bacteria of the pharmaceutical agent are acid treated.

In some embodiments, the bacteria of the pharmaceutical agent are oxygen sparged (e.g., at 0.1 vvm for two hours).

The phase of growth can affect the amount or properties of bacteria. For example, bacteria can be isolated at the start of the log phase of growth, midway through the log phase, and/or once stationary phase growth has been reached for a bacterial culture.

In certain embodiments, the bacteria of the pharmaceutical agent are obligate anaerobic bacteria. Examples of obligate anaerobic bacteria include gram-negative rods (including the genera of Bacteroides, Prevotella, Porphyromonas, Fusobacterium, Bilophila and Sutterella spp.), gram-positive cocci (primarily Peptostreptococcus spp.), gram-positive spore-forming (Clostridium spp.), non-spore-forming bacilli (Actinomyces, Propionibacterium, Eubacterium, Lactobacillus and Bifidobacterium spp.), and gram-negative cocci (mainly Veillonella spp.). In some embodiments, the obligate anaerobic bacteria are of a genus selected from the group consisting of Agathobaculum, Atopobium, Blautia, Burkholderia, Dielma, Longicatena, Paraclostridium, Turicibacter, and Tyzzerella.

The Negativicutes class includes the families Veillonellaceae, Selenomonadaceae, Acidaminococcaceae, and Sporomusaceae. The Negativicutes class includes the genera Megasphaera, Selenomonas. Propionospora, and Acidaminococcus. Exemplary Negativicutes species include, but are not limited to, Megasphaera sp., Selenomonas felix, Acidaminococcus intestini, and Propionospora sp.

In some embodiments, the bacteria of the pharmaceutical agent are of the Negativicutes class.

In some embodiments, the bacteria of the pharmaceutical agent are of the Veillonellaceae family.

In some embodiments, the bacteria of the pharmaceutical agent are of the Selenomonadaceae family.

In some embodiments, the bacteria of the pharmaceutical agent are of the Acidaminococcaceae family.

In some embodiments, the bacteria of the pharmaceutical agent are of the Sporomusaceae family.

In some embodiments, the bacteria of the pharmaceutical agent are of the Megasphaera genus.

In some embodiments, the bacteria of the pharmaceutical agent are of the Selenomonas genus.

In some embodiments, the bacteria of the pharmaceutical agent are of the Propionospora genus.

In some embodiments, the bacteria of the pharmaceutical agent are of the Acidaminococcus genus.

In some embodiments, the bacteria of the pharmaceutical agent are Megasphaera sp. bacteria.

In some embodiments, the bacteria of the pharmaceutical agent are Selenomonas felix bacteria.

In some embodiments, the bacteria of the pharmaceutical agent are Acidaminococcus intestini bacteria.

In some embodiments, the bacteria of the pharmaceutical agent are Propionospora sp. bacteria.

The Oscillospriraceae family within the Clostridia class of microorganisms are common commensal organisms of vertebrates.

In some embodiments, the bacteria of the pharmaceutical agent are of the Clostridia class.

In some embodiments, the bacteria of the pharmaceutical agent are of the Oscillospriraceae family.

In some embodiments, the bacteria of the pharmaceutical agent are of the Faecalibacterium genus.

In some embodiments, the bacteria of the pharmaceutical agent are of the Fournierella genus.

In some embodiments, the bacteria of the pharmaceutical agent are of the Harryflintia genus.

In some embodiments, the bacteria of the pharmaceutical agent are of the Agathobaculum genus.

In some embodiments, the bacteria of the pharmaceutical agent are Faecalibacterium prausnitzii (e.g., Faecalibacterium prausnitzii Strain A) bacteria.

In some embodiments, the bacteria of the pharmaceutical agent are Fournierella massiliensis (e.g., Fournierella massiliensis Strain A) bacteria.

In some embodiments, the bacteria of the pharmaceutical agent are Harryflintia acetispora (e.g., Harryflintia acetispora Strain A) bacteria.

In some embodiments, the bacteria of the pharmaceutical agent are Agathobaculum sp. (e.g., Agathobaculum sp. Strain A) bacteria.

In some embodiments, the bacteria of the pharmaceutical agent are bacteria of a genus selected from the group consisting of Escherichia, Klebsiella, Lactobacillus, Shigella, and Staphylococcus.

In some embodiments, the bacteria of the pharmaceutical agent are a species selected from the group consisting of Blautia massiliensis, Paraclostridium benzoelyticum, Dielma fastidiosa, Longicatena caecimuris, Lactococcus lactis cremoris, Tyzzerella nexilis, Hungatella effluvia, Klebsiella quasipneumoniae subsp. Similipneumoniae, Klebsiella oxytoca, and Veillonella tobetsuensis.

In some embodiments, the bacteria of the pharmaceutical agent are a Prevotella bacteria selected from the group consisting of Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotellafusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica Prevotella scopos, Prevotella shahii, Prevotella zoogleoformans, and Prevotella veroralis.

In some embodiments, the bacteria of the pharmaceutical agent are a strain of bacteria comprising a genomic sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number as provided in Table 3. In some embodiments, the bacteria of the pharmaceutical agent are a strain of bacteria comprising a 16S sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the 16S sequence of the strain of bacteria deposited with the ATCC Deposit number as provided in Table 3.

The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp., Selenomonas felix, Acidaminococcus intestini, and Propionospora sp.

In some embodiments, the bacteria of the pharmaceutical agent are of the Negativicutes class.

In some embodiments, the bacteria of the pharmaceutical agent are of the Veillonellaceae family.

In some embodiments, the bacteria of the pharmaceutical agent are of the Selenomonadaceae family.

In some embodiments, the bacteria of the pharmaceutical agent are of the Acidaminococcaceae family.

In some embodiments, the bacteria of the pharmaceutical agent are of the Sporomusaceae family.

In some embodiments, the bacteria of the pharmaceutical agent are of the Megasphaera genus.

In some embodiments, the bacteria of the pharmaceutical agent are of the Selenomonas genus.

In some embodiments, the bacteria of the pharmaceutical agent are of the Propionospora genus.

In some embodiments, the bacteria of the pharmaceutical agent are of the Acidaminococcus genus.

In some embodiments, the bacteria of the pharmaceutical agent are Megasphaera sp. bacteria.

In some embodiments, the bacteria of the pharmaceutical agent are Selenomonas felix bacteria.

In some embodiments, the bacteria of the pharmaceutical agent are Acidaminococcus intestini bacteria.

In some embodiments, the bacteria of the pharmaceutical agent are Propionospora sp. bacteria.

The Oscillospriraceae family within the Clostridia class of microorganisms are common commensal organisms of vertebrates.

In some embodiments, the bacteria of the pharmaceutical agent are of the Clostridia class.

In some embodiments, the bacteria of the pharmaceutical agent are of the Oscillospriraceae family.

In some embodiments, the bacteria of the pharmaceutical agent are of the Faecalibacterium genus.

In some embodiments, the bacteria of the pharmaceutical agent are of the Fournierella genus.

In some embodiments, the bacteria of the pharmaceutical agent are of the Harryflintia genus.

In some embodiments, the bacteria of the pharmaceutical agent are of the Agathobaculum genus.

In some embodiments, the bacteria of the pharmaceutical agent are Faecalibacterium prausnitzii (e.g., Faecalibacterium prausnitzii Strain A) bacteria.

In some embodiments, the bacteria of the pharmaceutical agent are Fournierella massiliensis (e.g., Fournierella massiliensis Strain A) bacteria.

In some embodiments, the bacteria of the pharmaceutical agent are Harryflintia acetispora (e.g., Harryflintia acetispora Strain A) bacteria.

In some embodiments, the bacteria of the pharmaceutical agent are Agathobaculum sp. (e.g., Agathobaculum sp. Strain A) bacteria.

In some embodiments, the bacteria of the pharmaceutical agent are a strain of Agathobaculum sp. In some embodiments, the Agathobaculum sp. strain is a strain comprising at least 95/6, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the bacteria of the pharmaceutical agent are of the class Bacteroidia [phylum Bacteroidota]. In some embodiments, the bacteria of the pharmaceutical agent are bacteria of order Bacteroidales. In some embodiments, the bacteria of the pharmaceutical agent are of the family Porphyromonoadaceae. In some embodiments, the bacteria of the pharmaceutical agent are of the family Prevotellaceae. In some embodiments, the bacteria of the pharmaceutical agent are bacteria of the class Bacteroidia wherein the cell envelope structure of the bacteria is diderm. In some embodiments, the bacteria of the pharmaceutical agent are bacteria of the class Bacteroidia that stain Gram negative. In some embodiments, the bacteria of the pharmaceutical agent are bacteria of the class Bacteroidia wherein the bacteria is diderm and the bacteria stain Gram negative.

In some embodiments, the bacteria of the pharmaceutical agent are bacteria of the class Clostridia [phylum Firmicutes]. In some embodiments, the bacteria of the pharmaceutical agent are of the order Eubacteriales. In some embodiments, the bacteria of the pharmaceutical agent are of the family Oscillispiraceae. In some embodiments, the bacteria of the pharmaceutical agent are of the family Lachnospiraceae. In some embodiments, the bacteria of the pharmaceutical agent are of the family Peptostreptococcaceae. In some embodiments, the bacteria of the pharmaceutical agent are of the family Clostridiales family XIII/Incertae sedis 41. In some embodiments, the bacteria of the pharmaceutical agent are of the class Clostridia wherein the cell envelope structure of the bacteria is monoderm. In some embodiments, the bacteria of the pharmaceutical agent are of the class Clostridia that stain Gram negative. In some embodiments, the bacteria of the pharmaceutical agent are of the class Clostridia that stain Gram positive. In some embodiments, the bacteria of the pharmaceutical agent are of the class Clostridia wherein the cell envelope structure of the bacteria is monoderm and the bacteria stain Gram negative. In some embodiments, the bacteria of the pharmaceutical agent are of the class Clostridia wherein the cell envelope structure of the bacteria is monoderm and the bacteria stain Gram positive.

In some embodiments, the bacteria of the pharmaceutical agent are of the class Negativicutes [phylum Firmicutes]. In some embodiments, the bacteria of the pharmaceutical agent are of the order Veillonellales. In some embodiments, the bacteria of the pharmaceutical agent are of the family Veillonelloceae. In some embodiments, the bacteria of the pharmaceutical agent are of the order Selenomonadales. In some embodiments, the bacteria of the pharmaceutical agent are bacteria of the family Selenomonadaceae. In some embodiments, the bacteria of the pharmaceutical agent are of the family Sporomusaceae. In some embodiments, t the bacteria of the pharmaceutical agent are of the class Negativicutes wherein the cell envelope structure of the bacteria is diderm. In some embodiments, the bacteria of the pharmaceutical agent are from bacteria of the class Negativicutes wherein the cell envelope structure of the bacteria is diderm and the bacteria stain Gram negative.

In some embodiments, the bacteria of the pharmaceutical agent are of the class Synergistia [phylum Synergistota]. In some embodiments, the bacteria of the pharmaceutical agent are of the order Synergistales. In some embodiments, the bacteria of the pharmaceutical agent are of the family Synergistaceae. In some embodiments, the bacteria of the pharmaceutical agent are of the class Synergistia wherein the cell envelope structure of the bacteria is diderm. In some embodiments, the bacteria of the pharmaceutical agent are of the class Synergistia that stain Gram negative. In some embodiments, the bacteria of the pharmaceutical agent are of the class Synergistia wherein the cell envelope structure of the bacteria is diderm and the bacteria stain Gram negative.

In some embodiments, the bacteria of the pharmaceutical agent from one strain of bacteria, e.g., a strain provided herein.

In some embodiments, the bacteria of the pharmaceutical agent are from one strain of bacteria (e.g., a strain provided herein) or from more than one strain provided herein.

In some embodiments, the bacteria of the pharmaceutical agent are Lactococcus lactis cremoris bacteria, e.g., a strain comprising at least 90% or at least 99% genomic, 16S and/or CRISPR sequence identity to the nucleotide sequence of the Lactococcus lactis cremoris Strain A (ATCC designation number PTA-125368). In some embodiments, the bacteria of the pharmaceutical agent are Lactococcus bacteria, e.g., Lactococcus lactis cremoris Strain A (ATCC designation number PTA-125368).

In some embodiments, the bacteria of the pharmaceutical agent are Prevotella bacteria, e.g., a strain comprising at least 90% or at least 99/a genomic, 16S and/or CRISPR sequence identity to the nucleotide sequence of the Prevotella Strain B 50329 (NRRL accession number B 50329). In some embodiments, the bacteria of the pharmaceutical agent are Prevotella bacteria, e.g., Prevotella Strain B 50329 (NRRL accession number B 50329).

In some embodiments, the bacteria of the pharmaceutical agent are Bifidobacterium bacteria, e.g., a strain comprising at least 90% or at least 99% genomic, 16S and/or CRISPR sequence identity to the nucleotide sequence of the Bifidobacterium bacteria deposited as ATCC designation number PTA-125097. In some embodiments, the bacteria of the pharmaceutical agent are Bifidobacterium bacteria, e.g., Bifidobacterium bacteria deposited as ATCC designation number PTA-125097.

In some embodiments, the bacteria of the pharmaceutical agent are Veillonella bacteria, e.g., a strain comprising at least 90% or at least 99% genomic, 16S and/or CRISPR sequence identity to the nucleotide sequence of the Veillonella bacteria deposited as ATCC designation number PTA-125691. In some embodiments, the bacteria of the pharmaceutical agent are Veillonella bacteria, e.g., Veillonella bacteria deposited as ATCC designation number PTA-125691.

In some embodiments, the bacteria of the pharmaceutical agent are Ruminococcus gnavus bacteria. In some embodiments, the Ruminococcus gnavus bacteria are a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria of the pharmaceutical agent are Megasphaera sp. bacteria. In some embodiments, the Megasphaera sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99/a genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria of the pharmaceutical agent are Fournierella massiliensis bacteria. In some embodiments, the Fournierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria of the pharmaceutical agent are Harryflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are Harryflintia acetispora bacteria deposited as ATCC designation number PTA-126694.

In some embodiments, the bacteria of the pharmaceutical agent are bacteria that produce metabolites, e.g., the bacteria produce butyrate, iosine, proprionate, or tryptophan metabolites.

In some embodiments, the bacteria produce butyrate. In some embodiments, the bacteria are from the genus Blautia; Christensella; Copracoccus; Eubacterium; Lachnosperacea; Megasphaera; or Roseburia.

In some embodiments, the bacteria produce iosine. In some embodiments, the bacteria are from the genus Bfidobacterium; Lactobacillus; or Olsenella.

In some embodiments, the bacteria produce proprionate. In some embodiments, the bacteria are from the genus Akkermansia; Bacteroides; Dialister; Eubacterium; Megasphaera; Parabacteriodes; Prevotella; Ruminococcus; or Veillonella.

In some embodiments, the bacteria produce tryptophan metabolites. In some embodiments, the bacteria are from the genus Lactobacillus or Peptostreptococcus.

In some embodiments, the bacteria of the pharmaceutical agent are bacteria that produce inhibitors of histone deacetylase 3 (HDAC3). In some embodiments, the bacteria are from the species Bariatricus massiliensis. Faecalibacterium prausnitzii. Megasphaera massiliensis or Roseburia intestinalis.

In some embodiments, the bacteria are of the genus Cutibacterium.

In some embodiments, the bacteria are Cutibacterium avidum.

In some embodiments, the bacteria are from the genus Lactobacillus.

In some embodiments, the bacteria are from the species Lactobacillus gasseri.

In some embodiments, the bacteria are from the genus Dysosmobacter.

In some embodiments, the bacteria are from the species Dysosmobacter welbionis.

In some embodiments, the bacteria of the genus Leuconostoc.

In some embodiments, the bacteria of the genus Lactobacillus.

In some embodiments, the bacteria are of the genus Akkermansia; Bacillus; Blautia; Cupriavidus; Enhydrobacter; Faecalibacterium; Lactobacillus; Lactococcus; Micrococcus; Morganella; Propionibacterium; Proteus; Rhizobium; or Streptococcus.

In some embodiments, the bacteria are Leuconostoc holzapfelii bacteria.

In some embodiments, the bacteria are Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the bacteria are Lactobacillus casei; Lactobacillus plantarum; Lactobacillus paracasei; Lactobacillus plantarum; Lactobacillus rhamnosus; or Lactobacillus sakei bacteria.

In some embodiments, the bacteria are Megasphaera sp. bacteria (e.g., from the strain with accession number NCIMB 43385, NCIMB 43386 or NCIMB 43387).

In some embodiments, the bacteria are Megasphaera massiliensis bacteria (e.g., from the strain with accession number NCIMB 42787, NCIMB 43388 or NCIMB 43389).

In some embodiments, the bacteria are Megasphaera massiliensis bacteria (e.g., from the strain with accession number DSM 26228).

In some embodiments, the bacteria are Bacillus amyloliquefaciens bacteria (e.g., from the strain with accession number NCIMB 43088, NCIMB 43087, or NCIMB 43086).

In some embodiments, the bacteria are Parabacteroides distasonis bacteria (e.g., from the strain with accession number NCIMB 42382).

In some embodiments, the bacteria are Megasphaera massiliensis bacteria (e.g., from the strain with accession number NCIMB 43388 or NCIMB 43389), or a derivative thereof. See, e.g., WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the bacteria are Megasphaera massiliensis bacteria strain deposited under accession number NCIMB 42787, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the bacteria are Megasphaera spp. bacteria from the strain with accession number NCIMB 43385, NCIMB 43386 or NCIMB 43387, or a derivative thereof. See, e.g., WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the bacteria are Parabacteroides distasonis bacteria deposited under accession number NCIMB 42382, or a derivative thereof. See, e.g., WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the bacteria are Megasphaera massiliensis bacteria deposited under accession number DSM 26228, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

In some embodiments, the bacteria are Bacillus amyloliquefaciens bacteria (e.g., from the strain with accession number NCIMB 43088, NCIMB 43087, or NCIMB 43086, or a derivative thereof. See, e.g., WO 2019/236806. In some embodiments, the Bacillus amyloliquefaciens bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Bacillus amyloliquefaciens bacteria from the strain with accession number NCIMB 43088, NCIMB 43087, or NCIMB 43086. In some embodiments, the Bacillus amyloliquefaciens bacteria is the strain with accession number NCIMB 43088, NCIMB 43087, or NCIMB 43086. In some embodiments, the Bacillus amyloliquefaciens bacteria is the strain with accession number NCIMB 43088.

TABLE 1
Bacteria by Class

| Class | Order | Family | Genus* | Species |
|---|---|---|---|---|
| Actinobacter | Actinomycetales | Mycobacteriaceae | Mycobacterium | |
| | | Streptomycetaceae | Streptomyces (S.) | S. lividans, S |
| | | | | coelicolor, S sudanesis, |
| | | | | S somaliensis |
| | Bifidobacteriales | Bifidobacteriaceae | Bifidobacterium | B. adolescentis, |
| | | | (B.) | B. animalis, B. bifidum, |
| | | | | B. breve, B. lactis, B. |
| | | | | longum, B. |
| | | | | pseudocatenulatum |
| | Coriobacteriales | Coriobacteriaceae | Collinsella | Collinsella aerofaciens |
| | | | Olsenella | Olsenella faecalis |
| | Propionibacteriales | Propionibacteraceae | Propionibacterium | |
| | | | Cutibacterium | Cutibacterium avidum |
| Bacilli | Bacillales | Bacillales incertae | Gemella (G.) | G. haemolysans, G. |
| | | sedis family XI | | morbillorum |
| | | Listeraceae | Listeria (L.) | L. monocytogenes, L. |
| | | | | welshimeri |
| | Lactobacilluses | Enterococcaceae | Enterococcus (E.) | E. durans, E. faecium, |
| | | | | E. faecalis, E. |
| | | | | gallinarum, E. |
| | | | | villorum, |
| | | | Lactobacillus (L.) | L. casei, L. fermentum, |
| | | | | L. mucosae, L. |
| | | | | plantarum, L. reuteri, |
| | | | | L. rhamnosus, L. |
| | | | | salvarius, L. gasseri |
| | | Streptococcaceae | Lactococcus | Lactococcus lactis |
| | | | | cremoris |
| | | | Staphylococcus | Staphylococcus aureus |
| | | | Streptococcus (S.) | S. agalactiae, S. |
| | | | | aureus, S. australi, S. |
| | | | | mutans, S. |
| | | | | parasanguinis, S. |
| | | | | pneumoniae, S. |
| | | | | pyogenes, S. salivarius |
| Bacteriodes | Bacteroidales | Bacteriodaceae | Bacteriodes (B.) | B. caccae, B. |
| | | | | cellulosilyticus, B. |
| | | | | coprocola, B. dorei, B. |
| | | | | fragilis, B. ovatus, B. |
| | | | | putredinis, B. |
| | | | | salanitronis, B. |
| | | | | thetaiotaomicron, B. |
| | | | | vulgatus |
| | | Odoribacteraceae | Odoribacter | Odoribacter |
| | | | | splanchnicus |
| | | Porphyromonadaceae | Parabacteriodes | P. distasonis, P. |
| | | | (P.) | goldsteinii, P. merdae |
| | | | Porphyromonas | Porphyromonas |
| | | | | gingivalis |
| | | Prevotellaceae | Prevotella (P.) | P. albensis, P. amnii, P. |
| | | | | aurantiaca, P. |
| | | | | baroniae, P. bergensis, |
| | | | | P. bivia, P. brevis, P. |
| | | | | bryantii, P. buccae, P. |
| | | | | buccalis, P. colorans, |
| | | | | P. corporis, P. copri, P. |
| | | | | dentalis, P. dentasini, |
| | | | | P. denticola, P. |
| | | | | disiens,, P. enoeca, P. |
| | | | | falsenii, P. fusca, P. |
| | | | | heparinolytica, P. |
| | | | | histicola, P. |
| | | | | intermedia, P. jejuni,, |
| | | | | P. loescheii, P. |
| | | | | maculosa, P. marshii, |
| | | | | P. melaninogenica, P. |
| | | | | micans, P. multiformis, |
| | | | | P. multisaccharivorax, |
| | | | | P. nanceiensis, P. |
| | | | | nigrescens, P. oralis, P. |
| | | | | oris, , P. oryzae, P. |
| | | | | oulorum, P. pallens, P. |
| | | | | paludivivens, P. |
| | | | | pleuritidis P. |
| | | | | ruminicola, P. |
| | | | | saccharolytica, P. |
| | | | | salivae, P. scopos, P. |
| | | | | shahii, P. stercorea, P. |
| | | | | tannerae, P. |
| | | | | timonensis, P. |
| | | | | veroralis, P. |
| | | | | zoogleoformans |
| | | Rikenellaceae | Alstipes (A.) | A. communis, A. |
| | | | | dispar, A. finegoldii, A. |
| | | | | indistinctus, A. ihumii, |
| | | | | A. inops, A. |
| | | | | massiliensis, A. |
| | | | | megaguti, A. obesi, A. |
| | | | | onderdonkii, A. |
| | | | | provencensis, A. |
| | | | | putredinis, A. |
| | | | | senegalensis, A. |
| | | | | shahii, A. timonensis |
| Betaproteobacteria | Burkholderiales | Alcaligenaceae | Paenalcaligenes | Paenalcaligenes |
| | | | | hominis |
| | | | Bordella | Bordella pertussis |
| | | Burkholderiaceae | Burkholderia (B.) | B. mallei, B. |
| | | | | pseudomallei |
| | | | Ralstonia | Ralstonia solanacearum |
| | | Neisseriaceae | Neisseria | Neisseria meningitidis |
| | | Sutterellaceae | Sutterella (S.) | S. parvirubra, S. |
| | | | | stercoricanis, S. |
| | | | | wadsworthensis |
| Clostridia | Clostridiales | Catabacteriaceae | Catabacter | Catabacter |
| | | | | hongkongensis |
| | | Clostridiaceae | Aminiphila | Anaerosphaera |
| | | | | aminiphila |
| | | | Christensenellaceae | C. massiliensis, C. |
| | | | (C.) | minuta, C. timonensis |
| | | | Hungatella | Hungatella effluvia |
| | | Eubacteriaceae | Eubacterium (E.) | E. contortum, E. |
| | | | | eligens, E. faecium, E. |
| | | | | hadrum, E. hallii, E. |
| | | | | limosum, E. ramulus, |
| | | | | E. rectale |
| | | Lachnospiraceae | Anaerostipes (A.) | A. caccae, A. hadrus |
| | | | Blautia (B.) | B. hydrogenotrophica, |
| | | | | B. massiliensis, B. |
| | | | | stercoris, B. wexlerae |
| | | | Catonella | Catonella morbi |
| | | | Coprococcus (C.) | C. catus, C. comes, C. |
| | | | | eutactus |
| | | | Dialister (D.) | D. invisus, D. |
| | | | | micraeophilus, D. |
| | | | | succinatiphilus |
| | | | Dorea (D.) | D. formicigenerans, D. |
| | | | | longicatena |
| | | | Johnsonella | Johnsonella ignava |
| | | | Oribacterium (O.) | O. parvum, O. sinus |
| | | | Lachnobacterium | |
| | | | Lachnoclostridium | |
| | | | Lacrimispora (L.) | L. sacchaarolytica |
| | | | Roseburia (R.) | R. hominis, R. |
| | | | | intestinalis |
| | | | Tyzzerella | Tyzzerella nexilis |
| | | Oscillospiraceae | Oscillibacter | Oscillibacter |
| | | | | valericigenes |
| | | | Harryflintia | Harryflinta acetispora |
| | | | Dysosmobacter | Dysosmobacter |
| | | | | welbionis |
| | | Peptococcaceae | | |
| | | Peptostreptococcaceae | Paraclostridium | Paraclostridium |
| | | | | benzoelyticum |
| | | | Peptostreptococcus | Peptostreptococcus |
| | | | | russellii |
| | | Ruminococcaceae | Agathobaculum | Agathobaculum sp. |
| | | | Fournierella | Fournierella |
| | | | | masssiliensis |
| | | | Ruminococcus (R.) | R. albus, R. bromii, R. |
| | | | | callidus, R. gnavus, R. |
| | | | | inulinivorans, R. |
| | | | | obeum, R. torques |
| | | | Faecalibacterium | Faecalibacterium |
| | | | | prasusnitzii |
| | | Clostridiales family | | Intestimonas |
| | | XIII/ Incertae sedis | | butyriciproducens |
| Fusobacteria | Fusobacteriales | Fusobacteriaceae | Fusobacterium (F.) | F. nucleatum, F. |
| | | | | naviforme |
| | | Leptotrichiaceae | Leptotrichia | |
| | | | Sneathia | |
| Gammaproteobacteria | Enterobacterales | Enterobacteriaceae | Klebsiella (K.) | K. oxytoca, K. |
| | | | | pneumoniae, K. |
| | | | | quasipneumoniae |
| | | | | subsp. |
| | | | | Similipneumoniae, |
| | | | Escherichia (E.) | E. coli strain Nissle |
| | | | | 1917 (EcN), E. coli |
| | | | | strain ECOR12, E. coli |
| | | | | strain ECOR63 |
| | | | Shigella | |
| Negativicutes | | Acidaminococcaceae | Acidaminococcus | A. fermentans, A. |
| | | | (A.) | intestine |
| | | | Phascolarctobacterium | P. faecium, P. |
| | | | (P.) | succinatutens |
| | | Selenomonadaceae | Selenomonas (S.) | S. felix, S. incertae |
| | | | | sedis, S. sputigena |
| | | Sporomusaceae | Selenomonadales | |
| | | Veillonellaceae | Allisonella | |
| | | | Anaeroglobus | Anaeroglobus |
| | | | | germinatus |
| | | | Caecibacter | |
| | | | Colibacter | |
| | | | Megasphaera (M.) | M. elsedenii, M. |
| | | | | massiliensis, M. |
| | | | | micronuciformis, |
| | | | | Megasphaera sp |
| | | | Massilibacillus | Massilibacillus |
| | | | | massiliensis |
| | | | Propionispira | |
| | | | Negativicoccus | Negativicoccus |
| | | | | succinicivornas |
| | | | Veillonella (V.) | V. dispar, V. parvula, |
| | | | | V. ratti, V. |
| | | | | tobetsuensis |
| | Synergistales | Synergistaceae | Aminobacterium | Aminobacterium |
| | | | | mobile |
| | | | Cloacibacillus | Cloacibacillus |
| | | | | evryensis |
| | | | Rarimicrobium | Rarimicrobium |
| | | | | hominis |
| Verrucomicrobia | Verrucomicrobiales | Akkermansiaceae | Akkermansia | Akkermansia |
| | | | | mucinophila |

TABLE 2
Exemplary Bacterial Strains

| | Public DB |
|---|---|
| OTU | Accession |
| Actinobacillus actinomycetemcomitans | AY362885 |
| Actinobacillus minor | ACFT01000025 |
| Actinobacillus pleuropneumoniae | NR_074857 |
| Actinobacillus succinogenes | CP000746 |
| Actinobacillus ureae | AEVG01000167 |
| Actinobaculum massiliae | AF487679 |
| Actinobaculum schaalii | AY957507 |
| Actinobaculum sp. BM#101342 | AY282578 |
| Actinobaculum sp. P2P_19 P1 | AY207066 |
| Akkermansia muciniphila | CP001071 |
| Alistipes finegoldii | NR_043064 |
| Alistipes indistinctus | AB490804 |
| Alistipes onderdonkii | NR_043318 |
| Alistipes putredinis | ABFK02000017 |
| Alistipes shahii | FP929032 |
| Alistipes sp. HGB5 | AENZ01000082 |
| Alistipes sp. JC50 | JF824804 |
| Alistipes sp. RMA 9912 | GQ140629 |
| Anaerostipes caccae | ABAX03000023 |
| Anaerostipes sp. 3_2_56FAA | ACWB01000002 |
| Bacillus aeolius | NR_025557 |
| Bacillus aerophilus | NR_042339 |
| Bacillus aestuarii | GQ980243 |
| Bacillus alcalophilus | X76436 |
| Bacillus amyloliquefaciens | NR_075005 |
| Bacillus anthracis | AAEN01000020 |
| Bacillus atrophaeus | NR_075016 |
| Bacillus badius | NR_036893 |
| Bacillus cereus | ABDJ01000015 |
| Bacillus circulans | AB271747 |
| Bacillus clausii | FN397477 |
| Bacillus coagulans | DQ297928 |
| Bacillus firmus | NR_025842 |
| Bacillus flexus | NR_024691 |
| Bacillus fordii | NR_025786 |
| Bacillus gelatini | NR_025595 |
| Bacillus halmapalus | NR_026144 |
| Bacillus halodurans | AY144582 |
| Bacillus herbersteinensis | NR_042286 |
| Bacillus horti | NR_036860 |
| Bacillus idriensis | NR_043268 |
| Bacillus lentus | NR_040792 |
| Bacillus licheniformis | NC_006270 |
| Bacillus megaterium | GU252124 |
| Bacillus nealsonii | NR_044546 |
| Bacillus niabensis | NR_043334 |
| Bacillus niacini | NR_024695 |
| Bacillus pocheonensis | NR_041377 |
| Bacillus pumilus | NR_074977 |
| Bacillus safensis | JQ624766 |
| Bacillus simplex | NR_042136 |
| Bacillus sonorensis | NR_025130 |
| Bacillus sp. 10403023 MM10403188 | CAET01000089 |
| Bacillus sp. 2_A_57_CT2 | ACWD01000095 |
| Bacillus sp. 2008724126 | GU252108 |
| Bacillus sp. 2008724139 | GU252111 |
| Bacillus sp. 7_16AIA | FN397518 |
| Bacillus sp. 9_3AIA | FN397519 |
| Bacillus sp. AP8 | JX101689 |
| Bacillus sp. B27(2008) | EU362173 |
| Bacillus sp. BT1B_CT2 | ACWC01000034 |
| Bacillus sp. GB1.1 | FJ897765 |
| Bacillus sp. GB9 | FJ897766 |
| Bacillus sp. HU19.1 | FJ897769 |
| Bacillus sp. HU29 | FJ897771 |
| Bacillus sp. HU33.1 | FJ897772 |
| Bacillus sp. JC6 | JF824800 |
| Bacillus sp. oral taxon F26 | HM099642 |
| Bacillus sp. oral taxon F28 | HM099650 |
| Bacillus sp. oral taxon F79 | HM099654 |
| Bacillus sp. SRC_DSF1 | GU797283 |
| Bacillus sp. SRC_DSF10 | GU797292 |
| Bacillus sp. SRC_DSF2 | GU797284 |
| Bacillus sp. SRC_DSF6 | GU797288 |
| Bacillus sp. tc09 | HQ844242 |
| Bacillus sp. zh168 | FJ851424 |
| Bacillus sphaericus | DQ286318 |
| Bacillus sporothermodurans | NR_026010 |
| Bacillus subtilis | EU627588 |
| Bacillus thermoamylovorans | NR_029151 |
| Bacillus weihenstephanensis | NR_074926 |
| Bacteroidales bacterium ph8 | JN837494 |
| Bacteroidales genomosp. P1 | AY341819 |
| Bacteroidales genomosp. P2 oral clone MB1_G13 | DQ003613 |
| Bacteroidales genomosp. P3 oral clone MB1_G34 | DQ003615 |
| Bacteroidales genomosp. P4 oral clone MB2_G17 | DQ003617 |
| Bacteroidales genomosp. P5 oral clone MB2_P04 | DQ003619 |
| Bacteroidales genomosp. P6 oral clone MB3_C19 | DQ003634 |
| Bacteroidales genomosp. P7 oral clone MB3_P19 | DQ003623 |
| Bacteroidales genomosp. P8 oral clone MB4_G15 | DQ003626 |
| Bacteroides acidifaciens | NR_028607 |
| Bacteroides barnesiae | NR_041446 |
| Bacteroides caccae | EU136686 |
| Bacteroides cellulosilyticus | ACCH01000108 |
| Bacteroides clarus | AFBM01000011 |
| Bacteroides coagulans | AB547639 |
| Bacteroides coprocola | ABIY02000050 |
| Bacteroides coprophilus | ACBW01000012 |
| Bacteroides dorei | ABWZ01000093 |
| Bacteroides eggerthii | ACWG01000065 |
| Bacteroides faecis | GQ496624 |
| Bacteroides finegoldii | AB222699 |
| Bacteroides fluxus | AFBN01000029 |
| Bacteroides fragilis | AP006841 |
| Bacteroides galacturonicus | DQ497994 |
| Bacteroides helcogenes | CP002352 |
| Bacteroides heparinolyticus | JN867284 |
| Bacteroides intestinalis | ABJL02000006 |
| Bacteroides massiliensis | AB200226 |
| Bacteroides nordii | NR_043017 |
| Bacteroides oleiciplenus | AB547644 |
| Bacteroides ovatus | ACWH01000036 |
| Bacteroides pectinophilus | ABVQ01000036 |
| Bacteroides plebeius | AB200218 |
| Bacteroides pyogenes | NR_041280 |
| Bacteroides salanitronis | CP002530 |
| Bacteroides salyersiae | EU136690 |
| Bacteroides sp. 1_1_14 | ACRP01000155 |
| Bacteroides sp. 1_1_30 | ADCL01000128 |
| Bacteroides sp. 1_1_6 | ACIC01000215 |
| Bacteroides sp. 2_1_22 | ACPQ01000117 |
| Bacteroides sp. 2_1_56FAA | ACWI01000065 |
| Bacteroides sp. 2_2_4 | ABZZ01000168 |
| Bacteroides sp. 20_3 | ACRQ01000064 |
| Bacteroides sp. 3_1_19 | ADCJ01000062 |
| Bacteroides sp. 3_1_23 | ACRS01000081 |
| Bacteroides sp. 3_1_33FAA | ACPS01000085 |
| Bacteroides sp. 3_1_40A | ACRT01000136 |
| Bacteroides sp. 3_2_5 | ACIB01000079 |
| Bacteroides sp. 315_5 | FJ848547 |
| Bacteroides sp. 31SF15 | AJ583248 |
| Bacteroides sp. 31SF18 | AJ583249 |
| Bacteroides sp. 35AE31 | AJ583244 |
| Bacteroides sp. 35AE37 | AJ583245 |
| Bacteroides sp. 35BE34 | AJ583246 |
| Bacteroides sp. 35BE35 | AJ583247 |
| Bacteroides sp. 4_1_36 | ACTC01000133 |
| Bacteroides sp. 4_3_47FAA | ACDR02000029 |
| Bacteroides sp. 9_1_42FAA | ACAA01000096 |
| Bacteroides sp. AR20 | AF139524 |
| Bacteroides sp. AR29 | AF139525 |
| Bacteroides sp. B2 | EU722733 |
| Bacteroides sp. D1 | ACAB02000030 |
| Bacteroides sp. D2 | ACGA01000077 |
| Bacteroides sp. D20 | ACPT01000052 |
| Bacteroides sp. D22 | ADCK01000151 |
| Bacteroides sp. F_4 | AB470322 |
| Bacteroides sp. NB_8 | AB117565 |
| Bacteroides sp. WH2 | AY895180 |
| Bacteroides sp. XB12B | AM230648 |
| Bacteroides sp. XB44A | AM230649 |
| Bacteroides stercoris | ABFZ02000022 |
| Bacteroides thetaiotaomicron | NR_074277 |
| Bacteroides uniformis | AB050110 |
| Bacteroides ureolyticus | GQ167666 |
| Bacteroides vulgatus | CP000139 |
| Bacteroides xylanisolvens | ADKP01000087 |
| Bacteroidetes bacterium oral taxon D27 | HM099638 |
| Bacteroidetes bacterium oral taxon F31 | HM099643 |
| Bacteroidetes bacterium oral taxon F44 | HM099649 |
| Barnesiella intestinihominis | AB370251 |
| Bifidobacteriaceae genomosp. C1 | AY278612 |
| Bifidobacterium adolescentis | AAXD02000018 |
| Bifidobacterium angulatum | ABYS02000004 |
| Bifidobacterium animalis | CP001606 |
| Bifidobacterium bifidum | ABQP01000027 |
| Bifidobacterium breve | CP002743 |
| Bifidobacterium catenulatum | ABXY01000019 |
| Bifidobacterium dentium | CP001750 |
| Bifidobacterium gallicum | ABXB03000004 |
| Bifidobacterium infantis | AY151398 |
| Bifidobacterium kashiwanohense | AB491757 |
| Bifidobacterium longum | ABQQ01000041 |
| Bifidobacterium pseudocatenulatum | ABXX02000002 |
| Bifidobacterium pseudolongum | NR_043442 |
| Bifidobacterium scardovii | AJ307005 |
| Bifidobacterium sp. HM2 | AB425276 |
| Bifidobacterium sp. HMLN12 | JF519685 |
| Bifidobacterium sp. M45 | HM626176 |
| Bifidobacterium sp. MSX5B | HQ616382 |
| Bifidobacterium sp. TM_7 | AB218972 |
| Bifidobacterium thermophilum | DQ340557 |
| Bifidobacterium urinalis | AJ278695 |
| Blautia coccoides | AB571656 |
| Blautia glucerasea | AB588023 |
| Blautia glucerasei | AB439724 |
| Blautia hansenii | ABYU02000037 |
| Blautia hydrogenotrophica | ACBZ01000217 |
| Blautia luti | AB691576 |
| Blautia producta | AB600998 |
| Blautia schinkii | NR_026312 |
| Blautia sp. M25 | HM626178 |
| Blautia stercoris | HM626177 |
| Blautia wexlerae | EF036467 |
| Bordetella bronchiseptica | NR_025949 |
| Bordetella holmesii | AB683187 |
| Bordetella parapertussis | NR_025950 |
| Bordetella pertussis | BX640418 |
| Borrelia afzelii | ABCU01000001 |
| Borrelia burgdorferi | ABGI01000001 |
| Borrelia crocidurae | DQ057990 |
| Borrelia duttonii | NC_011229 |
| Borrelia garinii | ABJV01000001 |
| Borrelia hermsii | AY597657 |
| Borrelia hispanica | DQ057988 |
| Borrelia persica | HM161645 |
| Borrelia recurrentis | AF107367 |
| Borrelia sp. NE49 | AJ224142 |
| Borrelia spielmanii | ABKB01000002 |
| Borrelia turicatae | NC_008710 |
| Borrelia valaisiana | ABCY01000002 |
| Brucella ovis | NC_009504 |
| Brucella sp. 83_13 | ACBQ01000040 |
| Brucella sp. BO1 | EU053207 |
| Brucella suis | ACBK01000034 |
| Burkholderia ambifaria | AAUZ01000009 |
| Burkholderia cenocepacia | AAHI01000060 |
| Burkholderia cepacia | NR_041719 |
| Burkholderia mallei | CP000547 |
| Burkholderia multivorans | NC_010086 |
| Burkholderia oklahomensis | DQ108388 |
| Burkholderia pseudomallei | CP001408 |
| Burkholderia rhizoxinica | HQ005410 |
| Burkholderia sp. 383 | CP000151 |
| Burkholderia xenovorans | U86373 |
| Burkholderiales bacterium 1_1_47 | ADCQ01000066 |
| Butyrivibrio crossotus | ABWN01000012 |
| Butyrivibrio fibrisolvens | U41172 |
| Chlamydia muridarum | AE002160 |
| Chlamydia psittaci | NR_036864 |
| Chlamydia trachomatis | U68443 |
| Chlamydiales bacterium NS11 | JN606074 |
| Citrobacter amalonaticus | FR870441 |
| Citrobacter braakii | NR_028687 |
| Citrobacter farmeri | AF025371 |
| Citrobacter freundii | NR_028894 |
| Citrobacter gillenii | AF025367 |
| Citrobacter koseri | NC_009792 |
| Citrobacter murliniae | AF025369 |
| Citrobacter rodentium | NR_074903 |
| Citrobacter sedlakii | AF025364 |
| Citrobacter sp. 30_2 | ACDJ01000053 |
| Citrobacter sp. KMSI_3 | GQ468398 |
| Citrobacter werkmanii | AF025373 |
| Citrobacter youngae | ABWL02000011 |
| Cloacibacillus evryensis | GQ258966 |
| Clostridiaceae bacterium END_2 | EF451053 |
| Clostridiaceae bacterium JC13 | JF824807 |
| Clostridiales bacterium 1_7_47FAA | ABQR01000074 |
| Clostridiales bacterium 9400853 | HM587320 |
| Clostridiales bacterium 9403326 | HM587324 |
| Clostridiales bacterium oral clone P4PA_66 P1 | AY207065 |
| Clostridiales bacterium oral taxon 093 | GQ422712 |
| Clostridiales bacterium oral taxon F32 | HM099644 |
| Clostridiales bacterium ph2 | JN837487 |
| Clostridiales bacterium SY8519 | AB477431 |
| Clostridiales genomosp. BVAB3 | CP001850 |
| Clostridiales sp. SM4_1 | FP929060 |
| Clostridiales sp. SS3_4 | AY305316 |
| Clostridiales sp. SSC_2 | FP929061 |
| Clostridium acetobutylicum | NR_074511 |
| Clostridium aerotolerans | X76163 |
| Clostridium aldenense | NR_043680 |
| Clostridium aldrichii | NR_026099 |
| Clostridium algidicarnis | NR_041746 |
| Clostridium algidixylanolyticum | NR_028726 |
| Clostridium aminovalericum | NR_029245 |
| Clostridium amygdalinum | AY353957 |
| Clostridium argentinense | NR_029232 |
| Clostridium asparagiforme | ACCJ01000522 |
| Clostridium baratii | NR_029229 |
| Clostridium bartlettii | ABEZ02000012 |
| Clostridium beijerinckii | NR_074434 |
| Clostridium bifermentans | X73437 |
| Clostridium boltede | ABCC02000039 |
| Clostridium botulinum | NC_010723 |
| Clostridium butyricum | ABDT01000017 |
| Clostridium cadaveris | AB542932 |
| Clostridium carboxidivorans | FR733710 |
| Clostridium carnis | NR_044716 |
| Clostridium celatum | X77844 |
| Clostridium celerecrescens | JQ246092 |
| Clostridium cellulosi | NR_044624 |
| Clostridium chauvoei | EU106372 |
| Clostridium citroniae | ADLJ01000059 |
| Clostridium clariflavum | NR_041235 |
| Clostridium clostridiiformes | M59089 |
| Clostridium clostridioforme | NR_044715 |
| Clostridium coccoides | EF025906 |
| Clostridium cochlearium | NR_044717 |
| Clostridium cocleatum | NR_026495 |
| Clostridium colicanis | FJ957863 |
| Clostridium colinum | NR_026151 |
| Clostridium difficile | NC_013315 |
| Clostridium disporicum | NR_026491 |
| Clostridium estertheticum | NR_042153 |
| Clostridium fallax | NR_044714 |
| Clostridium favososporum | X76749 |
| Clostridium felsineum | AF270502 |
| Clostridium frigidicarnis | NR_024919 |
| Clostridium gasigenes | NR_024945 |
| Clostridium ghonii | AB542933 |
| Clostridium glycolicum | FJ384385 |
| Clostridium glycyrrhizinilyticum | AB233029 |
| Clostridium haemolyticum | NR_024749 |
| Clostridium hathewayi | AY552788 |
| Clostridium hiranonis | AB023970 |
| Clostridium histolyticum | HF558362 |
| Clostridium hylemonde | AB023973 |
| Clostridium indolis | AF028351 |
| Clostridium innocuum | M23732 |
| Clostridium irregulare | NR_029249 |
| Clostridium isatidis | NR_026347 |
| Clostridium kluyveri | NR_074165 |
| Clostridium lactatifermentans | NR_025651 |
| Clostridium lavalense | EF564277 |
| Clostridium leptum | AJ305238 |
| Clostridium limosum | FR870444 |
| Clostridium magnum | X77835 |
| Clostridium malenominatum | FR749893 |
| Clostridium mayombei | FR733682 |
| Clostridium methylpentosum | ACEC01000059 |
| Clostridium nexile | X73443 |
| Clostridium novyi | NR_074343 |
| Clostridium orbiscindens | Y18187 |
| Clostridium oroticum | FR749922 |
| Clostridium paraputrificum | AB536771 |
| Clostridium perfringens | ABDW01000023 |
| Clostridium phytofermentans | NR_074652 |
| Clostridium piliforme | D14639 |
| Clostridium putrefaciens | NR_024995 |
| Clostridium quinii | NR_026149 |
| Clostridium ramosum | M23731 |
| Clostridium rectum | NR_029271 |
| Clostridium saccharogumia | DQ100445 |
| Clostridium saccharolyticum | CP002109 |
| Clostridium sardiniense | NR_041006 |
| Clostridium sartagoforme | NR_026490 |
| Clostridium scindens | AF262238 |
| Clostridium septicum | NR_026020 |
| Clostridium sordellii | AB448946 |
| Clostridium sp. 7_2_43FAA | ACDK01000101 |
| Clostridium sp. D5 | ADBG01000142 |
| Clostridium sp. HGF2 | AENW01000022 |
| Clostridium sp. HPB_46 | AY862516 |
| Clostridium sp. JC122 | CAEV01000127 |
| Clostridium sp. L2_50 | AAYW02000018 |
| Clostridium sp. LMG 16094 | X95274 |
| Clostridium sp. M62_1 | ACFX02000046 |
| Clostridium sp. MLG055 | AF304435 |
| Clostridium sp. MT4 E | FJ159523 |
| Clostridium sp. NMBHI_1 | JN093130 |
| Clostridium sp. NML 04A032 | EU815224 |
| Clostridium sp. SS2_1 | ABGC03000041 |
| Clostridium sp. SY8519 | AP012212 |
| Clostridium sp. TM_40 | AB249652 |
| Clostridium sp. YIT 12069 | AB491207 |
| Clostridium sp. YIT 12070 | AB491208 |
| Clostridium sphenoides | X73449 |
| Clostridium spiroforme | X73441 |
| Clostridium sporogenes | ABKW02000003 |
| Clostridium sporosphaeroides | NR_044835 |
| Clostridium stercorarium | NR_025100 |
| Clostridium sticklandii | L04167 |
| Clostridium straminisolvens | NR_024829 |
| Clostridium subterminale | NR_041795 |
| Clostridium sulfidigenes | NR_044161 |
| Clostridium symbiosum | ADLQ01000114 |
| Clostridium tertium | Y18174 |
| Clostridium tetani | NC_004557 |
| Clostridium thermocellum | NR_074629 |
| Clostridium tyrobutyricum | NR_044718 |
| Clostridium viride | NR_026204 |
| Clostridium xylanolyticum | NR_037068 |
| Collinsella aerofaciens | AAVN02000007 |
| Collinsella intestinalis | ABXH02000037 |
| Collinsella stercoris | ABXJ01000150 |
| Collinsella tanakaei | AB490807 |
| Coprobacillus cateniformis | AB030218 |
| Coprobacillus sp. 29_1 | ADKX01000057 |
| Coprobacillus sp. D7 | ACDT01000199 |
| Coprococcus catus | EU266552 |
| Coprococcus comes | ABVR01000038 |
| Coprococcus eutactus | EF031543 |
| Coprococcus sp. ART55_1 | AY350746 |
| Dialister invisus | ACIM02000001 |
| Dialister micraerophilus | AFBB01000028 |
| Dialister microaerophilus | AENT01000008 |
| Dialister pneumosintes | HM596297 |
| Dialister propionicifaciens | NR_043231 |
| Dialister sp. oral taxon 502 | GQ422739 |
| Dialister succinatiphilus | AB370249 |
| Dorea formicigenerans | AAXA02000006 |
| Dorea longicatena | AJ132842 |
| Enhydrobacter aerosaccus | ACYI01000081 |
| Enterobacter aerogenes | AJ251468 |
| Enterobacter asburiae | NR_024640 |
| Enterobacter cancerogenus | Z96078 |
| Enterobacter cloacae | FP929040 |
| Enterobacter cowanii | NR_025566 |
| Enterobacter hormaechei | AFHR01000079 |
| Enterobacter sp. 247BMC | HQ122932 |
| Enterobacter sp. 638 | NR_074777 |
| Enterobacter sp. JC163 | JN657217 |
| Enterobacter sp. SCSS | HM007811 |
| Enterobacter sp. TSE38 | HM156134 |
| Enterobacteriaceae bacterium 9_2_54FAA | ADCU01000033 |
| Enterobacteriaceae bacterium CF01Ent_1 | AJ489826 |
| Enterobacteriaceae bacterium Smarlab 3302238 | AY538694 |
| Enterococcus avium | AF133535 |
| Enterococcus caccae | AY943820 |
| Enterococcus casseliflavus | AEWT01000047 |
| Enterococcus durans | AJ276354 |
| Enterococcus faecalis | AE016830 |
| Enterococcus faecium | AM157434 |
| Enterococcus gallinarum | AB269767 |
| Enterococcus gilvus | AY033814 |
| Enterococcus hawaiiensis | AY321377 |
| Enterococcus hirae | AF061011 |
| Enterococcus italicus | AEPV01000109 |
| Enterococcus mundtii | NR_024906 |
| Enterococcus raffinosus | FN600541 |
| Enterococcus sp. BV2CASA2 | JN809766 |
| Enterococcus sp. CCRI_16620 | GU457263 |
| Enterococcus sp. F95 | FJ463817 |
| Enterococcus sp. RfL6 | AJ133478 |
| Enterococcus thailandicus | AY321376 |
| Erysipelotrichaceae bacterium 3_1_53 | ACTJ01000113 |
| Erysipelotrichaceae bacterium 5_2_54FAA | ACZW01000054 |
| Escherichia albertii | ABKX01000012 |
| Escherichia coli | NC_008563 |
| Escherichia fergusonii | CU928158 |
| Escherichia hermannii | HQ407266 |
| Escherichia sp. 1_1_43 | ACID01000033 |
| Escherichia sp. 4_1_40B | ACDM02000056 |
| Escherichia sp. B4 | EU722735 |
| Escherichia vulneris | NR_041927 |
| Eubacteriaceae bacterium P4P_50 P4 | AY207060 |
| Eubacterium barkeri | NR_044661 |
| Eubacterium biforme | ABYT01000002 |
| Eubacterium brachy | U13038 |
| Eubacterium budayi | NR_024682 |
| Eubacterium callanderi | NR_026330 |
| Eubacterium cellulosolvens | AY178842 |
| Eubacterium contortum | FR749946 |
| Eubacterium coprostanoligenes | HM037995 |
| Eubacterium cylindroides | FP929041 |
| Eubacterium desmolans | NR_044644 |
| Eubacterium dolichum | L34682 |
| Eubacterium eligens | CP001104 |
| Eubacterium fissicatena | FR749935 |
| Eubacterium hadrum | FR749933 |
| Eubacterium hallii | L34621 |
| Eubacterium infirmum | U13039 |
| Eubacterium limosum | CP002273 |
| Eubacterium moniliforme | HF558373 |
| Eubacterium multiforme | NR_024683 |
| Eubacterium nitritogenes | NR_024684 |
| Eubacterium nodatum | U13041 |
| Eubacterium ramulus | AJ011522 |
| Eubacterium rectale | FP929042 |
| Eubacterium ruminantium | NR_024661 |
| Eubacterium saburreum | AB525414 |
| Eubacterium saphenum | NR_026031 |
| Eubacterium siraeum | ABCA03000054 |
| Eubacterium sp. 3_1_31 | ACTL01000045 |
| Eubacterium sp. AS15b | HQ616364 |
| Eubacterium sp. OBRC9 | HQ616354 |
| Eubacterium sp. oral clone GI038 | AY349374 |
| Eubacterium sp. oral clone IR009 | AY349376 |
| Eubacterium sp. oral clone JH012 | AY349373 |
| Eubacterium sp. oral clone JI012 | AY349379 |
| Eubacterium sp. oral clone JN088 | AY349377 |
| Eubacterium sp. oral clone JS001 | AY349378 |
| Eubacterium sp. oral clone OH3A | AY947497 |
| Eubacterium sp. WAL 14571 | FJ687606 |
| Eubacterium tenue | M59118 |
| Eubacterium tortuosum | NR_044648 |
| Eubacterium ventriosum | L34421 |
| Eubacterium xylanophilum | L34628 |
| Eubacterium yurii | AEES01000073 |
| Fusobacterium canifelinum | AY162222 |
| Fusobacterium genomosp. C1 | AY278616 |
| Fusobacterium genomosp. C2 | AY278617 |
| Fusobacterium gonidiaformans | ACET01000043 |
| Fusobacterium mortiferum | ACDB02000034 |
| Fusobacterium naviforme | HQ223106 |
| Fusobacterium necrogenes | X55408 |
| Fusobacterium necrophorum | AM905356 |
| Fusobacterium nucleatum | ADVK01000034 |
| Fusobacterium periodonticum | ACJY01000002 |
| Fusobacterium russii | NR_044687 |
| Fusobacterium sp. 1_1_41FAA | ADGG01000053 |
| Fusobacterium sp. 11_3_2 | ACUO01000052 |
| Fusobacterium sp. 12_1B | AGWJ01000070 |
| Fusobacterium sp. 2_1_31 | ACDC02000018 |
| Fusobacterium sp. 3_1_27 | ADGF01000045 |
| Fusobacterium sp. 3_1_33 | ACQE01000178 |
| Fusobacterium sp. 3_1_36A2 | ACPU01000044 |
| Fusobacterium sp. 3_1_5R | ACDD01000078 |
| Fusobacterium sp. AC18 | HQ616357 |
| Fusobacterium sp. ACB2 | HQ616358 |
| Fusobacterium sp. AS2 | HQ616361 |
| Fusobacterium sp. CM1 | HQ616371 |
| Fusobacterium sp. CM21 | HQ616375 |
| Fusobacterium sp. CM22 | HQ616376 |
| Fusobacterium sp. D12 | ACDG02000036 |
| Fusobacterium sp. oral clone ASCF06 | AY923141 |
| Fusobacterium sp. oral clone ASCF11 | AY953256 |
| Fusobacterium ulcerans | ACDH01000090 |
| Fusobacterium varium | ACIE01000009 |
| Gemella haemolysans | ACDZ02000012 |
| Gemella morbillorum | NR_025904 |
| Gemella morbillorum | ACRX01000010 |
| Gemella sanguinis | ACRY01000057 |
| Gemella sp. oral clone ASCE02 | AY923133 |
| Gemella sp. oral clone ASCF04 | AY923139 |
| Gemella sp. oral clone ASCF12 | AY923143 |
| Gemella sp. WAL 1945J | EU427463 |
| Klebsiella oxytoca | AY292871 |
| Klebsiella pneumoniae | CP000647 |
| Klebsiella sp. AS10 | HQ616362 |
| Klebsiella sp. Co9935 | DQ068764 |
| Klebsiella sp. enrichment culture clone SRC_DSD25 | HM195210 |
| Klebsiella sp. OBRC7 | HQ616353 |
| Klebsiella sp. SP_BA | FJ999767 |
| Klebsiella sp. SRC_DSD1 | GU797254 |
| Klebsiella sp. SRC_DSD11 | GU797263 |
| Klebsiella sp. SRC_DSD12 | GU797264 |
| Klebsiella sp. SRC_DSD15 | GU797267 |
| Klebsiella sp. SRC_DSD2 | GU797253 |
| Klebsiella sp. SRC_DSD6 | GU797258 |
| Klebsiella variicola | CP001891 |
| Lachnobacterium bovis | GU324407 |
| Lachnospira multipara | FR733699 |
| Lachnospira pectinoschiza | L14675 |
| Lachnospiraceae bacterium 1_1_57FAA | ACTM01000065 |
| Lachnospiraceae bacterium 1_4_56FAA | ACTN01000028 |
| Lachnospiraceae bacterium 2_1_46FAA | ADLB01000035 |
| Lachnospiraceae bacterium 2_1_58FAA | ACTO01000052 |
| Lachnospiraceae bacterium 3_1_57FAA_CT1 | ACTP01000124 |
| Lachnospiraceae bacterium 4_1_37FAA | ADCR01000030 |
| Lachnospiraceae bacterium 5_1_57FAA | ACTR01000020 |
| Lachnospiraceae bacterium 5_1_63FAA | ACTS01000081 |
| Lachnospiraceae bacterium 6_1_63FAA | ACTV01000014 |
| Lachnospiraceae bacterium 8_1_57FAA | ACWQ01000079 |
| Lachnospiraceae bacterium 9_1_43BFAA | ACTX01000023 |
| Lachnospiraceae bacterium A4 | DQ789118 |
| Lachnospiraceae bacterium DJF VP30 | EU728771 |
| Lachnospiraceae bacterium ICM62 | HQ616401 |
| Lachnospiraceae bacterium MSX33 | HQ616384 |
| Lachnospiraceae bacterium oral taxon 107 | ADDS01000069 |
| Lachnospiraceae bacterium oral taxon F15 | HM099641 |
| Lachnospiraceae genomosp. C1 | AY278618 |
| Lactobacillus acidipiscis | NR_024718 |
| Lactobacillus acidophilus | CP000033 |
| Lactobacillus alimentarius | NR_044701 |
| Lactobacillus amylolyticus | ADNY01000006 |
| Lactobacillus amylovorus | CP002338 |
| Lactobacillus antri | ACLL01000037 |
| Lactobacillus brevis | EU194349 |
| Lactobacillus buchneri | ACGH01000101 |
| Lactobacillus casei | CP000423 |
| Lactobacillus catenaformis | M23729 |
| Lactobacillus coleohominis | ACOH01000030 |
| Lactobacillus coryniformis | NR_044705 |
| Lactobacillus crispatus | ACOG01000151 |
| Lactobacillus curvatus | NR_042437 |
| Lactobacillus delbrueckii | CP002341 |
| Lactobacillus dextrinicus | NR_036861 |
| Lactobacillus farciminis | NR_044707 |
| Lactobacillus fermentum | CP002033 |
| Lactobacillus gasseri | ACOZ01000018 |
| Lactobacillus gastricus | AICN01000060 |
| Lactobacillus genomosp. C1 | AY278619 |
| Lactobacillus genomosp. C2 | AY278620 |
| Lactobacillus helveticus | ACLM01000202 |
| Lactobacillus hilgardii | ACGP01000200 |
| Lactobacillus hominis | FR681902 |
| Lactobacillus iners | AEKJ01000002 |
| Lactobacillus jensenii | ACQD01000066 |
| Lactobacillus johnsonii | AE017198 |
| Lactobacillus kalixensis | NR_029083 |
| Lactobacillus kefiranofaciens | NR_042440 |
| Lactobacillus kefiri | NR_042230 |
| Lactobacillus kimchii | NR_025045 |
| Lactobacillus leichmannii | JX986966 |
| Lactobacillus mucosae | FR693800 |
| Lactobacillus murinus | NR_042231 |
| Lactobacillus nodensis | NR_041629 |
| Lactobacillus oeni | NR_043095 |
| Lactobacillus oris | AEKL01000077 |
| Lactobacillus parabrevis | NR_042456 |
| Lactobacillus parabuchneri | NR_041294 |
| Lactobacillus paracasei | ABQV01000067 |
| Lactobacillus parakefiri | NR_029039 |
| Lactobacillus pentosus | JN813103 |
| Lactobacillus perolens | NR_029360 |
| Lactobacillus plantarum | ACGZ02000033 |
| Lactobacillus pontis | HM218420 |
| Lactobacillus reuteri | ACGW02000012 |
| Lactobacillus rhamnosus | ABWJ01000068 |
| Lactobacillus rogosae | GU269544 |
| Lactobacillus ruminis | ACGS02000043 |
| Lactobacillus sakei | DQ989236 |
| Lactobacillus salivarius | AEBA01000145 |
| Lactobacillus saniviri | AB602569 |
| Lactobacillus senioris | AB602570 |
| Lactobacillus sp. 66c | FR681900 |
| Lactobacillus sp. BT6 | HQ616370 |
| Lactobacillus sp. KLDS 1.0701 | EU600905 |
| Lactobacillus sp. KLDS 1.0702 | EU600906 |
| Lactobacillus sp. KLDS 1.0703 | EU600907 |
| Lactobacillus sp. KLDS 1.0704 | EU600908 |
| Lactobacillus sp. KLDS 1.0705 | EU600909 |
| Lactobacillus sp. KLDS 1.0707 | EU600911 |
| Lactobacillus sp. KLDS 1.0709 | EU600913 |
| Lactobacillus sp. KLDS 1.0711 | EU600915 |
| Lactobacillus sp. KLDS 1.0712 | EU600916 |
| Lactobacillus sp. KLDS 1.0713 | EU600917 |
| Lactobacillus sp. KLDS 1.0716 | EU600921 |
| Lactobacillus sp. KLDS 1.0718 | EU600922 |
| Lactobacillus sp. KLDS 1.0719 | EU600923 |
| Lactobacillus sp. oral clone HT002 | AY349382 |
| Lactobacillus sp. oral clone HT070 | AY349383 |
| Lactobacillus sp. oral taxon 052 | GQ422710 |
| Lactobacillus tucceti | NR_042194 |
| Lactobacillus ultunensis | ACGU01000081 |
| Lactobacillus vaginalis | ACGV01000168 |
| Lactobacillus vini | NR_042196 |
| Lactobacillus vitulinus | NR_041305 |
| Lactobacillus zeae | NR_037122 |
| Lactococcus garvieae | AF061005 |
| Lactococcus lactis | CP002365 |
| Lactococcus raffinolactis | NR_044359 |
| Listeria grayi | ACCR02000003 |
| Listeria innocua | JF967625 |
| Listeria ivanovii | X56151 |
| Listeria monocytogenes | CP002003 |
| Listeria welshimeri | AM263198 |
| Megasphaera elsdenii | AY038996 |
| Megasphaera genomosp. C1 | AY278622 |
| Megasphaera genomosp. type_1 | ADGP01000010 |
| Megasphaera micronuciformis | AECS01000020 |
| Megasphaera sp. BLPYG_07 | HM990964 |
| Megasphaera sp. UPII 199_6 | AFIJ01000040 |
| Microbacterium gubbeenense | NR_025098 |
| Microbacterium lacticum | EU714351 |
| Mitsuokella jalaludinii | NR_028840 |
| Mitsuokella multacida | ABWK02000005 |
| Mitsuokella sp. oral taxon 521 | GU413658 |
| Mitsuokella sp. oral taxon G68 | GU432166 |
| Mycobacterium abscessus | AGQU01000002 |
| Mycobacterium africanum | AF480605 |
| Mycobacterium alsiensis | AJ938169 |
| Mycobacterium avium | CP000479 |
| Mycobacterium chelonae | AB548610 |
| Mycobacterium colombiense | AM062764 |
| Mycobacterium elephantis | AF385898 |
| Mycobacterium gordonae | GU142930 |
| Mycobacterium intracellulare | GQ153276 |
| Mycobacterium kansasii | AF480601 |
| Mycobacterium lacus | NR_025175 |
| Mycobacterium leprae | FM211192 |
| Mycobacterium lepromatosis | EU203590 |
| Mycobacterium mageritense | FR798914 |
| Mycobacterium mantenii | FJ042897 |
| Mycobacterium marinum | NC_010612 |
| Mycobacterium microti | NR_025234 |
| Mycobacterium neoaurum | AF268445 |
| Mycobacterium parascrofulaceum | ADNV01000350 |
| Mycobacterium paraterrae | EU919229 |
| Mycobacterium phlei | GU142920 |
| Mycobacterium seoulense | DQ536403 |
| Mycobacterium smegmatis | CP000480 |
| Mycobacterium sp. 1761 | EU703150 |
| Mycobacterium sp. 1776 | EU703152 |
| Mycobacterium sp. 1781 | EU703147 |
| Mycobacterium sp. 1791 | EU703148 |
| Mycobacterium sp. 1797 | EU703149 |
| Mycobacterium sp. AQ1GA4 | HM210417 |
| Mycobacterium sp. B10_07.09.0206 | HQ174245 |
| Mycobacterium sp. GN_10546 | FJ497243 |
| Mycobacterium sp. GN_10827 | FJ497247 |
| Mycobacterium sp. GN_11124 | FJ652846 |
| Mycobacterium sp. GN_9188 | FJ497240 |
| Mycobacterium sp. GR_2007_210 | FJ555538 |
| Mycobacterium sp. HE5 | AJ012738 |
| Mycobacterium sp. NLA001000736 | HM627011 |
| Mycobacterium sp. W | DQ437715 |
| Mycobacterium tuberculosis | CP001658 |
| Mycobacterium ulcerans | AB548725 |
| Mycobacterium vulneris | EU834055 |
| Mycoplasma agalactiae | AF010477 |
| Mycoplasma amphoriforme | AY531656 |
| Mycoplasma arthritidis | NC_011025 |
| Mycoplasma bovoculi | NR_025987 |
| Mycoplasma faucium | NR_024983 |
| Mycoplasma fermentans | CP002458 |
| Mycoplasma flocculare | X62699 |
| Mycoplasma genitalium | L43967 |
| Mycoplasma hominis | AF443616 |
| Mycoplasma orale | AY796060 |
| Mycoplasma ovipneumoniae | NR_025989 |
| Mycoplasma penetrans | NC_004432 |
| Mycoplasma pneumoniae | NC_000912 |
| Mycoplasma putrefaciens | U26055 |
| Mycoplasma salivarium | M24661 |
| Mycoplasmataceae genomo sp. P1 oral clone | DQ003614 |
| MB1_G23 | |
| Neisseria bacilliformis | AFAY01000058 |
| Neisseria cinerea | ACDY01000037 |
| Neisseria elongata | ADBF01000003 |
| Neisseria flavescens | ACQV01000025 |
| Neisseria genomo sp. P2 oral clone MB5_P15 | DQ003630 |
| Neisseria gonorrhoeae | CP002440 |
| Neisseria lactamica | ACEQ01000095 |
| Neisseria macacae | AFQE01000146 |
| Neisseria meningitidis | NC_003112 |
| Neisseria mucosa | ACDX01000110 |
| Neisseria pharyngis | AJ239281 |
| Neisseria polysaccharea | ADBE01000137 |
| Neisseria sicca | ACKO02000016 |
| Neisseria sp. KEM232 | GQ203291 |
| Neisseria sp. oral clone AP132 | AY005027 |
| Neisseria sp. oral clone JC012 | AY349388 |
| Neisseria sp. oral strain B33KA | AY005028 |
| Neisseria sp. oral taxon 014 | ADEA01000039 |
| Neisseria sp. SMC_A9199 | FJ763637 |
| Neisseria sp. TM10_1 | DQ279352 |
| Neisseria subflava | ACEO01000067 |
| Odoribacter laneus | AB490805 |
| Odoribacter splanchnicus | CP002544 |
| Oscillibacter sp. G2 | HM626173 |
| Oscillibacter valericigenes | NR_074793 |
| Oscillospira guilliermondii | AB040495 |
| Paenibacillus barcinonensis | NR_042272 |
| Paenibacillus barengoltzii | NR_042756 |
| Paenibacillus chibensis | NR_040885 |
| Paenibacillus cookii | NR_025372 |
| Paenibacillus durus | NR_037017 |
| Paenibacillus glucanolyticus | D78470 |
| Paenibacillus lactis | NR_025739 |
| Paenibacillus lautus | NR_040882 |
| Paenibacillus pabuli | NR_040853 |
| Paenibacillus polymyxa | NR_037006 |
| Paenibacillus popilliae | NR_040888 |
| Paenibacillus sp. CIP 101062 | HM212646 |
| Parabacteroides distasonis | CP000140 |
| Parabacteroides goldsteinii | AY974070 |
| Parabacteroides gordonii | AB470344 |
| Parabacteroides johnsonii | ABYH01000014 |
| Parabacteroides merdae | EU136685 |
| Parabacteroides sp. D13 | ACPW01000017 |
| Parabacteroides sp. NS31_3 | JN029805 |
| Peptococcus niger | NR_029221 |
| Peptococcus sp. oral clone JM048 | AY349389 |
| Peptococcus sp. oral taxon 167 | GQ422727 |
| Peptoniphilus asaccharolyticus | D14145 |
| Peptoniphilus duerdenii | EU526290 |
| Peptoniphilus harei | NR_026358 |
| Peptoniphilus indolicus | AY153431 |
| Peptoniphilus ivorii | Y07840 |
| Peptoniphilus lacrimalis | ADDO01000050 |
| Peptoniphilus sp. gpac007 | AM176517 |
| Peptoniphilus sp. gpac018A | AM176519 |
| Peptoniphilus sp. gpac077 | AM176527 |
| Peptoniphilus sp. gpac148 | AM176535 |
| Peptoniphilus sp. JC140 | JF824803 |
| Peptoniphilus sp. oral taxon 386 | ADCS01000031 |
| Peptoniphilus sp. oral taxon 836 | AEAA01000090 |
| Peptostreptococcaceae bacterium ph1 | JN837495 |
| Peptostreptococcus anaerobius | AY326462 |
| Peptostreptococcus micros | AM176538 |
| Peptostreptococcus sp. 9succ1 | X90471 |
| Peptostreptococcus sp. oral clone AP24 | AB175072 |
| Peptostreptococcus sp. oral clone FJ023 | AY349390 |
| Peptostreptococcus sp. P4P_31 P3 | AY207059 |
| Peptostreptococcus stomatis | ADGQ01000048 |
| Porphyromonadaceae bacterium NML 060648 | EF184292 |
| Porphyromonas asaccharolytica | AENO01000048 |
| Porphyromonas endodontalis | ACNN01000021 |
| Porphyromonas gingivalis | AE015924 |
| Porphyromonas levii | NR_025907 |
| Porphyromonas macacae | NR_025908 |
| Porphyromonas somerae | AB547667 |
| Porphyromonas sp. oral clone BB134 | AY005068 |
| Porphyromonas sp. oral clone F016 | AY005069 |
| Porphyromonas sp. oral clone P2PB_52 P1 | AY207054 |
| Porphyromonas sp. oral clone P4GB_100 P2 | AY207057 |
| Porphyromonas sp. UQD 301 | EU012301 |
| Porphyromonas uenonis | ACLR01000152 |
| Prevotella albensis | NR_025300 |
| Prevotella amnii | AB547670 |
| Prevotella bergensis | ACKS01000100 |
| Prevotella bivia | ADFO01000096 |
| Prevotella brevis | NR_041954 |
| Prevotella buccae | ACRB01000001 |
| Prevotella buccalis | JN867261 |
| Prevotella copri | ACBX02000014 |
| Prevotella corporis | L16465 |
| Prevotella dentalis | AB547678 |
| Prevotella denticola | CP002589 |
| Prevotella disiens | AEDO01000026 |
| Prevotella genomosp. C1 | AY278624 |
| Prevotella genomosp. C2 | AY278625 |
| Prevotella genomosp. P7 oral clone MB2_P31 | DQ003620 |
| Prevotella genomosp. P8 oral clone MB3_P13 | DQ003622 |
| Prevotella genomosp. P9 oral clone MB7_G16 | DQ003633 |
| Prevotella heparinolytica | GQ422742 |
| Prevotella histicola | JN867315 |
| Prevotella intermedia | AF414829 |
| Prevotella loescheii | JN867231 |
| Prevotella maculosa | AGEK01000035 |
| Prevotella marshii | AEEI01000070 |
| Prevotella melaninogenica | CP002122 |
| Prevotella micans | AGWK01000061 |
| Prevotella multiformis | AEWX01000054 |
| Prevotella multisaccharivorax | AFJE01000016 |
| Prevotella nanceiensis | JN867228 |
| Prevotella nigrescens | AFPX01000069 |
| Prevotella oralis | AEPE01000021 |
| Prevotella oris | ADDV01000091 |
| Prevotella oulorum | L16472 |
| Prevotella pallens | AFPY01000135 |
| Prevotella ruminicola | CP002006 |
| Prevotella salivae | AB108826 |
| Prevotella sp. BI_42 | AJ581354 |
| Prevotella sp. CM38 | HQ610181 |
| Prevotella sp. ICM1 | HQ616385 |
| Prevotella sp. ICM55 | HQ616399 |
| Prevotella sp. JCM 6330 | AB547699 |
| Prevotella sp. oral clone AA020 | AY005057 |
| Prevotella sp. oral clone ASCG10 | AY923148 |
| Prevotella sp. oral clone ASCG12 | DQ272511 |
| Prevotella sp. oral clone AU069 | AY005062 |
| Prevotella sp. oral clone CY006 | AY005063 |
| Prevotella sp. oral clone DA058 | AY005065 |
| Prevotella sp. oral clone FL019 | AY349392 |
| Prevotella sp. oral clone FU048 | AY349393 |
| Prevotella sp. oral clone FW035 | AY349394 |
| Prevotella sp. oral clone GI030 | AY349395 |
| Prevotella sp. oral clone GI032 | AY349396 |
| Prevotella sp. oral clone GI059 | AY349397 |
| Prevotella sp. oral clone GU027 | AY349398 |
| Prevotella sp. oral clone HF050 | AY349399 |
| Prevotella sp. oral clone ID019 | AY349400 |
| Prevotella sp. oral clone IDR_CEC_0055 | AY550997 |
| Prevotella sp. oral clone IK053 | AY349401 |
| Prevotella sp. oral clone IK062 | AY349402 |
| Prevotella sp. oral clone P4PB_83 P2 | AY207050 |
| Prevotella sp. oral taxon 292 | GQ422735 |
| Prevotella sp. oral taxon 299 | ACWZ01000026 |
| Prevotella sp. oral taxon 300 | GU409549 |
| Prevotella sp. oral taxon 302 | ACZK01000043 |
| Prevotella sp. oral taxon 310 | GQ422737 |
| Prevotella sp. oral taxon 317 | ACQH01000158 |
| Prevotella sp. oral taxon 472 | ACZS01000106 |
| Prevotella sp. oral taxon 781 | GQ422744 |
| Prevotella sp. oral taxon 782 | GQ422745 |
| Prevotella sp. oral taxon F68 | HM099652 |
| Prevotella sp. oral taxon G60 | GU432133 |
| Prevotella sp. oral taxon G70 | GU432179 |
| Prevotella sp. oral taxon G71 | GU432180 |
| Prevotella sp. SEQ053 | JN867222 |
| Prevotella sp. SEQ065 | JN867234 |
| Prevotella sp. SEQ072 | JN867238 |
| Prevotella sp. SEQ116 | JN867246 |
| Prevotella sp. SG12 | GU561343 |
| Prevotella sp. sp24 | AB003384 |
| Prevotella sp. sp34 | AB003385 |
| Prevotella stercorea | AB244774 |
| Prevotella tannerae | ACIJ02000018 |
| Prevotella timonensis | ADEF01000012 |
| Prevotella veroralis | ACVA01000027 |
| Prevotellaceae bacterium P4P_62 P1 | AY207061 |
| Propionibacteriaceae bacterium NML 02_0265 | EF599122 |
| Propionibacterium acidipropionici | NC_019395 |
| Propionibacterium acnes | ADJM01000010 |
| Propionibacterium avidum | AJ003055 |
| Propionibacterium freudenreichii | NR_036972 |
| Propionibacterium granulosum | FJ785716 |
| Propionibacterium jensenii | NR_042269 |
| Propionibacterium propionicum | NR_025277 |
| Propionibacterium sp. 434_HC2 | AFIL01000035 |
| Propionibacterium sp. H456 | AB177643 |
| Propionibacterium sp. LG | AY354921 |
| Propionibacterium sp. oral taxon 192 | GQ422728 |
| Propionibacterium sp. S555a | AB264622 |
| Propionibacterium thoenii | NR_042270 |
| Pseudomonas aeruginosa | AABQ07000001 |
| Pseudomonas fluorescens | AY622220 |
| Pseudomonas gessardii | FJ943496 |
| Pseudomonas mendocina | AAUL01000021 |
| Pseudomonas monteilii | NR_024910 |
| Pseudomonas poae | GU188951 |
| Pseudomonas pseudoalcaligenes | NR_037000 |
| Pseudomonas putida | AF094741 |
| Pseudomonas sp. 2_1_26 | ACWU01000257 |
| Pseudomonas sp. G1229 | DQ910482 |
| Pseudomonas sp. NP522b | EU723211 |
| Pseudomonas stutzeri | AM905854 |
| Pseudomonas tolaasii | AF320988 |
| Pseudomonas viridiflava | NR_042764 |
| Ralstonia pickettii | NC_010682 |
| Ralstonia sp. 5_7_47FAA | ACUF01000076 |
| Roseburia cecicola | GU233441 |
| Roseburia faecalis | AY804149 |
| Roseburia faecis | AY305310 |
| Roseburia hominis | AJ270482 |
| Roseburia intestinalis | FP929050 |
| Roseburia inulinivorans | AJ270473 |
| Roseburia sp. 11SE37 | FM954975 |
| Roseburia sp. 11SE38 | FM954976 |
| Rothia aeria | DQ673320 |
| Rothia dentocariosa | ADDW01000024 |
| Rothia mucilaginosa | ACVO01000020 |
| Rothia nasimurium | NR_025310 |
| Rothia sp. oral taxon 188 | GU470892 |
| Ruminobacter amylophilus | NR_026450 |
| Ruminococcaceae bacterium D16 | ADDX01000083 |
| Ruminococcus albus | AY445600 |
| Ruminococcus bromii | EU266549 |
| Ruminococcus callidus | NR_029160 |
| Ruminococcus champanellensis | FP929052 |
| Ruminococcus flavefaciens | NR_025931 |
| Ruminococcus gnavus | X94967 |
| Ruminococcus hansenii | M59114 |
| Ruminococcus lactaris | ABOU02000049 |
| Ruminococcus obeum | AY169419 |
| Ruminococcus sp. 18P13 | AJ515913 |
| Ruminococcus sp. 5_1_39BFAA | ACII01000172 |
| Ruminococcus sp. 9SE51 | FM954974 |
| Ruminococcus sp. ID8 | AY960564 |
| Ruminococcus sp. K_1 | AB222208 |
| Ruminococcus torques | AAVP02000002 |
| Salmonella bongori | NR_041699 |
| Salmonella enterica | NC_011149 |
| Salmonella enterica | NC_011205 |
| Salmonella enterica | DQ344532 |
| Salmonella enterica | ABEH02000004 |
| Salmonella enterica | ABAK02000001 |
| Salmonella enterica | NC_011080 |
| Salmonella enterica | EU118094 |
| Salmonella enterica | NC_011094 |
| Salmonella enterica | AE014613 |
| Salmonella enterica | ABFH02000001 |
| Salmonella enterica | ABEM01000001 |
| Salmonella enterica | ABAM02000001 |
| Salmonella typhimurium | DQ344533 |
| Salmonella typhimurium | AF170176 |
| Selenomonas artemidis | HM596274 |
| Selenomonas dianae | GQ422719 |
| Selenomonas flueggei | AF287803 |
| Selenomonas genomosp. C1 | AY278627 |
| Selenomonas genomosp. C2 | AY278628 |
| Selenomonas genomosp. P5 | AY341820 |
| Selenomonas genomosp. P6 oral clone MB3_C41 | DQ003636 |
| Selenomonas genomosp. P7 oral clone MB5_C08 | DQ003627 |
| Selenomonas genomosp. P8 oral clone MB5_P06 | DQ003628 |
| Selenomonas infelix | AF287802 |
| Selenomonas noxia | GU470909 |
| Selenomonas ruminantium | NR_075026 |
| Selenomonas sp. FOBRC9 | HQ616378 |
| Selenomonas sp. oral clone FT050 | AY349403 |
| Selenomonas sp. oral clone GI064 | AY349404 |
| Selenomonas sp. oral clone GT010 | AY349405 |
| Selenomonas sp. oral clone HU051 | AY349406 |
| Selenomonas sp. oral clone IK004 | AY349407 |
| Selenomonas sp. oral clone IQ048 | AY349408 |
| Selenomonas sp. oral clone JI021 | AY349409 |
| Selenomonas sp. oral clone JS031 | AY349410 |
| Selenomonas sp. oral clone OH4A | AY947498 |
| Selenomonas sp. oral clone P2PA_80 P4 | AY207052 |
| Selenomonas sp. oral taxon 137 | AENV01000007 |
| Selenomonas sp. oral taxon 149 | AEEJ01000007 |
| Selenomonas sputigena | ACKP02000033 |
| Serratia fonticola | NR_025339 |
| Serratia liquefaciens | NR_042062 |
| Serratia marcescens | GU826157 |
| Serratia odorifera | ADBY01000001 |
| Serratia proteamaculans | AAUN01000015 |
| Shigella boydii | AAKA01000007 |
| Shigella dysenteriae | NC_007606 |
| Shigella flexneri | AE005674 |
| Shigella sonnei | NC_007384 |
| Sphingobacterium faecium | NR_025537 |
| Sphingobacterium mizutaii | JF708889 |
| Sphingobacterium multivorum | NR_040953 |
| Sphingobacterium spiritivorum | ACHA02000013 |
| Sphingomonas echinoides | NR_024700 |
| Sphingomonas sp. oral clone FI012 | AY349411 |
| Sphingomonas sp. oral clone FZ016 | AY349412 |
| Sphingomonas sp. oral taxon A09 | HM099639 |
| Sphingomonas sp. oral taxon F71 | HM099645 |
| Staphylococcaceae bacterium NML 92_0017 | AY841362 |
| Staphylococcus aureus | CP002643 |
| Staphylococcus auricularis | JQ624774 |
| Staphylococcus capitis | ACFR01000029 |
| Staphylococcus caprae | ACRH01000033 |
| Staphylococcus carnosus | NR_075003 |
| Staphylococcus cohnii | JN175375 |
| Staphylococcus condimenti | NR_029345 |
| Staphylococcus epidermidis | ACHE01000056 |
| Staphylococcus equorum | NR_027520 |
| Staphylococcus fleurettii | NR_041326 |
| Staphylococcus haemolyticus | NC_007168 |
| Staphylococcus hominis | AM157418 |
| Staphylococcus lugdunensis | AEQA01000024 |
| Staphylococcus pasteuri | FJ189773 |
| Staphylococcus pseudintermedius | CP002439 |
| Staphylococcus saccharolyticus | NR_029158 |
| Staphylococcus saprophyticus | NC_007350 |
| Staphylococcus sciuri | NR_025520 |
| Staphylococcus sp. clone bottae7 | AF467424 |
| Staphylococcus sp. H292 | AB177642 |
| Staphylococcus sp. H780 | AB177644 |
| Staphylococcus succinus | NR_028667 |
| Staphylococcus vitulinus | NR_024670 |
| Staphylococcus warneri | ACPZ01000009 |
| Staphylococcus xylosus | AY395016 |
| Streptobacillus moniliformis | NR_027615 |
| Streptococcus agalactiae | AAJO01000130 |
| Streptococcus alactolyticus | NR_041781 |
| Streptococcus anginosus | AECT01000011 |
| Streptococcus australis | AEQR01000024 |
| Streptococcus bovis | AEEL01000030 |
| Streptococcus canis | AJ413203 |
| Streptococcus constellatus | AY277942 |
| Streptococcus cristatus | AEVC01000028 |
| Streptococcus downei | AEKN01000002 |
| Streptococcus dysgalactiae | AP010935 |
| Streptococcus equi | CP001129 |
| Streptococcus equinus | AEVB01000043 |
| Streptococcus gallolyticus | FR824043 |
| Streptococcus genomosp. C1 | AY278629 |
| Streptococcus genomosp. C2 | AY278630 |
| Streptococcus genomosp. C3 | AY278631 |
| Streptococcus genomosp. C4 | AY278632 |
| Streptococcus genomosp. C5 | AY278633 |
| Streptococcus genomosp. C6 | AY278634 |
| Streptococcus genomosp. C7 | AY278635 |
| Streptococcus genomosp. C8 | AY278609 |
| Streptococcus gordonii | NC_009785 |
| Streptococcus infantarius | ABJK02000017 |
| Streptococcus infantis | AFNN01000024 |
| Streptococcus intermedius | NR_028736 |
| Streptococcus lutetiensis | NR_037096 |
| Streptococcus massiliensis | AY769997 |
| Streptococcus milleri | X81023 |
| Streptococcus mitis | AM157420 |
| Streptococcus mutans | AP010655 |
| Streptococcus oligofermentans | AY099095 |
| Streptococcus oralis | ADMV01000001 |
| Streptococcus parasanguinis | AEKM01000012 |
| Streptococcus pasteurianus | AP012054 |
| Streptococcus peroris | AEVF01000016 |
| Streptococcus pneumoniae | AE008537 |
| Streptococcus porcinus | EF121439 |
| Streptococcus pseudopneumoniae | FJ827123 |
| Streptococcus pseudoporcinus | AENS01000003 |
| Streptococcus pyogenes | AE006496 |
| Streptococcus ratti | X58304 |
| Streptococcus salivarius | AGBV01000001 |
| Streptococcus sanguinis | NR_074974 |
| Streptococcus sinensis | AF432857 |
| Streptococcus sp. 16362 | JN590019 |
| Streptococcus sp. 2_1_36FAA | ACOI01000028 |
| Streptococcus sp. 2285_97 | AJ131965 |
| Streptococcus sp. 69130 | X78825 |
| Streptococcus sp. AC15 | HQ616356 |
| Streptococcus sp. ACS2 | HQ616360 |
| Streptococcus sp. AS20 | HQ616366 |
| Streptococcus sp. BS35a | HQ616369 |
| Streptococcus sp. C150 | ACRI01000045 |
| Streptococcus sp. CM6 | HQ616372 |
| Streptococcus sp. CM7 | HQ616373 |
| Streptococcus sp. ICM10 | HQ616389 |
| Streptococcus sp. ICM12 | HQ616390 |
| Streptococcus sp. ICM2 | HQ616386 |
| Streptococcus sp. ICM4 | HQ616387 |
| Streptococcus sp. ICM45 | HQ616394 |
| Streptococcus sp. M143 | ACRK01000025 |
| Streptococcus sp. M334 | ACRL01000052 |
| Streptococcus sp. OBRC6 | HQ616352 |
| Streptococcus sp. oral clone ASB02 | AY923121 |
| Streptococcus sp. oral clone ASCA03 | DQ272504 |
| Streptococcus sp. oral clone ASCA04 | AY923116 |
| Streptococcus sp. oral clone ASCA09 | AY923119 |
| Streptococcus sp. oral clone ASCB04 | AY923123 |
| Streptococcus sp. oral clone ASCB06 | AY923124 |
| Streptococcus sp. oral clone ASCC04 | AY923127 |
| Streptococcus sp. oral clone ASCC05 | AY923128 |
| Streptococcus sp. oral clone ASCC12 | DQ272507 |
| Streptococcus sp. oral clone ASCD01 | AY923129 |
| Streptococcus sp. oral clone ASCD09 | AY923130 |
| Streptococcus sp. oral clone ASCD10 | DQ272509 |
| Streptococcus sp. oral clone ASCE03 | AY923134 |
| Streptococcus sp. oral clone ASCE04 | AY953253 |
| Streptococcus sp. oral clone ASCE05 | DQ272510 |
| Streptococcus sp. oral clone ASCE06 | AY923135 |
| Streptococcus sp. oral clone ASCE09 | AY923136 |
| Streptococcus sp. oral clone ASCE10 | AY923137 |
| Streptococcus sp. oral clone ASCE12 | AY923138 |
| Streptococcus sp. oral clone ASCF05 | AY923140 |
| Streptococcus sp. oral clone ASCF07 | AY953255 |
| Streptococcus sp. oral clone ASCF09 | AY923142 |
| Streptococcus sp. oral clone ASCG04 | AY923145 |
| Streptococcus sp. oral clone BW009 | AY005042 |
| Streptococcus sp. oral clone CH016 | AY005044 |
| Streptococcus sp. oral clone GK051 | AY349413 |
| Streptococcus sp. oral clone GM006 | AY349414 |
| Streptococcus sp. oral clone P2PA_41 P2 | AY207051 |
| Streptococcus sp. oral clone P4PA_30 P4 | AY207064 |
| Streptococcus sp. oral taxon 071 | AEEP01000019 |
| Streptococcus sp. oral taxon G59 | GU432132 |
| Streptococcus sp. oral taxon G62 | GU432146 |
| Streptococcus sp. oral taxon G63 | GU432150 |
| Streptococcus sp. SHV515 | Y07601 |
| Streptococcus suis | FM252032 |
| Streptococcus thermophilus | CP000419 |
| Streptococcus uberis | HQ391900 |
| Streptococcus urinalis | DQ303194 |
| Streptococcus vestibularis | AEKO01000008 |
| Streptococcus viridans | AF076036 |
| Sutterella morbirenis | AJ832129 |
| Sutterella parvirubra | AB300989 |
| Sutterella sanguinus | AJ748647 |
| Sutterella sp. YIT 12072 | AB491210 |
| Sutterella stercoricanis | NR_025600 |
| Sutterella wadsworthensis | ADMF01000048 |
| Synergistes genomosp. C1 | AY278615 |
| Synergistes sp. RMA 14551 | DQ412722 |
| Synergistetes bacterium ADV897 | GQ258968 |
| Synergistetes bacterium LBVCM1157 | GQ258969 |
| Synergistetes bacterium oral taxon 362 | GU410752 |
| Synergistetes bacterium oral taxon D48 | GU430992 |
| Turicibacter sanguinis | AF349724 |
| Veillonella atypica | AEDS01000059 |
| Veillonella dispar | ACIK02000021 |
| Veillonella genomosp. P1 oral clone MB5_P17 | DQ003631 |
| Veillonella montpellierensis | AF473836 |
| Veillonella parvula | ADFU01000009 |
| Veillonella sp. 3_1_44 | ADCV01000019 |
| Veillonella sp. 6_1_27 | ADCW01000016 |
| Veillonella sp. ACP1 | HQ616359 |
| Veillonella sp. AS16 | HQ616365 |
| Veillonella sp. BS32b | HQ616368 |
| Veillonella sp. ICM51a | HQ616396 |
| Veillonella sp. MSA12 | HQ616381 |
| Veillonella sp. NVG 100cf | EF108443 |
| Veillonella sp. OK11 | JN695650 |
| Veillonella sp. oral clone ASCA08 | AY923118 |
| Veillonella sp. oral clone ASCB03 | AY923122 |
| Veillonella sp. oral clone ASCG01 | AY923144 |
| Veillonella sp. oral clone ASCG02 | AY953257 |
| Veillonella sp. oral clone OH1A | AY947495 |
| Veillonella sp. oral taxon 158 | AENU01000007 |
| Veillonellaceae bacterium oral taxon 131 | GU402916 |
| Veillonellaceae bacterium oral taxon 155 | GU470897 |
| Vibrio cholerae | AAUR01000095 |
| Vibrio fluvialis | X76335 |
| Vibrio furnissii | CP002377 |
| Vibrio mimicus | ADAF01000001 |
| Vibrio parahaemolyticus | AAWQ01000116 |
| Vibrio sp. RC341 | ACZT01000024 |
| Vibrio vulnificus | AE016796 |
| Yersinia aldovae | AJ871363 |
| Yersinia aleksiciae | AJ627597 |
| Yersinia bercovieri | AF366377 |
| Yersinia enterocolitica | FR729477 |
| Yersinia frederiksenii | AF366379 |
| Yersinia intermedia | AF366380 |
| Yersinia kristensenii | ACCA01000078 |
| Yersinia mollaretii | NR_027546 |
| Yersinia pestis | AE013632 |
| Yersinia pseudotuberculosis | NC_009708 |
| Yersinia rohdei | ACCD01000071 |

TABLE 3
Exemplary Bacterial Strains

| Strain | Deposit Number |
|---|---|
| Parabacteroides goldsteinii | PTA-126574 |
| Bifidobacterium animalis ssp. lactis Strain A | PTA-125097 |
| Blautia Massiliensis Strain A | PTA-125134 |
| Prevotella Strain B | NRRL accession |
| | Number B 50329 |
| Prevotella Histicola | PTA-126140 |
| Blautia Strain A | PTA-125346 |
| Lactococcus lactis cremoris Strain A | PTA-125368 |
| Lactobacillus salivarius | PTA-125893 |
| Ruminococcus gnavus strain | PTA-125706 |
| Tyzzerella nexilis strain | PTA-125707 |
| Paraclostridium benzoelyticum | PTA-125894 |
| Ruminococcus gnavus (also referred to as | PTA-126695 |
| Mediterraneibacter gnavus) | |
| Veillonella parvula | PTA-125710 |
| Veillonella atypica Strain A | PTA-125709 |
| Veillonella atypica Strain B | PTA-125711 |
| Veillonella parvula Strain A | PTA-125691 |
| Veillonella parvula Strain B | PTA-125711 |
| Veillonella tobetsuensis Strain A | PTA-125708 |
| Agathobaculum sp. | PTA-125892 |
| Turicibacter sanguinis | PTA-125889 |
| Klebsiella quasipneumoniae subsp. | PTA-125891 |
| similipneumoniae | |
| Klebsiella oxytoca | PTA-125890 |
| Megasphaera Sp. Strain A | PTA-126770 |
| Megasphaera Sp. | PTA-126837 |
| Harryflintia acetispora | PTA-126694 |
| Fournierella massiliensis | PTA-126696 |

TABLE 4
Exemplary Bacterial Strains

| | |
|---|---|
| Escherichia coli | NCIMB 12210 |
| Enterococcus faecalis | NCIMB 13280 |
| Bacteroides fragilis | DSM 2151 |
| Bacteroides vulgatus | DSM 1447 |
| Bacteroides ovatus | DSM 1896 |
| Megasphaera massiliensis | DSM 26228 |
| Megasphaera elsdenii | NCIMB 8927 |
| Megasphaera massiliensis | NCIMB 42787 |
| Bifidobacterium breve | DSM 20213 |
| Bifidobacterium longum subsp. longum | DSM 20219 |
| Faecalibacterium prausnitzii | DSM17677 |
| Anaerostipes hadrus | DSM 3319 |
| Blautia coccoides | DSM 935 |
| Dorea longicatena | DSM 13814 |
| Parabacteroides distasonis | DSM 20701 |
| Faecalicatena contorta | DSM3982 |
| Ruminococcus gnavus | ATCC29149 |
| Megasphaera massiliensis | NCIMB 43388 |
| Megasphaera massiliensis | NCIMB 43389 |
| Megasphaera spp. | NCIMB 43385 |
| Megasphaera spp. | NCIMB 43386 |
| Megasphaera spp. | NCIMB 43387 |
| Parabacteroides distasonis (also referred to as | NCIMB 42382 |
| “Parabacteroides sp 755”) | |
| Bacillus amyloliquefaciens | NCIMB 43088 |
| Bacillus amyloliquefaciens | NCIMB 43087 |
| Bacillus amyloliquefaciens | NCIMB 43086 |

### Modified Bacteria

In some aspects, the bacteria described herein are modified such that they comprise, are linked to, and/or are bound by a therapeutic moiety.

In some embodiments, the therapeutic moiety is a cancer-specific moiety. In some embodiments, the cancer-specific moiety has binding specificity for a cancer cell (e.g., has binding specificity for a cancer-specific antigen). In some embodiments, the cancer-specific moiety comprises an antibody or antigen binding fragment thereof. In some embodiments, the cancer-specific moiety comprises a T cell receptor or a chimeric antigen receptor (CAR). In some embodiments, the cancer-specific moiety comprises a ligand for a receptor expressed on the surface of a cancer cell or a receptor-binding fragment thereof. In some embodiments, the cancer-specific moiety is a bipartite fusion protein that has two parts: a first part that binds to and/or is linked to the bacterium and a second part that is capable of binding to a cancer cell (e.g., by having binding specificity for a cancer-specific antigen). In some embodiments, the first part is a fragment of or a full-length peptidoglycan recognition protein, such as PGRP. In some embodiments the first part has binding specificity for the bacteria (e.g., by having binding specificity for a bacterial antigen). In some embodiments, the first and/or second part comprises an antibody or antigen binding fragment thereof. In some embodiments, the first and/or second part comprises a T cell receptor or a chimeric antigen receptor (CAR). In some embodiments, the first and/or second part comprises a ligand for a receptor expressed on the surface of a cancer cell or a receptor-binding fragment thereof. In certain embodiments, co-administration of the cancer-specific moiety with the pharmaceutical agent (either in combination or in separate administrations) increases the targeting of the pharmaceutical agent to the cancer cells.

In some embodiments, the bacteria described herein can be modified such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of a bacteria-binding moiety that binds to the bacteria. In some embodiments, the bacteria-binding moiety is a fragment of or a full-length peptidoglycan recognition protein, such as PGRP. In some embodiments the bacteria-binding moiety has binding specificity for the bacteria (e.g., by having binding specificity for a bacterial antigen). In some embodiments, the bacteria-binding moiety comprises an antibody or antigen binding fragment thereof. In some embodiments, the bacteria-binding moiety comprises a T cell receptor or a chimeric antigen receptor (CAR). In some embodiments, the bacteria-binding moiety comprises a ligand for a receptor expressed on the surface of a cancer cell or a receptor-binding fragment thereof. In certain embodiments, co-administration of the magnetic and/or paramagnetic moiety with the bacteria (either together or in separate administrations) can be used to increase the targeting of the bacteria (e.g., to cancer cells and/or a part of a subject where cancer cells are present.

### Pharmaceutical Agents and Compositions

In certain embodiments, provided herein are pharmaceutical compositions and/or solid dosage forms (e.g., pharmaceutical products having a solid dosage form) comprising a pharmaceutical agent that contains bacteria. The bacteria can be live bacteria; non-live (dead) bacteria; non replicating bacteria; gamma irradiated bacteria; and/or lyophilized bacteria.

In certain embodiments, provided herein are pharmaceutical agents that contain bacteria. In some embodiments, the pharmaceutical agent can optionally contain one or more additional components, such as a cryoprotectant. The pharmaceutical agent can be lyophilized (e.g., resulting in a powder). The pharmaceutical agent can be combined with one or more excipients (e.g., pharmaceutically acceptable excipients) in the pharmaceutical composition and/or solid dosage form (e.g., solid dose form).

In some embodiments, the pharmaceutical agent has a fine and smooth granulated powder appearance.

In some embodiments, the pharmaceutical agent has an off-white to brown, fine powder appearance.

In certain embodiments, provided herein are pharmaceutical compositions and/or solid dosage forms comprising a pharmaceutical agent that contains bacteria. The bacteria can be live bacteria The bacteria can be live bacteria; non-live (dead) bacteria; non replicating bacteria; gamma irradiated bacteria; and/or lyophilized bacteria.

In some embodiments, the pharmaceutical agents comprise bacteria (e.g., whole bacteria) (e.g., live bacteria, killed bacteria, attenuated bacteria). In some embodiments, the pharmaceutical agents comprise bacteria from one or more (e.g., 1, 2, 3, 4, 5, 6, 7, 8, 9, 10, or more) of the bacteria strains or species or taxonomic groups listed herein. In some embodiments, the pharmaceutical agents comprise bacteria from one of the bacteria strains or species or taxonomic groups listed herein. In some embodiments, the pharmaceutical agents comprise freeze dried (e.g., lyophilized) bacteria. In some embodiments, the pharmaceutical agent comprises gamma irradiated bacteria. In some embodiments, the pharmaceutical agents comprise bacteria from one of the bacteria strains or species described herein, e.g., Lactococcus, Prevotella, Bifidobacterium, Veillonella, Fournierella, Harryflintia, Megasphaera; e.g., Lactococcus lactis cremoris; Prevotella histicola; Bifidobacterium animalis lactis; Veillonella parvula; Fournierella massiliensis; Harryflintia acetispora; or Megasphaera sp.

In some embodiments, to quantify the numbers of bacteria present in a sample, electron microscopy (e.g., EM of ultrathin frozen sections) can be used to visualize the bacteria and count their relative numbers. Alternatively, nanoparticle tracking analysis (NTA), Coulter counting, or dynamic light scattering (DLS) or a combination of these techniques can be used. NTA and the Coulter counter count particles and show their sizes. DLS gives the size distribution of particles, but not the concentration. Bacteria frequently have diameters of 1-2 um (microns). The full range is 0.2-20 um. Combined results from Coulter counting and NTA can reveal the numbers of bacteria in a given sample. Coulter counting reveals the numbers of particles with diameters of 0.7-10 um. For most bacterial samples, the Coulter counter alone can reveal the number of bacteria in a sample. For NTA, a Nanosight instrument can be obtained from Malvem Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm-3 um.

In some embodiments, the bacteria may be quantified based on total cell count (TCC), e.g, by Coulter counter.

In some embodiments, the bacteria may be quantified based on particle count. For example, total particle count of a bacteria preparation can be measured using NTA.

In some embodiments, the bacteria may be quantified based on the amount of protein, lipid, or carbohydrate. For example, total protein content of a bacteria and/or preparation can be measured using the Bradford assay or BCA.

In some embodiments, bacteria are isolated away from one or more other bacterial components of the source bacterial culture. In some embodiments, the pharmaceutical agent further comprises other bacterial components.

In certain aspects, provided herein are pharmaceutical agents, pharmaceutical compositions, and/or solid dosage forms that comprise bacteria useful for the treatment and/or prevention of disease (e.g., a cancer, an autoimmune disease, an inflammatory disease, a metabolic disease, or a dysbiosis), as well as methods of making and/or identifying such bacteria, and methods of using pharmaceutical agents, pharmaceutical compositions and solid dosage forms thereof (e.g., for the treatment of a cancer, an autoimmune disease, an inflammatory disease, or a metabolic disease, either alone or in combination with other therapeutics). In some embodiments, the pharmaceutical agents comprise bacteria (e.g., whole bacteria) (e.g., live bacteria, dead (e.g., killed) bacteria, non-replicating bacteria, attenuated bacteria). In some embodiments, the pharmaceutical agents comprise bacteria from one or more of the bacteria strains or species or taxonomic groups listed herein. In some embodiments, the pharmaceutical agents bacteria from one of the bacteria strains or species or taxonomic groups listed herein. In some embodiments, the pharmaceutical agents comprise bacteria from one of the bacteria strains or species described herein, e.g., Lactococcus, Prevotella, Bifidobacterium, Veillonella, Fournierella, Harryflintia, Megasphaera; e.g., Lactococcus lactis cremoris; Prevotella histicola; Bifidobacterium animalis lactis; Veillonella parvula; Fournierella massiliensis; Harryflintia acetispora; or Megasphaera sp.

In certain aspects, provided are pharmaceutical agents, pharmaceutical compositions, and/or solid dosage forms for administration to a subject (e.g., human subject). In some embodiments, the pharmaceutical agents, pharmaceutical compositions, and/or solid dosage forms are combined with additional active and/or inactive materials in order to produce a final product, which may be in single dosage unit or in a multi-dose format. In some embodiments, the pharmaceutical agent is combined with an adjuvant such as an immuno-adjuvant (e.g., a STING agonist, a TLR agonist, or a NOD agonist).

In some embodiments, the pharmaceutical agent, pharmaceutical composition and/or solid dosage form comprises at least one carbohydrate.

In some embodiments, the pharmaceutical agent, pharmaceutical composition and/or solid dosage form comprises at least one lipid. In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the pharmaceutical agent, pharmaceutical composition and/or solid dosage form comprises at least one mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the pharmaceutical agent, pharmaceutical composition and/or solid dosage form comprises at least one vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the pharmaceutical agent, pharmaceutical composition and/or solid dosage form comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

Suitable excipients that can be included in the pharmaceutical agent, pharmaceutical composition and/or solid dosage form can be one or more pharmaceutically acceptable excipients known in the art. For example, see Rowe, Sheskey, and Quinn, eds., Handbook of Pharmaceutical Excipients, sixth ed.; 2009; Pharmaceutical Press and American Pharmacists Association.

### Solid Dosage Forms

The pharmaceutical agent can be formulated into a solid dosage form. The solid dosage form described herein can be, e.g., a tablet or a minitablet. Further, a plurality of minitablets can be in (e.g., loaded into) a capsule.

In certain embodiments, the solid dosage form comprises a capsule. In some embodiments, the capsule is a size 00, size 0, size 1, size 2, size 3, size 4, or size 5 capsule. In some embodiments, the capsule is a size 0 capsule. As used herein, the size of the capsule refers to the size of the tablet prior to application of an enteric coating. In some embodiments, the capsule is banded after loading (and prior to enterically coating the capsule). In some embodiments, the capsule is banded with an HPMC-based banding solution.

In some embodiments, the solid dosage form comprises a tablet (>4 mm) (e.g., 5 mm-17 mm). For example, the tablet is a 5 mm, 6 mm, 7 mm, 8 mm, 9 mm, 10 mm, 11 mm, 12 mm, 13 mm, 14 mm, 15 mm, 16 mm, 17 mm, or 18 mm tablet. The size refers to the diameter of the tablet, as is known in the art. As used herein, the size of the tablet refers to the size of the tablet prior to application of an enteric coating.

In some embodiments, the solid dosage form comprises a minitablet. The minitablet can be in the size range of 1 mm-4 mm range. E.g., the minitablet can be a 1 mm minitablet, 1.5 mm minitablet, 2 mm minitablet, 3 mm minitablet, or 4 mm minitablet. The size refers to the diameter of the minitablet, as is known in the art. As used herein, the size of the minitablet refers to the size of the minitablet prior to application of an enteric coating.

The minitablets can be in a capsule. The capsule can be a size 00, size 0, size 1, size 2, size 3, size 4, or size 5 capsule. The capsule that contains the minitablets can comprise HPMC (hydroxyl propyl methyl cellulose) or gelatin. The minitablets can be inside a capsule: the number of minitablets inside a capsule will depend on the size of the capsule and the size of the minitablets. As an example, a size 0 capsule can contain 31-35 (an average of 33) minitablets that are 3 mm minitablets. In some embodiments, the capsule is banded after loading. In some embodiments, the capsule is banded with an HPMC-based banding solution.

### Coating

The solid dosage form (e.g., tablet or minitablet) described herein can be enterically coated, e.g., with one enteric coating layer or with two layers of enteric coating, e.g., an inner enteric coating and an outer enteric coating. The inner enteric coating and outer enteric coating are not identical (e.g., the inner enteric coating and outer enteric coating do not contain the same components in the same amounts). The enteric coating allows for release of the pharmaceutical agent, e.g., in the small intestine, e.g., upper small intestine, e.g., duodenum and/or jejunum.

Release of the pharmaceutical agent in the small intestine, e.g., in the upper small intestine, e.g., in the duodenum, or in the jejunum, allows the pharmaceutical agent to target and affect cells (e.g., epithelial cells and/or immune cells) located at these specific locations, e.g., which can cause a local effect in the small intestine and/or cause a systemic effect (e.g., an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives.

Examples of other materials that can be used in the enteric coating (e.g., the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleurtic acid), plastics, plant fibers, zein, AQUA-ZEIN® (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

The enteric coating (e.g., the one enteric coating or the inner enteric coating and/or the outer enteric coating) can include a methacrylic acid ethyl acrylate (MAE) copolymer (1:1).

The one enteric coating can include methacrylic acid ethyl acrylate (MAE) copolymer (1:1) (such as Kollicoat MAE 100P).

The one enteric coating can include a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

Other examples of materials that can be used in the enteric coating (e.g., the one enteric coating or the inner enteric coating and/or the outer enteric coating) include those described in, e.g., U.S. Pat. Nos. 6,312,728; 6,623,759; 4,775,536; 5,047,258; 5,292,522; 6,555,124; 6,638,534; U.S. 2006/0210631; U.S. 2008/200482; U.S. 2005/0271778; U.S. 2004/0028737; WO 2005/044240.

See also, e.g., U.S. Pat. No. 9,233,074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate; suitable methacrylic acid copolymers include: poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name; partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit S100 trade name.

In certain aspects, the solid dosage form (e.g., tablet or minitablet) described herein further comprises a sub-coating. In some embodiments, the solid dosage form comprises a sub-coating, e.g., in addition to the enteric coating, e.g., the sub-coating is beneath the enteric coating (e.g., between the solid dosage form and the enteric coating). In some embodiments, the sub-coating comprises Opadry QX, e.g., Opadry QX Blue. The sub-coat can be used, e.g., to visually mask the appearance of the therapeutic agent.

### Dose

The dose of the pharmaceutical agent (e.g., for human subjects) is the dose per solid dosage form, such as per capsule or tablet or per total number of minitablets used in a capsule.

In embodiments where dose is determined by total cell count, total cell count can be determined by Coulter counter.

In some embodiments, the pharmaceutical agent comprises bacteria and the dose of bacteria is about 1×10⁷ to about 2×10¹² (e.g., about 3×10¹⁰ or about 1.5×10¹¹ or about 1.5×10¹²) cells (e.g., wherein cell number is determined by total cell count, which is determined by Coulter counter), wherein the dose is per capsule or tablet or per total number of minitablets in a capsule. In some embodiments, the pharmaceutical agent comprises bacteria and the dose of bacteria is about 1×10¹⁰ to about 2×10¹² (e.g., about 1.6×10¹¹ or about 8×10¹¹ or about 9.6×10¹¹ about 12.8×10¹¹ or about 1.6×10¹²) cells (e.g., wherein cell number is determined by total cell count, which is determined by Coulter counter), wherein the dose is per capsule or tablet or per total number of minitablets in a capsule.

In some embodiments, the pharmaceutical agent comprises bacteria and the dose of bacteria is about 1×10⁹, about 3×10⁹, about 5×10⁹, about 1.5×10¹⁰, about 3×10¹⁰, about 5×10¹⁰, about 1.5×10¹¹, about 1.5×10¹², or about 2×10² cells, wherein the dose is per capsule or tablet or per total number of minitablets in a capsule.

In some embodiments, the pharmaceutical agent dose can be a milligram (mg) dose determined by weight the pharmaceutical agent (e.g., a powder comprising bacteria). The dose of the pharmaceutical agent is per capsule or tablet or per total number of minitablets, e.g., in a capsule.

For example, to administer a 1× dose of the pharmaceutical agent of about 400 mg, about 200 mg of the pharmaceutical agent is present per capsule and two capsules are administered, resulting in a dose of about 400 mg. The two capsules can be administered, for example, 1× or 2× daily.

For example, for a minitablet: about 0.1 to about 3.5 mg (0.1, 0.35, 1.0, 3.5 mg) of the pharmaceutical agent can be contained per minitablet. The minitablets can be inside a capsule: the number of minitablets inside a capsule will depend on the size of the capsule and the size of the minitablets. For example, an average of 33 (range of 31-35) 3 mm minitablets fit inside a size 0 capsule. As an example, 0.1-3.5 mg of the pharmaceutical agent per minitablet, the dose range will be 3.3 mg-115.5 mg (for 33 minitablets in size 0 capsule) per capsule (3.1 mg-108.5 mg for 31 minitablets in size 0 capsule) (3.5 mg-122.5 mg for 35 minitablets in size 0 capsule). Multiple capsules and/or larger capsule(s) can be administered to increase the administered dose and/or can be administered one or more times per day to increase the administered dose.

In some embodiments, the dose can be about 3 mg to about 125 mg of the pharmaceutical agent, per capsule or tablet or per total number of minitablets, e.g., in a capsule.

In some embodiments, the dose can be about 35 mg to about 1200 mg (e.g., about 35 mg, about 125 mg, about 350 mg, or about 1200 mg) of the pharmaceutical agent.

In some embodiments, the dose of the pharmaceutical agent can be about 30 mg to about 3500 mg (about 25, about 50, about 75, about 100, about 150, about 250, about 300, about 350, about 400, about 500, about 600, about 750, about 1000, about 1250, about 1300, about 2000, about 2500, about 3000, or about 3500 mg).

A human dose can be calculated appropriately based on allometric scaling of a dose administered to a model organism (e.g., mouse).

In some embodiments, one or two tablets capsules can be administered one or two times a day.

The pharmaceutical agent contains the bacteria, or contains a powder comprising bacteria, and can also contain one or more additional components, such as a cryoprotectant.

In some embodiments, the mg (by weight) dose of the pharmaceutical agent is, e.g., about 1 mg to about 500 mg per capsule, or per tablet, or per total number of minitablets, e.g., used in a capsule.

### Methods of Use

The pharmaceutical agents, pharmaceutical compositions, and/or solid dosage forms described herein allow, e.g., for oral administration of a pharmaceutical agent contained therein.

The pharmaceutical agent can be combined with (for example, mixed into) a liquid (for example, a buffer, juice, or water), e.g., for oral administration of a pharmaceutical agent contained therein.

The pharmaceutical agents, pharmaceutical compositions, and/or solid dosage forms described herein can be used in the treatment and/or prevention of a cancer, inflammation, autoimmunity, a metabolic condition, or a dysbiosis.

Methods of using a pharmaceutical agent, pharmaceutical composition, and/or solid dosage form (e.g., for oral administration) (e.g., for pharmaceutical use), wherein the pharmaceutical agent comprises bacteria, are described herein.

The methods and administered pharmaceutical agents, pharmaceutical compositions, and/or solid dosage forms described herein allow, e.g., for oral administration of a pharmaceutical agent contained therein. The pharmaceutical agent, pharmaceutical composition, and/or solid dosage form can be administered to a subject is a fed or fasting state. The pharmaceutical agent, pharmaceutical composition, and/or solid dosage form can be administered, e.g., on an empty stomach (e.g., one hour before eating or two hours after eating). The pharmaceutical agent, pharmaceutical composition, and/or solid dosage form can be administered one hour before eating. The pharmaceutical agent, pharmaceutical composition, and/or solid dosage form can be administered two hours after eating.

A pharmaceutical agent, pharmaceutical composition, and/or solid dosage form for use in the treatment and/or prevention of a cancer, inflammation, autoimmunity, a metabolic condition, or a dysbiosis is provided herein.

Use of a pharmaceutical agent, pharmaceutical composition, and/or solid dosage form for the preparation of a medicament for the treatment and/or prevention of a cancer, inflammation, autoimmunity, a metabolic condition, or a dysbiosis is provided herein.

### Methods of Making Pharmaceutical Agents

In some aspects, the disclosure provides a method of preparing a pharmaceutical agent, wherein the pharmaceutical agent comprises bacteria (e.g., freeze dried bacteria). The pharmaceutical agent can be a powder, e.g., a freeze-dried powder.

The method can comprise combining bacteria (e.g., a pellet comprising bacteria) with a cryoprotectant solution, thereby preparing a formulated paste.

In some embodiments, the method further comprises freeze drying the formulated paste, to thereby prepare a freeze-dried product. In some embodiments, the freeze drying comprises primary drying. In some embodiments, the freeze drying comprises primary drying and secondary drying.

In some embodiments, the method further comprises milling the freeze-dried product, to thereby prepare a freeze-dried powder (e.g., powder, e.g., pharmaceutical agent).

In some embodiments, the cryoprotectant solution is mixed with the pellet in a ratio of about 0.2 to about 0.5 gram (g) cryoprotectant solution per gram of pellet; about 0.05 to about 0.25 gram (g) cryoprotectant solution per gram of pellet; about 0.06 to about 0.1 gram (g) cryoprotectant solution per gram of pellet; or about 0.15 to about 0.2 gram (g) cryoprotectant solution per gram of pellet. In some embodiments, the cryoprotectant solution is mixed with the pellet in a ratio of about 0.4 gram (g) cryoprotectant solution per gram of pellet. In some embodiments, the cryoprotectant solution is mixed with the pellet in a ratio of about 0.18 gram (g) cryoprotectant solution per gram of pellet. In some embodiments, the cryoprotectant solution is mixed with the pellet in a ratio of about 0.1 gram (g) (such as 0.08 g) cryoprotectant solution per gram of pellet.

In some embodiments, the cryoprotectant solution is mixed with the pellet at a ratio of 6.5% (volume/volume).

In some embodiments, the cryoprotectant solution comprises sucrose. In some embodiments, the cryoprotectant solution comprises dextran. In some embodiments, the cryoprotectant solution comprises sucrose and dextran. In some embodiments, the cryoprotectant solution comprises sucrose and dextran in equivalent amounts (e.g., on a percent weight by weight basis). In some embodiments, the cryoprotectant solution comprises sucrose, dextran, and L-cysteine HCl. In some embodiments, the cryoprotectant solution does not comprise L-cysteine HCl.

In some embodiments, the cryoprotectant solution comprises about 10% to about 30% (weight/weight) sucrose. In some embodiments, the cryoprotectant solution comprises about 15% to about 35% (weight/weight) sucrose. In some embodiments, the cryoprotectant solution comprises about 20% (weight/weight) sucrose.

In some embodiments, the cryoprotectant solution comprises about 10% to about 30% (weight/weight) dextran. In some embodiments, the cryoprotectant solution comprises about 15% to about 35% (weight/weight) dextran. In some embodiments, the cryoprotectant solution comprises about 20% (weight/weight) dextran.

In some embodiments, the cryoprotectant solution comprises about 10% to about 30% (weight/weight) sucrose and about 10% to about 30% (weight/weight) dextran. In some embodiments, the cryoprotectant solution comprises about 15% to about 35% (weight/weight) sucrose and about 15% to about 35% (weight/weight) dextran. In some embodiments, the cryoprotectant solution comprises about 20% (weight/weight) sucrose and about 20% (weight/weight) dextran.

In some embodiments, the cryoprotectant solution comprises about 40% to about 80% (weight/weight) water. In some embodiments, the cryoprotectant solution comprises about 50% to about 70% (weight/weight) water. In some embodiments, the cryoprotectant solution comprises about 55% to about 65% (weight/weight) water. In some embodiments, the cryoprotectant solution comprises about 60% (weight/weight) water.

In some embodiments, the cryoprotectant solution comprises about 0.05% to about 0.6% (weight/weight) L-cysteine HCl. In some embodiments, the cryoprotectant solution comprises about 0.1% to about 0.5% (weight/weight) L-cysteine HCl. In some embodiments, the cryoprotectant solution comprises about 0.15% to about 0.45% (weight/weight) L-cysteine HCl.

In some embodiments, the cryoprotectant solution comprises about 0.2% (weight/weight) L-cysteine HCl.

In some embodiments, the cryoprotectant solution comprises about 59.8% (weight/weight) water.

In some embodiments, the cryoprotectant solution comprises about 0.4% (weight/weight) L-cysteine HCl.

In some embodiments, the cryoprotectant solution comprises about 59.6% (weight/weight) water.

In certain embodiments, the cryoprotectant solution provided herein comprises: (i) about 20% (weight/weight) sucrose; (ii) about 20% (weight/weight) dextran; (iii) about 0.4% (weight/weight) L-cysteine HCl; and (iv) about 59.6% (weight/weight) water.

In certain embodiments, the cryoprotectant solution provided herein comprises: (i) about 20% (weight/weight) sucrose; (ii) about 20% (weight/weight) dextran; (iii) about 0.2% (weight/weight) L-cysteine HCl; and (iv) about 59.8% (weight/weight) water.

In certain embodiments, the cryoprotectant solution provided herein comprises: (i) about 20% (weight/weight) sucrose; (ii) about 20% (weight/weight) dextran; and (iii) about 60% (weight/weight) water.

In some embodiments, the cryoprotectant (e.g., dry composition not containing water) comprises about 40% to about 60% (weight/weight) sucrose. In some embodiments, the cryoprotectant comprises about 45% to about 55% (weight/weight) sucrose. In some embodiments, the cryoprotectant comprises about 50% (weight/weight) sucrose.

In some embodiments, the cryoprotectant comprises about 40% to about 60% (weight/weight) dextran. In some embodiments, the cryoprotectant comprises about 45% to about 55% (weight/weight) dextran. In some embodiments, the cryoprotectant comprises about 50% (weight/weight) dextran.

In some embodiments, the cryoprotectant comprises about 40% to about 60% (weight/weight) sucrose and 40% to about 60% (weight/weight) dextran. In some embodiments, the cryoprotectant comprises about 45% to about 55% (weight/weight) sucrose and about 45% to about 55% (weight/weight) dextran. In some embodiments, the cryoprotectant comprises about 50% (weight/weight) sucrose and about 50% (weight/weight) dextran.

In some embodiments, the cryoprotectant comprises about 0.25% to about 5% (weight/weight) L-cysteine HCl (e.g., a form of L-cysteine). In some embodiments, the cryoprotectant comprises about 0.5% to about 2.5% (weight/weight) L-cysteine HCl. In some embodiments, the cryoprotectant comprises about 0.75% to about 1.5% (weight/weight) L-cysteine HCl. In some embodiments, the cryoprotectant comprises about 1% (weight/weight) L-cysteine HCl.

In certain embodiments, the cryoprotectant provided herein comprises: (i) about 50% (weight/weight) sucrose; (ii) about 50% (weight/weight) dextran; and (iii) about 1% (weight/weight) L-cysteine HCl.

In certain embodiments, the cryoprotectant provided herein comprises: (i) about 50% (weight/weight) sucrose; and (ii) about 50% (weight/weight) dextran.

In some embodiments, the freeze-dried powder comprises about 6% to about 12% (weight/weight) sucrose. In some embodiments, the freeze-dried powder comprises about 8% to about 12% (weight/weight) sucrose. In some embodiments, the freeze-dried powder comprises about 11% (weight/weight) sucrose. In some embodiments, the freeze-dried powder comprises about 6% to about 12% (weight/weight) dextran. In some embodiments, the freeze-dried powder comprises about 8% to about 12% (weight/weight) dextran. In some embodiments, the freeze-dried powder comprises about 11% (weight/weight) dextran. In some embodiments, the freeze-dried powder comprises about 6% to about 12% (weight/weight) sucrose and about 6% to about 12% (weight/weight) dextran. In some embodiments, the freeze-dried powder comprises about 8% to about 12% (weight/weight) sucrose and about 8% to about 12% (weight/weight) dextran. In some embodiments, the freeze-dried powder comprises about 11% (weight/weight) sucrose and about 11% (weight/weight) dextran. In some embodiments, the freeze-dried powder comprises about 0.01% (weight/weight) L-cysteine HCl. In some embodiments, the pharmaceutical agent comprises about 0.1% to about 0.3% (weight/weight) L-cysteine HCl. In some embodiments, the pharmaceutical agent comprises about 0.15% to about 0.25% (weight/weight) L-cysteine HCl.

In some embodiments, the pharmaceutical agent comprises about 7% to about 21% (weight/weight) sucrose. In some embodiments, the pharmaceutical agent comprises about 10% to about 19% (weight/weight) sucrose. In some embodiments, the pharmaceutical agent comprises about 15% (weight/weight) sucrose. In some embodiments, the pharmaceutical agent comprises about 7% to about 21% (weight/weight) dextran. In some embodiments, the pharmaceutical agent comprises about 10% to about 19% (weight/weight) dextran. In some embodiments, the pharmaceutical agent comprises about 15% (weight/weight) dextran. In some embodiments, the pharmaceutical agent comprises about 7% to about 21% (weight/weight) sucrose and about 7% to about 21% (weight/weight) dextran. In some embodiments, the pharmaceutical agent comprises about 10% to about 19% (weight/weight) sucrose and about 10% to about 19% (weight/weight) dextran. In some embodiments, the pharmaceutical agent comprises about 15% (weight/weight) sucrose and about 15% (weight/weight) dextran. In some embodiments, the pharmaceutical agent comprises about 0.01% (weight/weight) L-cysteine HCl. In some embodiments, the pharmaceutical agent comprises about 0.1% to about 0.4% (weight/weight) L-cysteine HCl. In some embodiments, the pharmaceutical agent comprises about 0.15% to about 0.35% (weight/weight) L-cysteine HCl.

In certain embodiments, the freeze-dried powder comprises about 40% to about 70% (weight/weight) dried bacteria. In certain embodiments, the pharmaceutical agent comprises about 35% to about 70% (weight/weight) bacteria (e.g., freeze dried bacteria).

In certain embodiments, the freeze-dried powder comprises about 64% (weight/weight) dried bacteria.

In some embodiments, the cryoprotectant comprises a dried (dehydrated) maize glucose syrup (such as Glucidex), such as a dried maize glucose syrup with a dextrose equivalent (DE) of 21 (such as Glucidex 21). In some embodiments, the cryoprotectant comprises sucrose. In some embodiments, the cryoprotectant comprises dextrose (also referred to as glucose). In some embodiments, the cryoprotectant comprises monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and dextrose. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, and monosodium glutamate. In some embodiments, the cryoprotectant comprises dextrose and monosodium glutamate in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, monosodium glutamate, and L-cysteine HCl (e.g., a form of L-cysteine). In some embodiments, the cryoprotectant does not comprise L-cysteine HCl.

In some embodiments, the cryoprotectant (e.g., dry composition not containing water) comprises about 27% to about 47% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21).

In some embodiments, the cryoprotectant (e.g., dry composition not containing water) comprises about 27% to about 47% (weight/weight) sucrose. In some embodiments, the cryoprotectant comprises about 32% to about 42% (weight/weight) sucrose. In some embodiments, the cryoprotectant comprises about 37% (weight/weight) sucrose.

In some embodiments, the cryoprotectant comprises about 8% to about 18% (weight/weight) dextrose. In some embodiments, the cryoprotectant comprises about 11% to about 15% (weight/weight) dextrose. In some embodiments, the cryoprotectant comprises about 13% (weight/weight) dextrose.

In some embodiments, the cryoprotectant comprises about 8% to about 18% (weight/weight) monosodium glutamate. In some embodiments, the cryoprotectant comprises about 11% to about 15% (weight/weight) monosodium glutamate. In some embodiments, the cryoprotectant comprises about 13% (weight/weight) monosodium glutamate.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21) and about 27% to about 47% (weight/weight) sucrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21) and about 32% to about 42% (weight/weight) sucrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21) and about 37% (weight/weight) sucrose.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) dextrose. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) monosodium glutamate. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; about 8% to about 18% (weight/weight) dextrose; and about 8% to about 18% (weight/weight) monosodium glutamate.

In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) dextrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) monosodium glutamate. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; about 11% to about 15% (weight/weight) dextrose; and about 11% to about 15% (weight/weight) monosodium glutamate.

In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) dextrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) monosodium glutamate. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; about 13% (weight/weight) dextrose; and about 13% (weight/weight) monosodium glutamate.

In some embodiments, the cryoprotectant comprises about 0.05% to about 0.6% (weight/weight) L-cysteine HCl (e.g., a form of L-cysteine). In some embodiments, the cryoprotectant comprises about 0.1% to about 0.5% (weight/weight) L-cysteine HCl. In some embodiments, the cryoprotectant comprises about 0.1% to about 0.25% (weight/weight) L-cysteine HCl. In some embodiments, the cryoprotectant comprises about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; (iv) about 13% (weight/weight) monosodium glutamate; and (v) about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; and (iv) about 13% (weight/weight) monosodium glutamate.

In some embodiments, the freeze-dried powder comprises about 23% to about 30% (weight/weight) dried (dehydrated) maize glucose syrup (such as Glucidex), such as a dried maize glucose syrup with a dextrose equivalent (DE) of 21 (such as Glucidex 21). In some embodiments, the freeze-dried powder comprises about 24% to about 28% (weight/weight) dried (dehydrated) maize glucose syrup (such as Glucidex), such as a dried maize glucose syrup with a dextrose equivalent (DE) of 21 (such as Glucidex 21). In some embodiments, the freeze-dried powder comprises about 26% (weight/weight) dried (dehydrated) maize glucose syrup (such as Glucidex), such as a dried maize glucose syrup with a dextrose equivalent (DE) of 21 (such as Glucidex 21). In some embodiments, the freeze-dried powder comprises about 21% to about 29% (weight/weight) sucrose. In some embodiments, the freeze-dried powder comprises about 23% to about 27% (weight/weight) sucrose. In some embodiments, the freeze-dried powder comprises about 25% (weight/weight) sucrose. In some embodiments, the freeze-dried powder comprises about 6% to about 11% (weight/weight) dextrose. In some embodiments, the freeze-dried powder comprises about 7% to about 10% (weight/weight) dextrose. In some embodiments, the freeze-dried powder comprises about 9% (weight/weight) dextrose. In some embodiments, the freeze-dried powder comprises about 4% to about 10% (weight/weight) glutamate. In some embodiments, the freeze-dried powder comprises about 5% to about 9% (weight/weight) glutamate. In some embodiments, the freeze-dried powder comprises about 7% (weight/weight) glutamate.

In some embodiments, the freeze-dried powder comprises about 23% to about 30% (weight/weight) dried (dehydrated) maize glucose syrup (such as Glucidex), such as a dried maize glucose syrup with a dextrose equivalent (DE) of 21 (such as Glucidex 21) and about 21% to about 29% (weight/weight) sucrose. In some embodiments, the freeze-dried powder comprises about 24% to about 28% (weight/weight) dried (dehydrated) maize glucose syrup (such as Glucidex), such as a dried maize glucose syrup with a dextrose equivalent (DE) of 21 (such as Glucidex 21) and about 23% to about 27% (weight/weight) sucrose. In some embodiments, the freeze-dried powder comprises about 0.05% to about 0.6% (weight/weight) L-cysteine HCl. In some embodiments, the freeze-dried powder comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 6% to about 11% (weight/weight) dextrose. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 4% to about 10% (weight/weight) glutamate. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; about 6% to about 11% (weight/weight) dextrose; and about 4% to about 10% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); about 23% to about 27% (weight/weight) sucrose; and about 7% to about 10% (weight/weight) dextrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; and about 5% to about 9% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; about 7% to about 1% (weight/weight) dextrose; and about 5% to about 9% (weight/weight) glutamate. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 26% (weight/weight) dried (dehydrated) maize glucose syrup (such as Glucidex), such as a dried maize glucose syrup with a dextrose equivalent (DE) of 21 (such as Glucidex 21). In some embodiments, the pharmaceutical agent comprises about 21% to about 29% (weight/weight) sucrose. In some embodiments, the pharmaceutical agent comprises about 23% to about 27% (weight/weight) sucrose. In some embodiments, the pharmaceutical agent comprises about 25% (weight/weight) sucrose. In some embodiments, the pharmaceutical agent comprises about 6% to about 11% (weight/weight) dextrose. In some embodiments, the pharmaceutical agent comprises about 7% to about 10% (weight/weight) dextrose. In some embodiments, the pharmaceutical agent comprises about 9% (weight/weight) dextrose. In some embodiments, the pharmaceutical agent comprises about 4% to about 10% (weight/weight) glutamate. In some embodiments, the pharmaceutical agent comprises about 5% to about 9% (weight/weight) glutamate. In some embodiments, the pharmaceutical agent comprises about 7% (weight/weight) glutamate.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21) and about 21% to about 29% (weight/weight) sucrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21) and about 23% to about 27% (weight/weight) sucrose. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 6% to about 11% (weight/weight) dextrose. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; and about 4% to about 10% (weight/weight) glutamate. In some embodiments, the pharmaceutical agent comprises about 23% to about 30% (weight/weight) dried (dehydrated) maize glucose syrup (such as Glucidex), such as a dried maize glucose syrup with a dextrose equivalent (DE) of 21 (such as Glucidex 21); about 21% to about 29% (weight/weight) sucrose; about 6% to about 11% (weight/weight) dextrose; and about 4% to about 10% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); about 23% to about 27% (weight/weight) sucrose; and about 7% to about 10% (weight/weight) dextrose. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; and about 5% to about 9% (weight/weight) glutamate. In some embodiments, the pharmaceutical agent comprises about 24% to about 28% (weight/weight) dried (dehydrated) maize glucose syrup (such as Glucidex), such as a dried maize glucose syrup with a dextrose equivalent (DE) of 21 (such as Glucidex 21); 23% to about 27% (weight/weight) sucrose; about 7% to about 1% (weight/weight) dextrose; and about 5% to about 9% (weight/weight) glutamate. In some embodiments, the pharmaceutical agent comprises about 0.05% to about 0.6% (weight/weight) L-cysteine HCl. In some embodiments, the pharmaceutical agent comprises about 0.1% to about 0.5% (weight/weight) L-cysteine HCl.

In some embodiments, the cryoprotectant comprises a dried (dehydrated) maize glucose syrup (such as Glucidex), such as a dried maize glucose syrup with a dextrose equivalent (DE) of 21 (such as Glucidex 21). In some embodiments, the cryoprotectant comprises sucrose. In some embodiments, the cryoprotectant comprises dextrose (also referred to as glucose). In some embodiments, the cryoprotectant comprises monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose. In some embodiments, the cryoprotectant comprises dried (dehydrated) maize glucose syrup (such as Glucidex), such as a dried maize glucose syrup with a dextrose equivalent (DE) of 21 (such as Glucidex 21) and sucrose in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and dextrose. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, and monosodium glutamate. In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, and monosodium glutamate. In some embodiments, the cryoprotectant comprises dextrose and monosodium glutamate in equivalent amounts (e.g., on a percent weight by weight basis). In some embodiments, the cryoprotectant comprises dried (dehydrated) maize glucose syrup (such as Glucidex), such as a dried maize glucose syrup with a dextrose equivalent (DE) of 21 (such as Glucidex 21), sucrose, dextrose, monosodium glutamate, and L-cysteine HCl (e.g., a form of L-cysteine). In some embodiments, the cryoprotectant does not comprise L-cysteine HCl.

In some embodiments, the cryoprotectant (e.g., dry composition not containing water) comprises about 27% to about 47% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21).

In some embodiments, the cryoprotectant (e.g., dry composition not containing water) comprises about 27% to about 47% (weight/weight) sucrose. In some embodiments, the cryoprotectant comprises about 32% to about 42% (weight/weight) sucrose. In some embodiments, the cryoprotectant comprises about 37% (weight/weight) sucrose.

In some embodiments, the cryoprotectant comprises about 8% to about 18% (weight/weight) dextrose. In some embodiments, the cryoprotectant comprises about 11% to about 15% (weight/weight) dextrose. In some embodiments, the cryoprotectant comprises about 13% (weight/weight) dextrose.

In some embodiments, the cryoprotectant comprises about 8% to about 18% (weight/weight) monosodium glutamate. In some embodiments, the cryoprotectant comprises about 11% to about 15% (weight/weight) monosodium glutamate. In some embodiments, the cryoprotectant comprises about 13% (weight/weight) monosodium glutamate.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21) and about 27% to about 47% (weight/weight) sucrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21) and about 32% to about 42% (weight/weight) sucrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21) and about 37% (weight/weight) sucrose.

In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) dextrose. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) monosodium glutamate. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; about 8% to about 18% (weight/weight) dextrose; and about 8% to about 18% (weight/weight) monosodium glutamate.

In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) dextrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) monosodium glutamate. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; about 11% to about 15% (weight/weight) dextrose; and about 11% to about 15% (weight/weight) monosodium glutamate.

In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) dextrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) monosodium glutamate. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; about 13% (weight/weight) dextrose; and about 13% (weight/weight) monosodium glutamate.

In some embodiments, the cryoprotectant comprises about 0.05% to about 0.6% (weight/weight) L-cysteine HCl (e.g., a form of L-cysteine). In some embodiments, the cryoprotectant comprises about 0.1% to about 0.5% (weight/weight) L-cysteine HCl. In some embodiments, the cryoprotectant comprises about 0.1% to about 0.25% (weight/weight) L-cysteine HCl. In some embodiments, the cryoprotectant comprises about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; (iv) about 13% (weight/weight) monosodium glutamate; and (v) about 0.2% (weight/weight) L-cysteine HCl.

In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; and (iv) about 13% (weight/weight) monosodium glutamate.

In certain embodiments, the freeze-dried powder comprises about 15% to about 35% (weight/weight) bacteria (e.g., freeze dried bacteria). In certain embodiments, the freeze-dried powder comprises about 18% to about 30% (weight/weight) bacteria (e.g., freeze dried bacteria). In certain embodiments, the freeze-dried powder comprises about 25% (weight/weight) bacteria (e.g., freeze dried bacteria).

In certain embodiments, the freeze-dried powder comprises Prevotella bacteria.

In certain embodiments, the freeze-dried powder comprises Veillonella bacteria.

As one example of a method of preparing a pharmaceutical agent, the method can include one or more of the following steps: centrifuging (e.g., using continuous centrifugation) a fermentation broth that contains bacteria to prepare a pellet that comprises bacteria; combining a cryoprotectant solution with the pellet to prepare a formulated paste; and freeze drying the formulated paste to prepare a freeze-dried product. The freeze drying can include primary drying (e.g., at −5° C.). Freeze drying can include secondary drying (e.g., at 25° C.). The total drying time can be, e.g., at least 48 hours. The freeze-dried product can be milling (e.g., using a milling machine) to prepare a freeze-dried powder, e.g., pharmaceutical agent. The freeze-dried powder can optionally be gamma irradiated.

In some embodiments, the method can further comprise combining the freeze-dried powder with one or more excipients to thereby prepare a pharmaceutical composition. The pharmaceutical composition can be used in methods as described herein. The pharmaceutical composition can be prepared as a solid dosage form (e.g., capsule, tablet, and/or mini-tablet) as described herein. The solid dosage form can be coated, e.g., enterically coated, e.g., as described herein.

In some embodiments, the disclosure provides a pharmaceutical agent prepared by a method described herein.

### Additional Aspects of the Solid Dosage Forms

The solid dosage forms, e.g., as described herein, comprising a pharmaceutical agent (e.g., a therapeutically effective amount thereof), wherein the pharmaceutical agent comprises bacteria, and wherein the solid dosage form further comprises excipients, can provide a therapeutically effective amount of the pharmaceutical agent to a subject, e.g., a human.

The solid dosage forms, e.g., as described herein, comprising a pharmaceutical agent (e.g., a therapeutically effective amount thereof), wherein the pharmaceutical agent comprises bacteria, and wherein the solid dosage form further comprises excipients, can provide a non-natural amount of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria, and wherein the solid dosage form further comprises excipients, can provide an unnatural quantity of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria, and wherein the solid dosage form further comprises excipients, can bring about one or more changes to a subject, e.g., human, e.g., to treat or prevent a disease or a health disorder.

The solid dosage forms, e.g., as described herein, comprising a pharmaceutical agent (e.g., a therapeutically effective amount thereof), wherein the pharmaceutical agent comprises bacteria, and wherein the solid dosage form further comprises excipients, has potential for significant utility, e.g., to affect a subject, e.g., a human, e.g., to treat or prevent a disease or a health disorder.

### Additional Therapeutic Agents

In certain aspects, the methods provided herein include the administration to a subject of a pharmaceutical agent, pharmaceutical composition, and/or solid dosage form described herein either alone or in combination with an additional therapeutic agent. In some embodiments, the additional therapeutic agent is an immunosuppressant, an anti-inflammatory agent, a steroid, and/or a cancer therapeutic.

In some embodiments, the pharmaceutical agent, pharmaceutical composition, and/or solid dosage form is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the pharmaceutical agent, pharmaceutical composition, and/or solid dosage form is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical agent, pharmaceutical composition, and/or solid dosage form and the additional therapeutic agent are administered to the subject simultaneously or nearly simultaneously (e.g., administrations occur within an hour of each other).

In some embodiments, an antibiotic is administered to the subject before the pharmaceutical agent, pharmaceutical composition, and/or solid dosage form is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after the pharmaceutical agent, pharmaceutical composition, and/or solid dosage form is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical agent, pharmaceutical composition, and/or solid dosage form and the antibiotic are administered to the subject simultaneously or nearly simultaneously (e.g., administrations occur within an hour of each other).

In some embodiments, the additional therapeutic agent is a cancer therapeutic. In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject's immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. The immunotherapy agent may be administered via injection (e.g., intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-Ai110, TSR-042, RG-7446, BMS-936559, MEDI-4736, MSB-0020718C, AUR-012 and STI-A1010.

In some embodiments, the methods provided herein include the administration of a pharmaceutical agent, pharmaceutical composition, and/or solid dosage form described herein in combination with one or more additional therapeutic agents. In some embodiments, the methods disclosed herein include the administration of two immunotherapy agents (e.g., immune checkpoint inhibitor). For example, the methods provided herein include the administration of a pharmaceutical agent, pharmaceutical composition, and/or solid dosage form described herein in combination with a PD-1 inhibitor (such as pemrolizumab or nivolumab or pidilizumab) or a CLTA-4 inhibitor (such as ipilimumab) or a PD-L1 inhibitor.

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPPIR3B, PRAME, PRDX5, PSA, PSMA, PTPRK, RAB38/NY-MEL-1, RAGE-1, RBAF600, RGS5, RhoC, RNF43, RU2AS, SAGE, secemin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WTi, XAGE-1b/GAGED2a. In some embodiments, the antigen is a neo-antigen.

In some embodiments, the immunotherapy agent is a cancer vaccine and/or a component of a cancer vaccine (e.g., an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (e.g., DNA or RNA, such as mRNA) that encodes an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPPIR3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen. In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A, cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“IFN-beta”) Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”), Interleukin-18 (“IL-18”), Interleukin-21 (“IL-21”), Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 RI, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFRlAdiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member IOA (“TRAIL R1”), Transferrin (“TRF”), WIF-IACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κB (“RANK”).

In some embodiments, the cancer therapeutic is an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

Exemplary anti-cancer compounds that modify the function of proteins that regulate gene expression and other cellular functions (e.g., HDAC inhibitors, retinoid receptor ligants) are Vorinostat (Zolinza®), Bexarotene (Targretin®) and Romidepsin (Istodax®), Alitretinoin (Panretin®), and Tretinoin (Vesanoid®).

Exemplary anti-cancer compounds that induce apoptosis (e.g., proteasome inhibitors, antifolates) are Bortezomib (Velcade®), Carfilzomib (Kyprolis™), and Pralatrexate (Folotyn®).

Exemplary anti-cancer compounds that increase anti-tumor immune response (e.g., anti CD20, anti CD52; anti-cytotoxic T-lymphocyte-associated antigen-4) are Rituximab (Rituxan®), Alemtuzumab (Campath®), Ofatumumab (Arzerra®), and Ipilimumab (Yervoy™).

Exemplary anti-cancer compounds that deliver toxic agents to cancer cells (e.g., anti-CD20-radionuclide fusions; IL-2-diphtheria toxin fusions; anti-CD30-monomethylauristatin E (MMAE)-fusions) are Tositumomab and 131I-tositumomab (Bexxar®) and Ibritumomab tiuxetan (Zevalin®), Denileukin diftitox (Ontak®), and Brentuximab vedotin (Adcetris®).

Other exemplary anti-cancer compounds are small molecule inhibitors and conjugates thereof of, e.g., Janus kinase, ALK, Bcl-2, PARP, PI3K, VEGF receptor, Braf, MEK, CDK, and HSP90.

Exemplary platinum-based anti-cancer compounds include, for example, cisplatin, carboplatin, oxaliplatin, satraplatin, picoplatin, Nedaplatin, Triplatin, and Lipoplatin. Other metal-based drugs suitable for treatment include, but are not limited to ruthenium-based compounds, ferrocene derivatives, titanium-based compounds, and gallium-based compounds.

In some embodiments, the cancer therapeutic is a radioactive moiety that comprises a radionuclide. Exemplary radionuclides include, but are not limited to Cr-51, Cs-131, Ce-134, Se-75, Ru-97, I-125, Eu-149, Os-189m, Sb-119, I-123, Ho-161, Sb-117, Ce-139, In-111, Rh-103m, Ga-67, Tl-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sm-153, Gd-159, Tm-173, Pr-143, Au-198, Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, the additional therapeutic is an antibiotic. For example, if the presence of a disease-associated bacteria and/or a disease-associated microbiome profile is detected, antibiotics can be administered, e.g., to eliminate the disease-associated bacteria from the subject. In some embodiments, the cancer therapeutic is an antibiotic. For example, if the presence of a cancer-associated bacteria and/or a cancer-associated microbiome profile is detected according to the methods provided herein, antibiotics can be administered to eliminate the cancer-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a disease (such as cancer) niche may be used to target disease-associated microbes, including disease-associated bacteria in that niche. In other embodiments, antibiotics are administered after the solid dosage form. In some embodiments, antibiotics are administered before the solid dosage form.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin P1, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal antiinflammatory drug (NSAID), or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lomoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MablIera®), ABATACEPT (Orencia®), TOCILIZUMAB (RoActemra/Actemra®), integrin antagonists (TYSABRI® (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta®/LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D—ergocalciferol, Milnacipran, Paclitaxel, rosig tazone, Tacrolimus (Prograf®), RADOOl, rapamune, rapamycin, fostamatinib, Fentanyl, XOMA 052, Fostamatinib disodium, rosightazone, Curcumin (Longvida™), Rosuvastatin, Maraviroc, ramipnl, Milnacipran, Cobiprostone, somatropin, tgAAC94 gene therapy vector, MK0359, GW856553, esomeprazole, everolimus, trastuzumab, JAKI and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (Tysarbri® (natalizumab)), VGEF antagnosits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

In some embodiments, the additional therapeutic agent is an RNA molecule, such as a double stranded RNA.

In some embodiments, the additional therapeutic agent is an anti-sense oligonucleotide.

### Administration

In certain aspects, provided herein is a method of delivering a pharmaceutical agent, pharmaceutical composition, and/or solid dosage form described herein to a subject. In some embodiments of the methods provided herein, the pharmaceutical agent, pharmaceutical composition, and/or solid dosage form that comprises bacteria is administered in conjunction with the administration of an additional therapeutic agent. In some embodiments, in the pharmaceutical agent, pharmaceutical composition, and/or solid dosage form, a pharmaceutical agent is co-formulated with the additional therapeutic agent. In some embodiments, the pharmaceutical agent, pharmaceutical composition, and/or solid dosage form is co-administered with the additional therapeutic agent. In some embodiments, the additional therapeutic agent is administered to the subject before administration of the pharmaceutical agent, pharmaceutical composition, and/or solid dosage form (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the pharmaceutical agent, pharmaceutical composition, and/or solid dosage form (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the pharmaceutical agent, pharmaceutical composition, and/or solid dosage form and the additional therapeutic agent. In some embodiments, different modes of delivery are used to administer the pharmaceutical agent, pharmaceutical composition, and/or solid dosage form and the additional therapeutic agent. For example, in some embodiments the pharmaceutical agent, pharmaceutical composition, and/or solid dosage form is administered orally while the additional therapeutic agent is administered via injection (e.g., an intravenous, intramuscular and/or intratumoral injection).

In certain embodiments, the pharmaceutical agent, pharmaceutical composition, and/or solid dosage form described herein can be administered in conjunction with any other conventional anti-cancer treatment, such as, for example, radiation therapy and surgical resection of the tumor. These treatments may be applied as necessary and/or as indicated and may occur before, concurrent with or after administration of the pharmaceutical agent, pharmaceutical composition, and/or solid dosage form described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, tumor size, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical agent (e.g., in a solid dosage form) described herein may be appropriately set or adjusted in accordance with the dosage form, the route of administration, the degree or stage of a target disease, and the like.

In some embodiments, the dose administered to a subject is sufficient to prevent disease (e.g., autoimmune disease, inflammatory disease, metabolic disease, dysbiosis, or cancer), delay its onset, or slow or stop its progression, or relieve one or more symptoms of the disease. One skilled in the art will recognize that dosage will depend upon a variety of factors including the strength of the particular agent (e.g., pharmaceutical agent) employed, as well as the age, species, condition, and body weight of the subject. The size of the dose will also be determined by the route, timing, and frequency of administration as well as the existence, nature, and extent of any adverse side-effects that might accompany the administration of a particular pharmaceutical agent and the desired physiological effect.

Suitable doses and dosage regimens can be determined by conventional range-finding techniques known to those of ordinary skill in the art. Generally, treatment is initiated with smaller dosages, which are no more than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the pharmaceutical agents, pharmaceutical compositions, and/or solid dosage forms used in accordance with the invention vary depending on the active agent, the age, weight, and clinical condition of the recipient patient, and the experience and judgment of the clinician or practitioner administering the therapy, among other factors affecting the selected dosage. For example, for cancer treatment, the dose should be sufficient to result in slowing, and preferably regressing, the growth of a tumor and most preferably causing complete regression of the cancer, or reduction in the size or number of metastases As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a pharmaceutical agent, pharmaceutical composition, and/or solid dosage form, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be no more than the time period for a subject to mount an immune response, such as no more than about one week, no more than about ten days, no more than about two weeks, or no more than about a month.

In some embodiments, the delivery of an additional therapeutic agent in combination with the pharmaceutical agent, pharmaceutical composition, and/or solid dosage form described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylasix, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and pharmaceutical agents, pharmaceutical compositions, and/or solid dosage forms described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis. In some embodiments, the disease or disorder is atopic dermatitis.

The methods and pharmaceutical agents, pharmaceutical compositions, and/or solid dosage forms described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring such a disease or disorder.

The pharmaceutical agents, pharmaceutical compositions, and/or solid dosage forms described herein can be used, for example, for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of immune cells.

In some embodiments, the methods and pharmaceutical agents, pharmaceutical compositions, and/or solid dosage forms provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to an immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and solid dosage forms described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and solid dosage forms described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and solid dosage forms described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and pharmaceutical agents, pharmaceutical compositions, and/or solid dosage forms described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, Goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and pharmaceutical agents, pharmaceutical compositions, and/or solid dosage forms described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and pharmaceutical agents, pharmaceutical compositions, and/or solid dosage forms include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, comea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Metabolic Disorders

In some embodiments, the methods and pharmaceutical agents, pharmaceutical compositions, and/or solid dosage forms described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), nonalcoholic steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of nonalcoholic fatty liver disease (NAFLD) and nonalcoholic steatohepatitis (NASH).

The methods and pharmaceutical agents, pharmaceutical compositions, and/or solid dosage forms described herein can be used to treat any subject in need thereof. As used herein, a “subject in need thereof” includes any subject that has a metabolic disease or disorder, as well as any subject with an increased likelihood of acquiring such a disease or disorder.

The pharmaceutical agents, pharmaceutical compositions, and/or solid dosage forms described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), nonalcoholic steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Cancer

In some embodiments, the methods and pharmaceutical agents, pharmaceutical compositions, and/or solid dosage forms described herein relate to the treatment of cancer. In some embodiments, any cancer can be treated using the methods described herein. Examples of cancers that may treated by methods and pharmaceutical agents, pharmaceutical compositions, and/or solid dosage forms described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises breast cancer (e.g., triple negative breast cancer).

In some embodiments, the cancer comprises colorectal cancer (e.g., microsatellite stable (MSS) colorectal cancer).

In some embodiments, the cancer comprises renal cell carcinoma.

In some embodiments, the cancer comprises lung cancer (e.g., non-small cell lung cancer).

In some embodiments, the cancer comprises bladder cancer.

In some embodiments, the cancer comprises gastroesophageal cancer.

In some embodiments, the methods and pharmaceutical agents, pharmaceutical compositions, and/or solid dosage forms provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and pharmaceutical agents, pharmaceutical compositions, and/or solid dosage forms provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and pharmaceutical agents, pharmaceutical compositions, and/or solid dosage forms provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and pharmaceutical agents, pharmaceutical compositions, and/or solid dosage forms described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and pharmaceutical agents, pharmaceutical compositions, and/or solid dosage forms described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypemephroma, hypemephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer's lip), cutaneous homs, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Other Diseases and Disorders

In some embodiments, the methods and pharmaceutical agents, pharmaceutical compositions, and/or solid dosage forms described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors, Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts, Liver Cancer, Newborn Jaundice, Primary Biliary Cholangitis (PBC), Primary Sclerosing Cholangitis (PSC), Reye Syndrome, Type I Glycogen Storage Disease, and Wilson Disease.

The methods and pharmaceutical agents, pharmaceutical compositions, and/or solid dosage forms described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

In recent years, it has become increasingly clear that the gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25. 2017; Weiss and Thierry, Mechanisms and consequences of intestinal dysbiosis. Cellular and Molecular Life Sciences. (2017) 74(16):2959-2977. Zurich Open Repository and Archive, doi: https://doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. October 2017. Vol. 8. Issue 5. mBio 8:e01492-17. https://doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018); Srinivasan et al., TEER measurement techniques for in vitro barrier model systems. J. Lab. Autom. 20:107-126 (2015). A gastrointestinal dysbiosis can have physiological and immune effects within and outside the gastrointestinal tract.

The presence of a dysbiosis has been associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn's disease), neuroinflammatory diseases (e.g., multiple sclerosis), transplant disorders (e.g., graft-versus-host disease), fatty liver disease, type I diabetes, rheumatoid arthritis, Sjögren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med. 375:2369-79 (2016), Carding et al., Dysbiosis of the gut microbiota in disease. Microb. Ecol. Health Dis. (2015); 26: 10: 3402/mehd.v26.2619; Levy et al, Dysbiosis and the Immune System, Nature Reviews Immunology 17:219 (April 2017).

Exemplary pharmaceutical agents, pharmaceutical compositions, and/or solid dosage forms disclosed herein can treat a dysbiosis and its effects by modifying the immune activity present at the site of dysbiosis. As described herein, such compositions can modify a dysbiosis via effects on host immune cells, resulting in, e.g., an increase in secretion of anti-inflammatory cytokines and/or a decrease in secretion of pro-inflammatory cytokines, reducing inflammation in the subject recipient or via changes in metabolite production.

Exemplary pharmaceutical agents, pharmaceutical compositions, and/or solid dosage forms disclosed herein that are useful for treatment of disorders associated with a dysbiosis contain one or more types of immunomodulatory bacteria (e.g., anti-inflammatory bacteria). Such compositions are capable of affecting the recipient host's immune function, in the gastrointestinal tract, and/or a systemic effect at distal sites outside the subject's gastrointestinal tract.

Exemplary pharmaceutical agents, pharmaceutical compositions, and/or solid dosage forms disclosed herein that are useful for treatment of disorders associated with a dysbiosis contain a population of immunomodulatory bacteria of a single bacterial species (e.g., a single strain) (e.g., anti-inflammatory bacteria). Such compositions are capable of affecting the recipient host's immune function, in the gastrointestinal tract, and/or a systemic effect at distal sites outside the subject's gastrointestinal tract.

In one embodiment, pharmaceutical agents, pharmaceutical compositions, and/or solid dosage forms containing an isolated population of immunomodulatory bacteria (e.g., anti-inflammatory bacterial cells) are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, pharmaceutical agents, pharmaceutical compositions, and/or solid dosage forms of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the pharmaceutical agents, pharmaceutical compositions, and/or solid dosage forms can treat a gastrointestinal dysbiosis and one or more of its effects by modulating the recipient immune response via cellular and cytokine modulation to reduce gut permeability by increasing the integrity of the intestinal epithelial barrier.

In another embodiment, the pharmaceutical agents, pharmaceutical compositions, and/or solid dosage forms can treat a distal dysbiosis and one or more of its effects by modulating the recipient immune response at the site of dysbiosis via modulation of host immune cells.

Other exemplary pharmaceutical agents, pharmaceutical compositions, and/or solid dosage forms are useful for treatment of disorders associated with a dysbiosis, which compositions contain one or more types of bacteria capable of altering the relative proportions of host immune cell subpopulations, e.g., subpopulations of T cells, immune lymphoid cells, dendritic cells, NK cells and other immune cells, or the function thereof, in the recipient.

Other exemplary pharmaceutical agents, pharmaceutical compositions, and/or solid dosage forms are useful for treatment of disorders associated with a dysbiosis, which compositions contain a population of immunomodulatory bacteria of a single bacterial species e.g., a single strain) capable of altering the relative proportions of immune cell subpopulations, e.g., T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a pharmaceutical agent, pharmaceutical composition, and/or solid dosage form which alters the microbiome population existing at the site of the dysbiosis. The pharmaceutical agent, pharmaceutical composition, and/or solid dosage form can contain one or more types of immunomodulatory bacteria or a population of immunomodulatory bacteria of a single bacterial species (e.g., a single strain).

In one embodiment, the invention provides methods of treating a distal dysbiosis and one or more of its effects by orally administering to a subject in need thereof a pharmaceutical agent, pharmaceutical composition, and/or solid dosage form which alters the subject's immune response outside the gastrointestinal tract. The pharmaceutical agent, pharmaceutical composition, and/or solid dosage form can contain one or more types of immunomodulatory bacteria or a population of immunomodulatory bacteria of a single bacterial species (e.g., a single strain).

In exemplary embodiments, pharmaceutical agents, pharmaceutical compositions, and/or solid dosage forms useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical agents, pharmaceutical compositions, and/or solid dosage forms useful for treatment of disorders associated with a dysbiosis that decrease (e.g., inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising bacteria in an amount sufficient to alter the microbiome at a site of the dysbiosis, such that the disorder associated with the dysbiosis is treated.

In another embodiment, a therapeutic composition of the instant invention in the form of a probiotic or medical food may be used to prevent or delay the onset of a dysbiosis in a subject at risk for developing a dysbiosis.

### Methods of Making Enhanced Bacteria

In certain aspects, provided herein are methods of making engineered bacteria for the production of the bacteria described herein. In some embodiments, the engineered bacteria are modified to enhance certain desirable properties. For example, in some embodiments, the engineered bacteria are modified to enhance the immunomodulatory and/or therapeutic effect of the bacteria (e.g., either alone or in combination with another therapeutic agent), to reduce toxicity and/or to improve bacterial manufacturing (e.g., higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof.

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (e.g., by exposure to chemical mutagens and/or UV radiation) or exposing them to a therapeutic agent (e.g., antibiotic) followed by an assay to detect bacteria having the desired phenotype (e.g., an in vivo assay, an ex vivo assay, or an in vitro assay).

### Gamma-Irradiation: Sample Protocol:

Powders are gamma-irradiated at 17.5 kGy radiation unit at ambient temperature. Frozen biomasses are gamma-irradiated at 25 kGy radiation unit in the presence of dry ice.

### Frozen Biomass Preparation: Sample Protocol

After a desired level of bacterial culture growth is achieved, centrifuge cultures, discard the supernatant, leaving the pellet as dry as possible. Vortex the pellet to loosen the biomass. Resuspend pellet in desired cryoprotectant solution, transfer to cryogenic tube and snap freeze in liquid nitrogen. Store in −80 degree c. freezer.

### Powder Preparation: Sample Protocol

After desired level of bacterial culture growth is achieved, centrifuge cultures, discard the supernatant, leaving the pellet as dry as possible. Resuspend pellet in desired cryoprotectant solution to create a formulated paste. The cryoprotectant solution may contain, e.g., maltodextrin, sodium ascorbate, sodium glutamate, and/or calcium chloride or e.g., sucrose, dextran, and L-cysteine HCl. Load the formulated paste onto stainless steel trays and load into a freeze drier (e.g., operating in automated mode with defined cycle parameters) to prepare a freeze dried product. The freeze dried product is fed into a milling machine and the resulting powder is collected.

Powders can be stored (e.g., in vacuum sealed bags) at 2-8 degrees C. (e.g., at 4 degrees C.), e.g., in a desiccator.

### EXEMPLAR EMBODIMENTS

Embodiment 1. A pharmaceutical agent (e.g., powder) comprising bacteria (e.g., freeze dried bacteria), wherein the bacteria in the pharmaceutical agent are present at a total cell count (TCC) of at least 1×10¹¹ cells/gram of the pharmaceutical agent (e.g., the bacteria are present in the pharmaceutical agent at a total cell count (TCC) of at least 3.3×10¹¹ cells/gram, at least 5×10¹¹ cells/gram, at least 7×10¹¹ cells/gram, at least 1×10¹² cells/gram, at least 2×10¹² cells/gram, between about 1×10¹¹ cells/gram to about 2.5×10¹² cells/gram, between about 3.3×10¹¹ cells/gram to about 2.5×10¹² cells/gram, between about 5×10¹¹ cells/gram to about 2.5×10¹² cells/gram, between about 7×10¹¹ cells/gram to about 2.4×10¹² cells/gram, about 2×10¹² cells/gram, between about 1×10¹² cells/gram to about 2×10¹² cells/gram, between about 1.3×10¹² cells/gram to about 2.4×10¹² cells/gram, about 2.5×10¹² cells/gram, about 1.2×10¹² cells/gram, about 1.5×10¹² cells/gram, or about 6×10¹¹ cells/gram of the pharmaceutical agent).

Embodiment 2. The pharmaceutical agent of embodiment 1, wherein the bacteria are present in the pharmaceutical agent at a total cell count (TCC) of between about 1×10¹¹ cells/gram to about 2.5×10¹² cells/gram of the pharmaceutical agent.

Embodiment 3. A pharmaceutical agent (e.g., powder) comprising bacteria and a cryoprotectant.

Embodiment 4. The pharmaceutical agent of embodiment 3, wherein the cryoprotectant comprises sucrose, dextran, or a combination thereof, optionally the cryoprotectant comprises sucrose and dextran in equivalent amounts.

Embodiment 5. The pharmaceutical agent of embodiment 3, wherein the cryoprotectant comprises sucrose, dextran, and L-cysteine HCl.

Embodiment 6. The pharmaceutical agent of embodiment 3, wherein the cryoprotectant does not comprise L-cysteine HCl.

Embodiment 7. The pharmaceutical agent of embodiment 3, wherein the pharmaceutical agent comprises about 6% to about 12% or about 7% to about 21% (weight/weight) sucrose; about 6% to about 12% or about 7% to about 21% (weight/weight) dextran; or about 6% to about 12% (weight/weight) sucrose and about 6% to about 12% (weight/weight) dextran, or about 7% to about 21% (weight/weight) sucrose and about 7% to about 21% (weight/weight) dextran (e.g., wherein the pharmaceutical agent comprises about 8% to about 12% or about 10% to about 19% (weight/weight) sucrose, the pharmaceutical agent comprises about 11% (weight/weight) sucrose, the pharmaceutical agent comprises about 6% to about 12% or about 7% to about 21% (weight/weight) dextran, the pharmaceutical agent comprises about 8% to about 12% or about 10% to about 19% (weight/weight) dextran, the pharmaceutical agent comprises about 11% (weight/weight) dextran, the pharmaceutical agent comprises about 6% to about 12% (weight/weight) sucrose and about 6% to about 12% (weight/weight) dextran, or about 7% to about 21% (weight/weight) sucrose and about 7% to about 21% (weight/weight) dextran, the pharmaceutical agent comprises about 8% to about 12% (weight/weight) sucrose and about 8% to about 12% (weight/weight) dextran, or about 10% to about 19% (weight/weight) sucrose and about 10% to about 19% (weight/weight) dextran, or the pharmaceutical agent comprises about 11% (weight/weight) sucrose and about 11% (weight/weight) dextran).

Embodiment 8. The pharmaceutical agent of embodiment 3, wherein the pharmaceutical agent comprises about 0.10% to about 0.25% or about 0.15% to about 0.35% (weight/weight) L-cysteine HCl.

Embodiment 9. A pharmaceutical composition that comprises a pharmaceutical agent (e.g., powder) of any one of embodiments 1 to 10 and one or more excipients.

Embodiment 10. A method comprising combining bacteria (e.g., a pellet comprising bacteria) with a cryoprotectant, optionally the cryoprotectant is a cryoprotectant solution, thereby preparing a formulated paste.

Embodiment 11. The method of embodiment 10, further comprising: (a) freeze drying the formulated paste, to thereby prepare a freeze-dried product; (b) milling the freeze-dried product, to thereby prepare a freeze-dried powder; and/or (c) combining the freeze-dried powder with one or more excipients to thereby prepare a pharmaceutical composition.

Embodiment 12. The method of embodiment 10 or 11, wherein the cryoprotectant is mixed with the pellet in a ratio of 0.1 to 0.25 gram (g) cryoprotectant per gram of pellet (e.g., a ratio of 0.15 to 0.2 gram (g) cryoprotectant solution per gram of pellet, a ratio of about 0.18 gram (g) cryoprotectant solution per gram of pellet); or a ratio of 4% to 10% (volume/volume) (e.g., 5% to 8% (volume/volume), a ratio of about 6.5% (volume/volume)).

Embodiment 13. The method of any one of embodiments 10 to 12, wherein the cryoprotectant comprises sucrose, dextran, or a combination thereof, optionally the cryoprotectant comprises sucrose and dextran in equivalent amounts.

Embodiment 14. The method of any one of embodiments 10 to 12, wherein the cryoprotectant comprises sucrose, dextran, and L-cysteine HCl.

Embodiment 15. The method of any one of embodiments 10 to 12, wherein the cryoprotectant does not comprise L-cysteine HCl.

Embodiment 16. The method of any one of embodiments 10 to 12, wherein the cryoprotectant comprises about 10% to about 30% (weight/weight) sucrose (e.g., about 15% to about 35% (weight/weight), about 20% (weight/weight)); about 10% to about 30% (weight/weight) dextran (e.g., about 15% to about 35% (weight/weight), about 20% (weight/weight)); or about 10% to about 30% (weight/weight) sucrose and about 10% to about 30% (weight/weight) dextran (e.g., about 15% to about 35% (weight/weight) sucrose and about 15% to about 35% (weight/weight) dextran, about 20% (weight/weight) sucrose and about 20% (weight/weight) dextran).

Embodiment 17. The method of any one of embodiments 10 to 12, wherein the cryoprotectant comprises about 40% to about 80% (weight/weight) water (e.g., about 50% to about 70% (weight/weight) water, about 55% to about 65% (weight/weight) water, about 60% (weight/weight) water), about 59.8% (weight/weight) water, about 59.6% (weight/weight) water).

Embodiment 18. The method of any one of embodiments 10 to 12, wherein the cryoprotectant comprises about 0.05% to about 0.6% (weight/weight) L-cysteine HC (e.g., about 0.1% to about 0.5% (weight/weight) L-cysteine HCl, about 0.15% to about 0.45% (weight/weight) L-cysteine HCl, about 0.2% (weight/weight) L-cysteine HCl, about 0.4% (weight/weight) L-cysteine HCl, about 0.1% (weight/weight) L-cysteine HCl).

Embodiment 19. The method of any one of embodiments 10 to 12, wherein the cryoprotectant comprises about 0.25% to about 5% (weight/weight) L-cysteine HCl. In some embodiments, the cryoprotectant solution provided herein comprises: (i) about 20% (weight/weight) sucrose; (ii) about 20% (weight/weight) dextran; (iii) about 0.4% (weight/weight) L-cysteine HCl; and (iv) about 59.6% (weight/weight) water. In some embodiments, the cryoprotectant solution provided herein comprises: (i) about 20% (weight/weight) sucrose; (ii) about 20% (weight/weight) dextran; (iii) about 0.2% (weight/weight) L-cysteine HCl; and (iv) about 59.8% (weight/weight) water. In some embodiments, the cryoprotectant solution provided herein comprises: (i) about 20% (weight/weight) sucrose; (ii) about 20% (weight/weight) dextran; and (iii) about 60% (weight/weight) water. In some embodiments, the cryoprotectant of the cryoprotectant solution comprises about 40% to about 60% (weight/weight) sucrose. In some embodiments, the cryoprotectant of the cryoprotectant solution comprises about 45% to about 55% (weight/weight) sucrose. In some embodiments, the cryoprotectant of the cryoprotectant solution comprises about 50% (weight/weight) sucrose. In some embodiments, the cryoprotectant of the cryoprotectant solution comprises about 40% to about 60% (weight/weight) dextran. In some embodiments, the cryoprotectant of the cryoprotectant solution comprises about 45% to about 55% (weight/weight) dextran. In some embodiments, the cryoprotectant of the cryoprotectant solution comprises about 50% (weight/weight) dextran. In some embodiments, the cryoprotectant of the cryoprotectant solution comprises about 40% to about 60% (weight/weight) sucrose and about 40% to about 60% (weight/weight) dextran. In some embodiments, the cryoprotectant of the cryoprotectant solution comprises about 45% to about 55% (weight/weight) sucrose and about 45% to about 55% (weight/weight) dextran. In some embodiments, the cryoprotectant of the cryoprotectant solution comprises about 50% (weight/weight) sucrose and about 50% (weight/weight) dextran. In some embodiments, the cryoprotectant of the cryoprotectant solution comprises about 0.25% to about 5% (weight/weight) L-cysteine HCl. In some embodiments, the cryoprotectant of the cryoprotectant solution comprises about 0.5% to about 2.5% (weight/weight) L-cysteine HCl. In some embodiments, the cryoprotectant of the cryoprotectant solution comprises about 0.75% to about 1.5% (weight/weight) L-cysteine HCl. In some embodiments, the cryoprotectant of the cryoprotectant solution comprises about 1% (weight/weight) L-cysteine HCl. In some embodiments, the cryoprotectant of the cryoprotectant solution provided herein comprises: (i) about 50% (weight/weight) sucrose; (ii) about 50% (weight/weight) dextran; and (iii) about 1% (weight/weight) L-cysteine HCl. In some embodiments, the cryoprotectant of the cryoprotectant solution provided herein comprises: (i) about 50% (weight/weight) sucrose; and (ii) about 50% (weight/weight) dextran.

Embodiment 20. A pharmaceutical agent that comprises bacteria, wherein the pharmaceutical agent maintains its stability (e.g., e.g., for three, six, twelve, eighteen and/or twenty-four months under long-term (2-8° C.) and/or accelerated (23-27° C. (optionally at 60% relative humidity (RH))) storage conditions, e.g., as determined by total cell count (TCC), e.g., as determined by Coulter counter and described herein).

Embodiment 21. The pharmaceutical agent of embodiment 20, wherein the water content of the pharmaceutical agent is between about 0.5% and about 9% (e.g., between about 1% and about 8%, between about 1% and about 6%, (e.g., about 1.7%, e.g., 1.8%, e.g., about 2%, e.g., about 2.2%, e.g., about 2.3%, e.g., about 2.4%, e.g., about 2.8%, e.g., about 2.9%, e.g., about 3%, e.g., about 3.1%, e.g., about 3.2%, e.g., about 3.3%, e.g., about 3.5%, e.g., about 3.6%, e.g., about 4%, e.g., about 4.5%, e.g., about 5%, e.g., about 5.3%, e.g., about 5.4%, or e.g., about 7.8%), e.g., as determined by the Karl-Fischer method for water content analysis provided in Ph. Eur. method 2.5.12).

Embodiment 22. The pharmaceutical agent of embodiment 20 or 21, wherein the pharmaceutical agent maintains its water content (e.g., e.g., for three, six, twelve, eighteen and/or twenty-four months under long-term (2-8° C.) and/or accelerated (25° C. (optionally at 60% RH)) storage conditions).

Embodiment 23. The pharmaceutical agent of any one of embodiments 20 to 22, wherein the pharmaceutical agent is of bacterial origin (e.g., mixture of selected strains or agents (e.g., components) or a single selected strain and/or agents (e.g., components); the pharmaceutical agent is a powder that comprises the bacteria and/or components thereof; comprises additional agents (e.g., cryoprotectant); and/or the pharmaceutical agent is a freeze dried (e.g., lyophilized) powder of bacteria and/or components thereof that optionally, further comprise additional agents (e.g., cryoprotectant).

Embodiment 24. A solid dosage form that comprises a pharmaceutical agent of any one of embodiments 1 to 8 or 20 to 23.

Embodiment 25. The solid dosage form of embodiment 24, wherein the solid dosage form is enteric coated; comprises a capsule; the capsule is a size 00, size 0, size 1, size 2, size 3, size 4, or size 5 capsule; the solid dosage form comprises a tablet (e.g., enterically coated tablet), optionally the tablet is a 5 mm, 6 mm, 7 mm, 8 mm, 9 mm, 10 mm, 11 mm, 12 mm, 13 mm, 14 mm, 15 mm, 16 mm, 17 mm, or 18 mm tablet; the solid dosage form comprises a minitablet, optionally the minitablet (e.g., enterically coated minitablet) is about a 1 mm minitablet to a 4 mm minitablet; a plurality of enterically coated minitablets are contained in a capsule (e.g., a size 0 capsule can contain about 31 to about 35 (e.g., 33) minitablets, wherein the minitablets are 3 mm in size), optionally the minitablets are 3 mm in size; and/or the capsule comprises HPMC (hydroxyl propyl methyl cellulose) or gelatin.

Embodiment 26. The solid dosage form of embodiment 24 or 25, wherein the enteric coating comprises one enteric coating; the enteric coating comprises an inner enteric coating and an outer enteric coating; the enteric coating comprises an inner enteric coating and an outer enteric coating, and wherein the inner and outer enteric coatings are not identical (e.g., the inner and outer enteric coatings do not contain identical components in identical amounts); the enteric coating (e.g., the one enteric coating or the inner enteric coating and/or the outer enteric coating) comprises a polymethacrylate-based copolymer; the enteric coating e.g., the one enteric coating or the inner enteric coating and/or the outer enteric coating) comprises a methacrylic acid ethyl acrylate (MAE) copolymer (1:1); the one enteric coating comprises methacrylic acid ethyl acrylate (MAE) copolymer (1:1); the one enteric coating comprises a Eudragit copolymer, e.g., a Eudragit, a Eudragit S, a Eudragit RL, a Eudragit RS, a Eudragit E, or a Eudragit FS; the enteric coating (e.g., the one enteric coating or the inner enteric coating and/or the outer enteric coating) comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate; or the enteric coating (e.g., the one enteric coating or the inner enteric coating and/or the outer enteric coating) comprises an anionic polymeric material.

Embodiment 27. The solid dosage form of any one of embodiments 24 to 26 or pharmaceutical agent of any one of embodiments 1 to 8 or 20 to 23, or pharmaceutical composition of embodiment 9 or the method of any one of embodiments 10 to 19, wherein the bacteria are of the genus Lactococcus, Prevotella, Bifidobacterium, or Veillonella.

Embodiment 28. The solid dosage form of any one of embodiments 24 to 26 or pharmaceutical agent of any one of embodiments 1 to 8 or 20 to 23, or pharmaceutical composition of embodiment 9 or the method of any one of embodiments 10 to 19, wherein the bacteria are of the species Lactococcus lactis cremoris, optionally the Lactococcus lactis cremoris is Lactococcus lactis cremoris Strain A (ATCC designation number PTA-125368); the bacteria are of the species Veillonella parvula, optionally the Veillonella parvula is Veillonella parvula (ATCC designation number PTA-125691); the bacteria are of the species Prevotella histicola, optionally the Prevotella histicola is Prevotella histicola Strain B 50329 (NRRL accession number B 50329); or the bacteria are of the species Bfidobacterium animalis, optionally the Bifidobacterium animalis is Bifidobacterium animalis ssp. lactis (ATCC designation number PTA-125097).

Embodiment 29. The solid dosage form of any one of embodiments 24 to 26 or pharmaceutical agent of any one of embodiments 1 to 8 or 20 to 23, or pharmaceutical composition of embodiment 9 or the method of any one of embodiments 10 to 19, wherein the freeze-dried powder comprises Prevotella bacteria or Veillonella bacteria; the bacteria are a species listed in Table 1, Table 2, Table 3, or Table 4, optionally the bacteria are a bacterial strain that has at least 95% genomic, 16S ribosomal ribonucleic acid, or clustered regularly interspaced short palindromic repeats sequence identity with a strain listed in Table 1 or Table 3; the bacterial are live, attenuated, or dead; the bacteria are of a taxonomic group listed in Table 1, Table 2, Table 3, or Table 4; the bacteria are a bacterial strain listed in Table 1, Table 2, Table 3, or Table 4; the bacteria are of a taxonomic group (e.g., class, order, family, genus, species or strain) listed in Table J; or the bacteria are a bacterial strain listed in Table J.

Embodiment 30. A method of preventing or treating a disease of a subject, the method comprising administering to the subject a solid dosage form of any one of embodiments 24 to 26.

Embodiment 31. Use of a pharmaceutical agent of any one of embodiments 1 to 8 or 20 to 23, or pharmaceutical composition of embodiment 9, or solid dosage form of any one of embodiments 24 to 26 for the treatment or prevention of a disease of a subject.

Embodiment 32. Use of a pharmaceutical agent of any one of embodiments 1 to 8 or 20 to 23, or pharmaceutical composition of embodiment 9, or solid dosage form of any one of embodiments 24 to 26 for the preparation of a medicament for treating or preventing a disease in a subject.

Embodiment 33. A pharmaceutical agent of any one of embodiments 1 to 8 or 20 to 23, or pharmaceutical composition of embodiment 9, or solid dosage form of any one of embodiments 24 to 26 for use in the treatment or prevention of a disease of a subject.

Embodiment 34. A cryoprotectant for use in preparing a pharmaceutical agent that comprises bacteria, optionally the cryoprotectant is a cryoprotectant solution.

Embodiment 35. A method of preparing a pharmaceutical agent, the method comprising combining bacteria with a cryoprotectant, optionally the cryoprotectant is a cryoprotectant solution, thereby preparing a formulated paste.

Embodiment 36. The method of embodiment 35, further comprising freeze drying the formulated paste, to thereby prepare a freeze-dried product; further comprising milling the freeze-dried product, to thereby prepare a freeze-dried powder; and/or further comprising combining the freeze-dried powder with one or more excipients to thereby prepare a pharmaceutical composition; wherein the cryoprotectant is mixed with the pellet in a ratio of 0.05 to 0.25 gram (g) cryoprotectant per gram of pellet (e.g., ratio of 0.15 to 0.2 gram (g) cryoprotectant solution per gram of pellet, ratio of about 0.18 gram (g) cryoprotectant solution per gram of pellet); or the cryoprotectant is mixed with the pellet at a ratio of 4% to 10% (volume/volume), (e.g., a ratio of about 5% to 8% (volume/volume), a ratio of about 6.5% (volume/volume)).

Embodiment 37. The cryoprotectant of embodiment 34 or the method of embodiment 35 or 36, wherein the cryoprotectant comprises sucrose; the cryoprotectant comprises dextran; the cryoprotectant comprises sucrose and dextran, optionally the cryoprotectant comprises sucrose and dextran in equivalent amounts; the cryoprotectant comprises sucrose, dextran, and L-cysteine HCl; or the cryoprotectant does not comprise L-cysteine HCl.

Embodiment 38. The cryoprotectant of embodiment 34 or the method of embodiment 35 or 36, wherein the cryoprotectant comprises about 10% to about 30% (weight/weight) sucrose (e.g. about 15% to about 35% (weight/weight) sucrose, about 20% (weight/weight) sucrose); the cryoprotectant comprises about 10% to about 30% (weight/weight) dextran (e.g., about 15% to about 35% (weight/weight) dextran, about 20% (weight/weight) dextran); the cryoprotectant comprises about 10% to about 30% (weight/weight) sucrose and about 10% to about 30% (weight/weight) dextran (e.g., about 15% to about 35% (weight/weight) sucrose and about 15% to about 35% (weight/weight) dextran, about 20% (weight/weight) sucrose and about 20% (weight/weight) dextran); the cryoprotectant comprises about 40% to about 80% (weight/weight) water (e.g., about 50% to about 70% (weight/weight) water, about 55% to about 65% (weight/weight) water, about 60% (weight/weight) water, about 59.8% (weight/weight) water, about 59.6% (weight/weight) water); the cryoprotectant comprises about 0.05% to about 0.6% (weight/weight) L-cysteine HCl (e.g., about 0.1% to about 0.5% (weight/weight) L-cysteine HCl, about 0.2% (weight/weight) L-cysteine HCl, about 0.4% (weight/weight) L-cysteine HCl); or the cryoprotectant comprises about 0.25% to about 5% (weight/weight) L-cysteine HCl. In some embodiments, the cryoprotectant provided herein comprises: (i) about 20% (weight/weight) sucrose; (ii) about 20% (weight/weight) dextran; (iii) about 0.4% (weight/weight) L-cysteine HCl; and (iv) about 59.6% (weight/weight) water. In some embodiments, the cryoprotectant provided herein comprises: (i) about 20% (weight/weight) sucrose; (ii) about 20% (weight/weight) dextran; (iii) about 0.2% (weight/weight) L-cysteine HCl; and (iv) about 59.8% (weight/weight) water. In some embodiments, the cryoprotectant provided herein comprises: (i) about 20% (weight/weight) sucrose; (ii) about 20% (weight/weight) dextran; and (iii) about 60% (weight/weight) water. In some embodiments, the cryoprotectant comprises about 40% to about 60% (weight/weight) sucrose. In some embodiments, the cryoprotectant comprises about 45% to about 55% (weight/weight) sucrose. In some embodiments, the cryoprotectant comprises about 50% (weight/weight) sucrose. In some embodiments, the cryoprotectant comprises about 40% to about 60% (weight/weight) dextran. In some embodiments, the cryoprotectant comprises about 45% to about 55% (weight/weight) dextran. In some embodiments, the cryoprotectant comprises about 50% (weight/weight) dextran. In some embodiments, the cryoprotectant comprises about 40% to about 60% (weight/weight) sucrose and about 40% to about 60% (weight/weight) dextran. In some embodiments, the cryoprotectant comprises about 45% to about 55% (weight/weight) sucrose and about 45% to about 55% (weight/weight) dextran. In some embodiments, the cryoprotectant comprises about 50% (weight/weight) sucrose and about 50% (weight/weight) dextran. In some embodiments, the cryoprotectant comprises about 0.25% to about 5% (weight/weight) L-cysteine HCl. In some embodiments, the cryoprotectant comprises about 0.5% to about 2.5% (weight/weight) L-cysteine HCl. In some embodiments, the cryoprotectant comprises about 0.75% to about 1.5% (weight/weight) L-cysteine HCl. In some embodiments, the cryoprotectant comprises about 1% (weight/weight) L-cysteine HCl. In some embodiments, the cryoprotectant comprises: (i) about 50% (weight/weight) sucrose; (ii) about 50% (weight/weight) dextran; and (iii) about 1% (weight/weight) L-cysteine HCl. In some embodiments, the cryoprotectant provided herein comprises: (i) about 50% (weight/weight) sucrose; and (ii) about 50% (weight/weight) dextran.

Embodiment 39. The cryoprotectant of embodiment 34 or the method of embodiment 35 or 36, wherein the cryoprotectant comprises a dried (dehydrated) maize glucose syrup (such as Glucidex), such as a dried maize glucose syrup with a dextrose equivalent (DE) of 21 (such as Glucidex 21), optionally wherein the cryoprotectant further comprises sucrose, optionally wherein the cryoprotectant further comprises dextrose (also referred to as glucose). Optionally wherein the cryoprotectant further comprises monosodium glutamate.

Embodiment 40. The cryoprotectant of embodiment 34 or the method of embodiment 35 or 36, wherein the cryoprotectant (e.g., dry composition not containing water) comprises about 27% to about 47% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21). In some embodiments, the cryoprotectant (e.g., dry composition not containing water) comprises about 27% to about 47% (weight/weight) sucrose. In some embodiments, the cryoprotectant comprises about 32% to about 42% (weight/weight) sucrose. In some embodiments, the cryoprotectant comprises about 37% (weight/weight) sucrose. In some embodiments, the cryoprotectant comprises about 8% to about 18% (weight/weight) dextrose. In some embodiments, the cryoprotectant comprises about 11% to about 15% (weight/weight) dextrose. In some embodiments, the cryoprotectant comprises about 13% (weight/weight) dextrose. In some embodiments, the cryoprotectant comprises about 8% to about 18% (weight/weight) monosodium glutamate. In some embodiments, the cryoprotectant comprises about 11% to about 15% (weight/weight) monosodium glutamate. In some embodiments, the cryoprotectant comprises about 13% (weight/weight) monosodium glutamate. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21) and about 27% to about 47% (weight/weight) sucrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21) and about 32% to about 42% (weight/weight) sucrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21) and about 37% (weight/weight) sucrose. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) dextrose. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; and about 8% to about 18% (weight/weight) monosodium glutamate. In some embodiments, the cryoprotectant comprises about 27% to about 47% (weight/weight) dried (dehydrated) maize glucose syrup (such as Glucidex), such as a dried maize glucose syrup with a dextrose equivalent (DE) of 21 (such as Glucidex 21); about 27% to about 47% (weight/weight) sucrose; about 8% to about 18% (weight/weight) dextrose; and about 8% to about 18% (weight/weight) monosodium glutamate. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) dextrose. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; and about 11% to about 15% (weight/weight) monosodium glutamate. In some embodiments, the cryoprotectant comprises about 32% to about 42% (weight/weight) dried (dehydrated) maize glucose syrup (such as Glucidex), such as a dried maize glucose syrup with a dextrose equivalent (DE) of 21 (such as Glucidex 21); about 32% to about 42% (weight/weight) sucrose; about 11% to about 15% (weight/weight) dextrose; and about 11% to about 15% (weight/weight) monosodium glutamate. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) dextrose. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; and about 13% (weight/weight) monosodium glutamate. In some embodiments, the cryoprotectant comprises about 37% (weight/weight) dried (dehydrated) maize glucose syrup (such as Glucidex), such as a dried maize glucose syrup with a dextrose equivalent (DE) of 21 (such as Glucidex 21); about 37% (weight/weight) sucrose; about 13% (weight/weight) dextrose; and about 13% (weight/weight) monosodium glutamate. In some embodiments, the cryoprotectant comprises about 0.05% to about 0.6% (weight/weight) L-cysteine HCl (e.g., a form of L-cysteine). In some embodiments, the cryoprotectant comprises about 0.1% to about 0.5% (weight/weight) L-cysteine HCl. In some embodiments, the cryoprotectant comprises about 0.1% to about 0.25% (weight/weight) L-cysteine HCl. In some embodiments, the cryoprotectant comprises about 0.2% (weight/weight) L-cysteine HCl. In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; (iv) about 13% (weight/weight) monosodium glutamate; and (v) about 0.2% (weight/weight) L-cysteine HCl. In certain embodiments, the cryoprotectant provided herein comprises: (i) about 37% (weight/weight) dried (dehydrated) maize glucose syrup (such as Glucidex), such as a dried maize glucose syrup with a dextrose equivalent (DE) of 21 (such as Glucidex 21); (ii) about 37% (weight/weight) sucrose; (iii) about 13% (weight/weight) dextrose; and (iv) about 13% (weight/weight) monosodium glutamate.

Embodiment 41. The method of any one of embodiments 10 to 19 or 36, wherein the freeze-dried powder comprises about 35% to about 75% (weight/weight) dried bacteria (e.g. about 40% to about 70% (weight/weight) dried bacteria).

Embodiment 42. The method of any one of embodiments 10 to 19, 36, or 41 wherein the bacteria comprise Prevotella bacteria or Veillonella bacteria.

### EXAMPLES

### Example 1: Prevotella histicola Pharmaceutical Agent

Pharmaceutical agent comprising Prevotella histicola Strain B was prepared as follows. Prevotella histicola Strain B has been deposited as Prevotella histicola Strain B (NRRL accession number B 50329).

Once a desired level of growth was achieved, centrifugation (using continuous centrifugation) was performed on fermentation broth that contained bacteria to prepare a pellet. A cryoprotectant solution was combined with the pellet to prepare a formulated paste. The components of the cryoprotectant solution are provided in Table 5A (Table 5B provides the percentages of the non-water (dry) components). The cryoprotectant solution was added to the concentrated cells (pellet) at a ratio of 6.5% (v/v) and mixed to give a formulated cell paste. The formulated cell paste was loaded onto disposable trays inside stainless-steel trays. The formulated paste was freeze dried to prepare a freeze-dried product. The freeze drying included primary drying (shelf temperature −5° C.) and secondary drying (shelf temperature 25° C.). The total drying time was at least 48 hours. The freeze-dried product was milled (e.g., using a milling machine) to prepare a freeze-dried powder, e.g., pharmaceutical agent. The pharmaceutical agent was collected into polyethylene bags. Each bag was thereafter sealed under vacuum and then placed in a metalized polyethylene bag, then stored at about 5° C.

TABLE 5A
Cryoprotectant solution components

| Components | Percent (w/w) |
|---|---|
| Sucrose | 20% |
| Dextran | 20% |
| L-Cysteine-HCL | 0.4% |
| Water | 59.6% |

TABLE 5B
Cryoprotectant components (dry)

| Components | Percent (w/w) |
|---|---|
| Sucrose | 49.5% |
| Dextran | 49.5% |
| L-Cysteine-HCL | 1.0% |

### Example 2: Properties of Pharmaceutical Agent Containing Prevotella histicola

Five batches of pharmaceutical agent comprising Prevotella histicola Strain B were prepared according to Example 1 and evaluated.

Batch 1: Upon evaluation, the pharmaceutical agent had a TCC of 2.4×10¹² cells/gram as determined by Coulter counter, and a water content of 1.7%, as determined by Karl Fisher (Ph. Eur. 2.5.12). The acceptable TCC value is set at >5×10¹¹ cells/gram.

Batch 2: Upon evaluation, the pharmaceutical agent had a TCC of 2×10¹² cells/gram as determined by Coulter counter, and a water content of 2.0%, as determined by Karl Fisher (Ph. Eur. 2.5.12). The acceptable TCC value is set at >5×10¹¹ cells/gram.

Batch 3: Upon evaluation, the pharmaceutical agent had a TCC of 2.1×10¹² cells/gram as determined by Coulter counter, and a water content of 3.0%, as determined by Karl Fisher (Ph. Eur. 2.5.12). The acceptable TCC value is set at >5×10¹¹ cells/gram.

Batch 4: Upon evaluation, the pharmaceutical agent had a TCC of 1.5×10¹² cells/gram as determined by Coulter counter, and a water content of 2.0%, as determined by Karl Fisher (Ph. Eur. 2.5.12). The acceptable TCC value is set at >5×10¹¹ cells/gram.

Batch 5: Upon evaluation, the pharmaceutical agent had a TCC of 1.3×10¹² cells/gram as determined by Coulter counter, and a water content of 4.0%, as determined by Karl Fisher (Ph. Eur. 2.5.12). The acceptable TCC value is set at >5×10¹¹ cells/gram.

### Example 3: Stability of Prevotella histicola Batch 1

Batch 1 of pharmaceutical agent comprising Prevotella histicola Strain B was prepared as described in Example 1, and evaluated as described in Example 2.

Stability study samples were packaged using a vacuum sealed polyethylene bag and then placed inside a secondary metalized polyethylene bag.

The stability of Batch 1 pharmaceutical agent was assessed.

### Content and Potency: Total Cell Count (TCC)/gram:

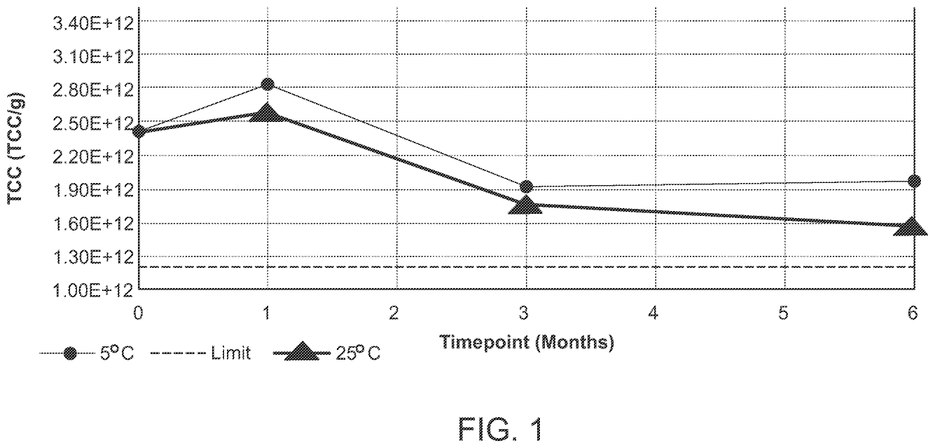

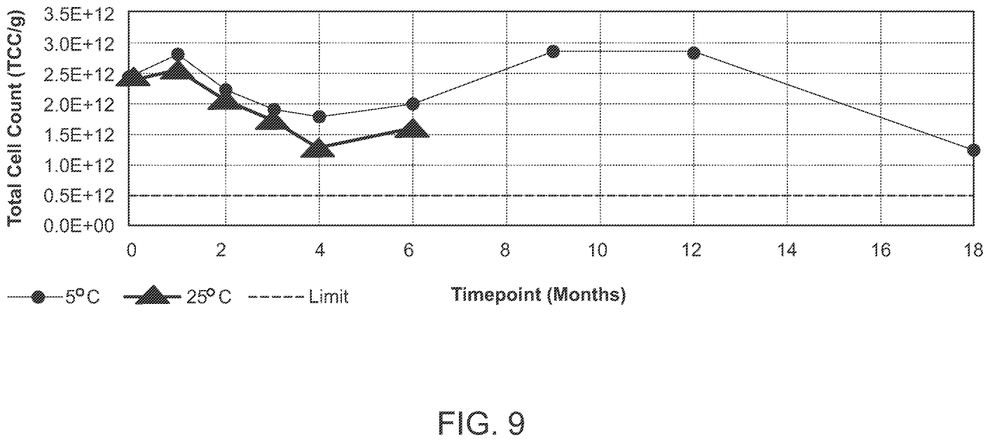

The total cells/gram was determined for the batch for the durations shown at long-term (2-8° C.) and accelerated (25° C.) storage conditions. TCC was determined by Coulter counter. The data are presented in FIG. 1. The data collected for up to 6 months showed stable total cell count profile with the analytical variability, and suggested that the product content and potency of pharmaceutical agent will be maintained during long term storage condition at 5° C. The data collected for up to 18 months are presented in FIG. 9.

### Water Content:

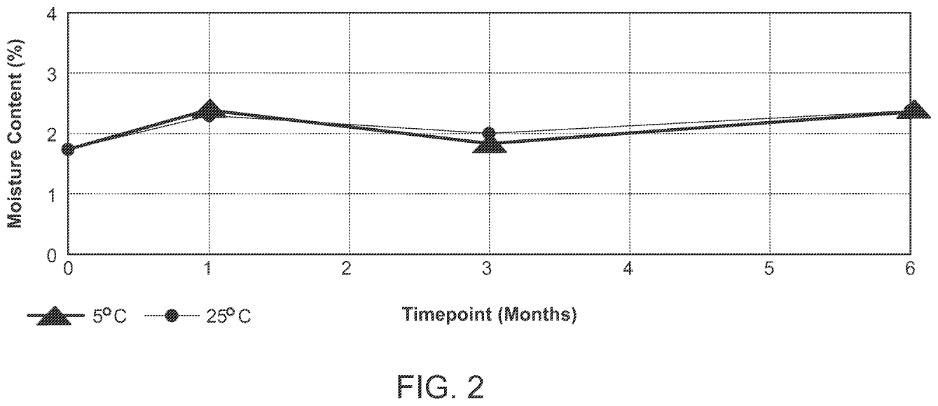

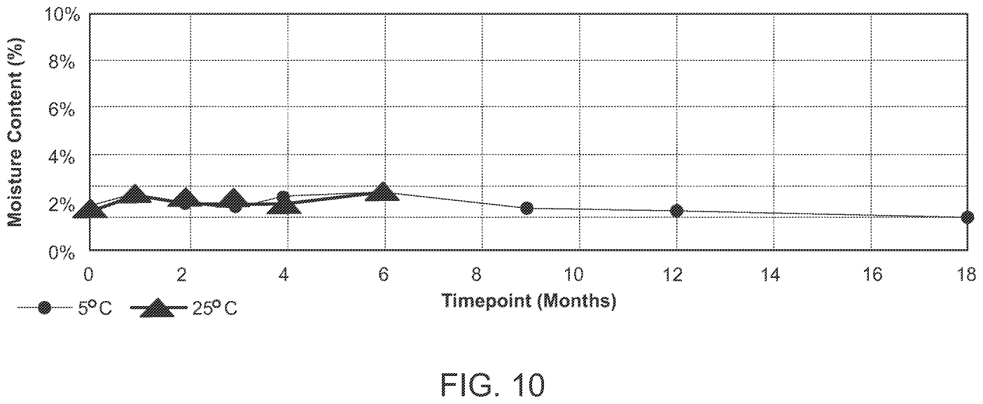

The water content was determined for the batch for the durations shown for both long-term (2-8° C.) and accelerated (25° C.) storage conditions for the durations shown. The results are shown in FIG. 2. No significant change in moisture content was observed for up to 6 months. The data collected for up to 18 months are presented in FIG. 10.

### Example 4: Stability of Prevotella histicola Batch 2

Batch 2 of pharmaceutical agent comprising Prevotella histicola Strain B was prepared as described in Example 1, and evaluated as described in Example 2.

Stability study samples were packaged using a vacuum sealed polyethylene bag and then placed inside a metalized polyethylene bag.

The stability of Batch 2 pharmaceutical agent was assessed.

### Content and Potency: Total Cell Count (TCC)/gram:

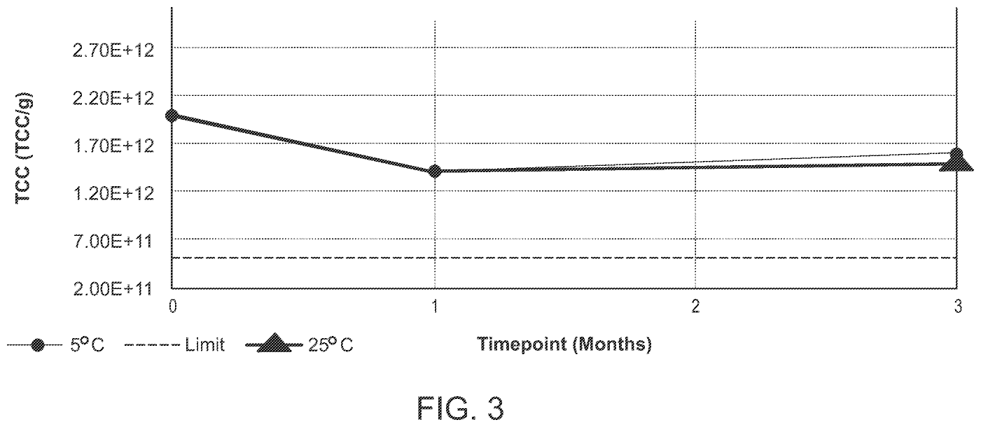

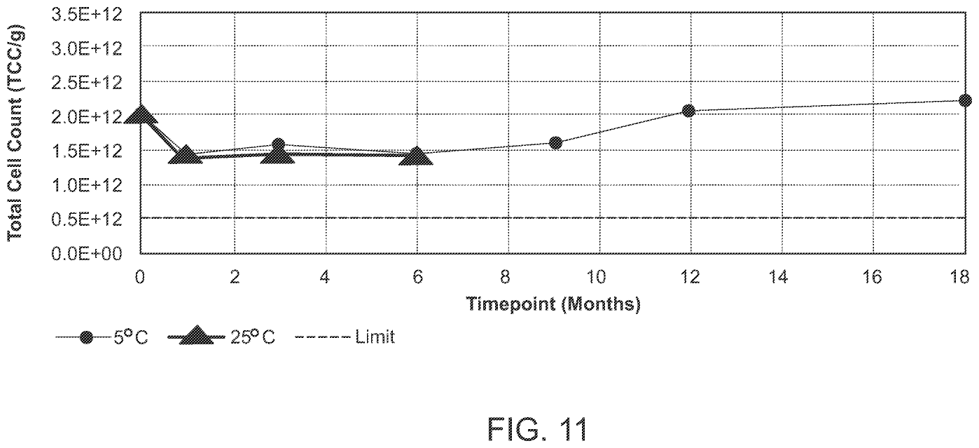

The total cells/gram was determined for the batch for the durations shown at long-term (2-8° C.) and accelerated (25° C.) storage conditions. TCC was determined by Coulter counter. The data are presented in FIG. 3. Total cell count stability profiles of the batch at 3 months showed no adverse trend. In addition, the total cell count values are comparable between the 5° C. (long-term) and 25° C. (accelerated) conditions for all the time points. These data provide support that the product content and potency will be maintained during storage. The data collected for up to 18 months are presented in FIG. 11.

### Water Content:

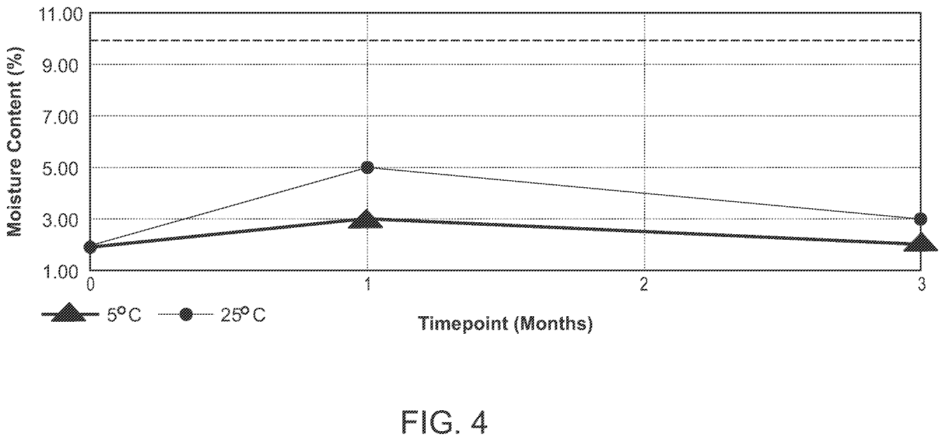

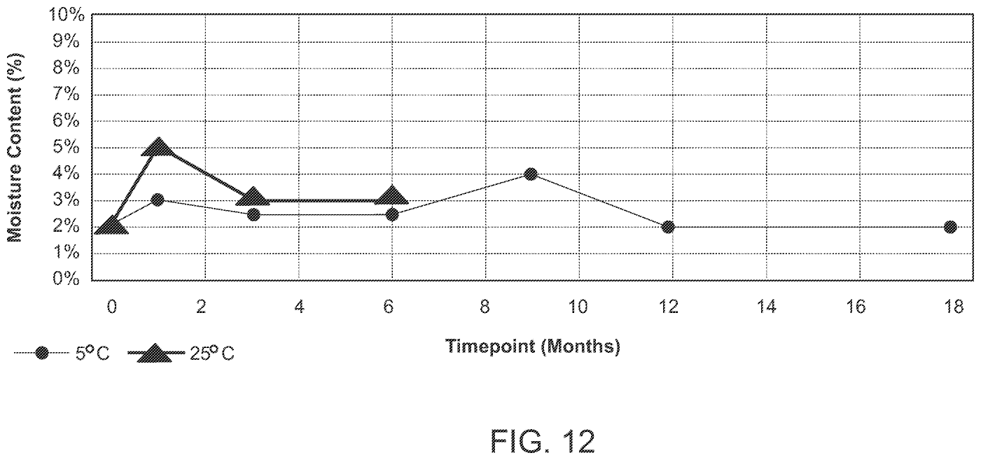

The water content was determined for the batch for the durations shown for both long-term (2-8° C.) and accelerated (25° C.) storage conditions for the durations shown. The results are shown in FIG. 4. No significant change in moisture content was observed for up to 3 months. The data collected for up to 18 months are presented in FIG. 12.

### Example 5: Veillonella parvula Pharmaceutical Agent

Pharmaceutical agent comprising Veillonella parvula Strain A was prepared as follows. Veillonella parvula Strain A has been deposited as Veillonella parvula Strain A (ATCC Deposit Number PTA-125691).

Once a desired level of growth was achieved, centrifugation (using continuous centrifugation) was performed on fermentation broth that contained bacteria to prepare a pellet. A cryoprotectant solution was combined with the pellet to prepare a formulated paste. The components of the cryoprotectant solution are provided in Table 6. The cryoprotectant was mixed with the cell pellet in a ratio of 0.18 g cryoprotectant per g of cell pellet to give a formulated cell paste. The formulated cell paste was loaded onto stainless-steel trays. The formulated paste was freeze dried to prepare a freeze-dried product. The freeze drying included primary drying (shelf temperature −5° C.) and secondary drying (shelf temperature 25° C.). The total drying time was at least 48 hours. The freeze-dried product was milled (e.g., using a milling machine) to prepare a freeze-dried powder, e.g., pharmaceutical agent. The pharmaceutical agent was collected into polyethylene bags. The polyethylene bags with approximately 0.5 kg of freeze-dried powder were vacuum sealed and placed inside a polyethylene bag with aluminum foil, then stored at about 5° C. The freeze-dried material in polyethylene bags with aluminum foil were transferred to boxes and gamma-irradiated within a dose rage of 20-35 kGy. After the gamma irradiation step pharmaceutical agent release assays were performed.

TABLE 6
Cryoprotectant solution components

| Components | Percent (w/w) |
|---|---|
| Sucrose | 20% |
| Dextran | 20% |
| L-Cysteine-HCL | 0.2% |
| Water | 59.8% |

### Example 6: Properties of Pharmaceutical Agent Containing Veillonella parvula

Six batches of pharmaceutical agent comprising Veillonella parvula Strain A were prepared according to Example 5 and evaluated.

Batch A: Upon evaluation, the pharmaceutical agent had a TCC of 2.4×10¹² cells/gram as determined by Coulter counter, and a water content of 3.2%, as determined by Karl Fisher (Ph. Eur. 2.5.12). The acceptable TCC value is set at ≥3.3×10¹¹ cells/gram.

Batch B: Upon evaluation, the pharmaceutical agent had a TCC of 1.5×10¹² cells/gram as determined by Coulter counter, and water content was not determined. The acceptable TCC value is set at ≥3.3×10¹¹ cells/gram.

Batch C: Upon evaluation, the pharmaceutical agent had a TCC of 2.2×10¹² cells/gram as determined by Coulter counter, and water content was not determined. The acceptable TCC value is set at ≥3.3×10¹¹ cells/gram.

Batch D: Upon evaluation, the pharmaceutical agent had a TCC of 12.1×10¹¹ cells/gram as determined by Coulter counter, and a water content of 2.2%, as determined by Karl Fisher (Ph. Eur. 2.5.12). The acceptable TCC value is set at ≥3.3×10¹¹ cells/gram.

Batch E: Upon evaluation, the pharmaceutical agent had a TCC of 7.7×10¹¹ cells/gram as determined by Coulter counter, and a water content of 3.0%, as determined by Karl Fisher (Ph. Eur. 2.5.12). The acceptable TCC value is set at ≥3.3×10¹¹ cells/gram.

Batch F: Upon evaluation, the pharmaceutical agent had a TCC of 11.4×10¹¹ cells/gram as determined by Coulter counter, and a water content of 2.8%, as determined by Karl Fisher (Ph. Eur. 2.5.12). The acceptable TCC value is set at ≥3.3×10¹¹ cells/gram.

### Example 7: Stability of Veillonella parvula Batch A

Batch A of pharmaceutical agent comprising Veillonella parvula Strain A was prepared as described in Example 5, and evaluated as described in Example 6.

Stability study samples were packaged using a polyethylene bag, vacuum sealed and then placed inside another polyethylene bag with aluminum foil.

The stability of Batch A pharmaceutical agent was assessed.

### Content and Potency: Total Cell Count (TCC)/Gram:

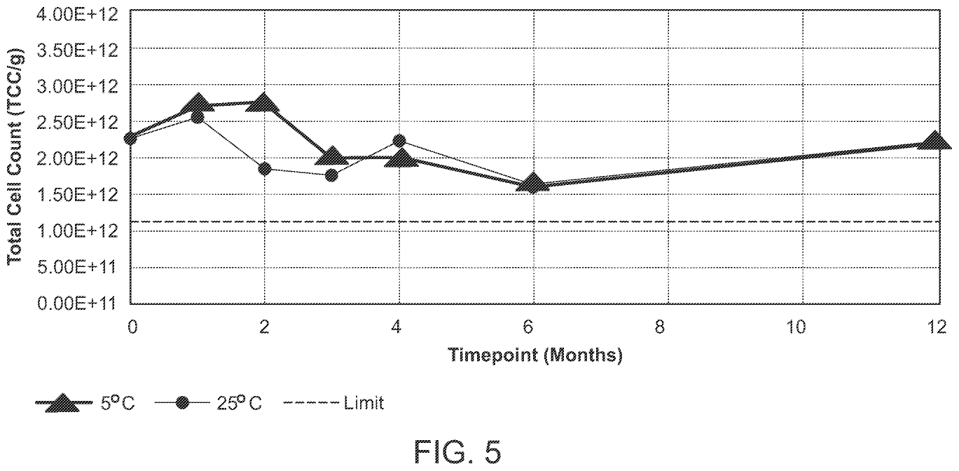

The total cells/gram was determined for the batch for the durations shown at long-term (2-8° C.) and accelerated (25° C. (±2° C.)/60% (±5%) RH (relative humidity)) (abbreviation: 25° C.)) storage conditions. TCC was determined by Coulter counter. The data are presented in FIG. 5. The data collected for up to 12 months showed stable total cell count profile for the batch at 5° C. and 25° C., which suggest that the product content and potency of the pharmaceutical agent will be maintained during long term storage conditions.

### Water Content:

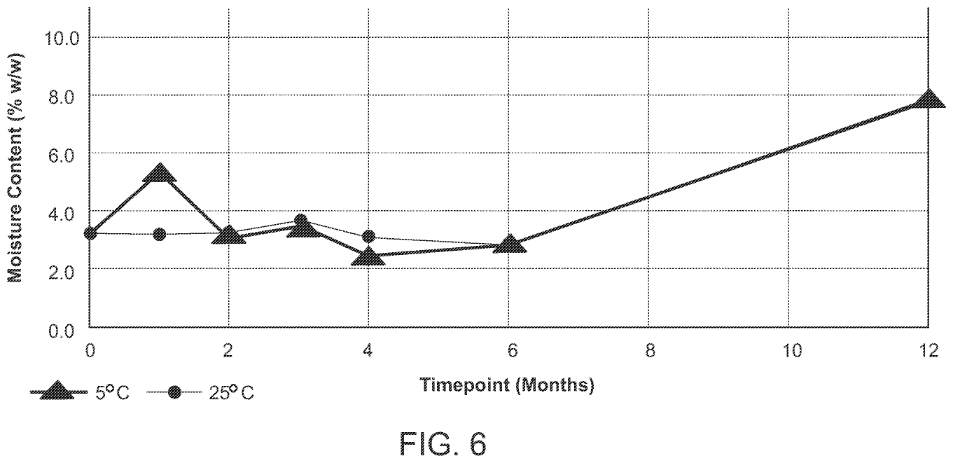

The water content was determined for the batch for the durations shown for both long-term (2-8° C.) and accelerated (25° C.) storage conditions for the durations shown. The results are shown in FIG. 6. No significant change in moisture content was observed for up to 12 months. With an initial upward trend at 1 month (5.0% to 5.4%), the moisture content remained unchanged at about 3% for up to 6 months. There was a slight increase in moisture content at 12 months, which could be due to normal variability.

### Example 8: Stability of Veillonella parvula Batch D

Batch D of pharmaceutical agent comprising Veillonella parvula Strain A was prepared as described in Example 5, and evaluated as described in Example 6.

Stability study samples were packaged using a polyethylene bag, vacuum sealed and then placed inside another polyethylene bag with aluminum foil.

The stability of Batch A pharmaceutical agent was assessed.

### Content and Potency: Total Cell Count (TCC)/Gram:

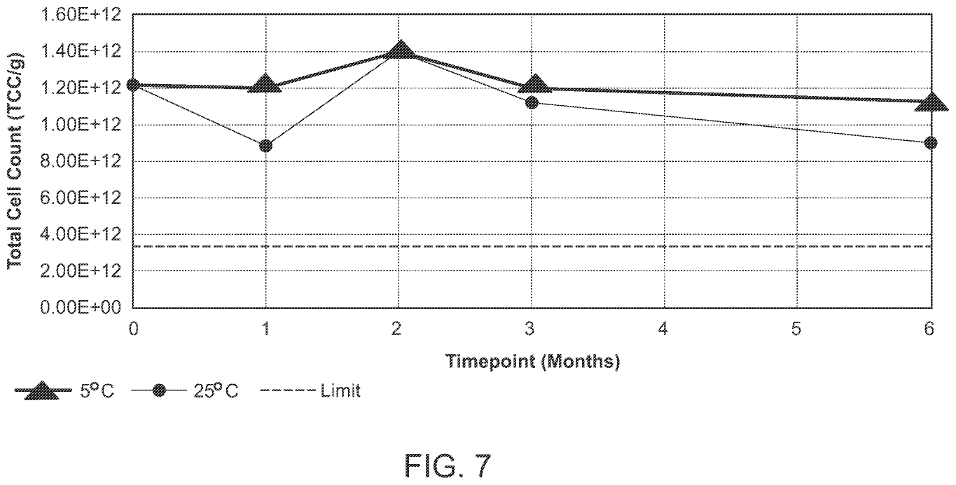

The total cells/gram was determined for the batch for the durations shown at long-term (2-8° C.) and accelerated (25° C. (±2° C.)/60% (±5%) RH (relative humidity)) (abbreviation: 25° C.)) storage conditions. TCC was determined by Coulter counter. The data are presented in FIG. 7. Total cell count stability profiles of the batch at 6 months showed no adverse trend. In addition, the total cell count values are comparable between the 5° C. (long-term) and 25° C. (accelerated) conditions for all the time points. These data provide support that the product content and potency will be maintained during storage.

### Water Content:

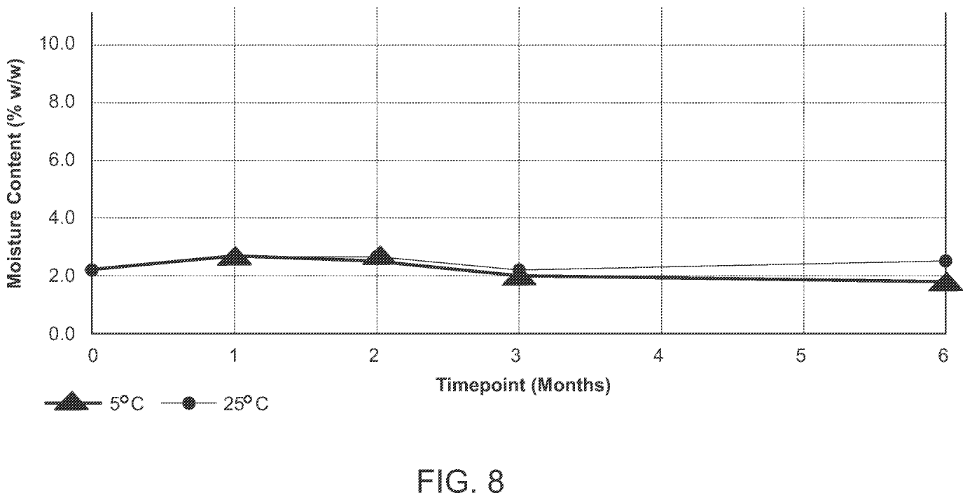

The water content was determined for the batch for the durations shown for both long-term (2-8° C.) and accelerated (25° C.) storage conditions for the durations shown. The results are shown in FIG. 8. No significant change in moisture content was observed for up to 12 months. No changes in moisture content was observed for the batch for up to 6 months.

### Example 9: Representative Strains

Bacteria of the taxonomic groups listed in Table J (e.g., class, order, family, genus, species or strain) can be used in the pharmaceutical agents described herein.

Information on the Gram staining, cell wall structure, and taxonomic classification for each strain is also provided in Table J.

TABLE J
Representative Strains

| | | Cell | | | | |
|---|---|---|---|---|---|---|
| | | envelope | | | | |
| Strain | Gram-stain | structure | Phylum | Class | Order | Family |
| Parabacteroides distasonis | Gram-stain-negative | diderm | Bacteroidota | Bacteroidia | Bacteroidales | Porphyromonadaceae |
| DRLU022118 A ILEUM-6 | | | | | | |
| Parabacteroides goldsteinii S4 | Gram-stain-negative | diderm | Bacteroidota | Bacteroidia | Bacteroidales | Porphyromonadaceae |
| Prevotella histicola | Gram-stain-negative | diderm | Bacteroidota | Bacteroidia | Bacteroidales | Prevotellaceae |
| Prevotella histicola | Gram-stain-negative | diderm | Bacteroidota | Bacteroidia | Bacteroidales | Prevotellaceae |
| Fournierella massiliensis S10 | Gram-stain-negative | monoderm | Firmicutes | Clostridia | Eubacteriales | Oscillospiraceae (formely |
| GIMucosa-297 | | | | | | Ruminococcaceae) |
| Harryflintia acetispora S4-M5 | Gram-stain-negative | monoderm | Firmicutes | Clostridia | Eubacteriales | Oscillospiraceae |
| Blautia massiliensis S1046-4A5 | Gram-stain-negative | monoderm | Firmicutes | Clostridia | Eubacteriales | Lachnospiraceae |
| Mediterraneibacter/ | Gram-stain-negative | monoderm | Firmicutes | Clostridia | Eubacteriales | Lachnospiraceae |
| [Ruminococcus] gnavus | | | | | | |
| S10 GIMucosa-412 | | | | | | |
| Clostridioides difficile S10 | Gram-stain-positive | monoderm | Firmicutes | Clostridia | Eubacteriales | Peptostreptococcaceae |
| GImucosa-525 | | | | | | |
| Aminipila sp. S16-M4 | Gram-stain-positive | monoderm | Firmicutes | Clostridia | Eubacteriales | Clostridiales Family |
| | | | | | | XIII/Incertae sedis 41/ |
| | | | | | | [Eubacteriales, no family] |
| Megasphaera sp. S29-N3 | Gram-stain-negative | diderm | Firmicutes | Negativicutes | Veillonellales | Veillonellaceae |
| Megasphaera sp. S1007 | Gram-stain-negative | diderm | Firmicutes | Negativicutes | Veillonellales | Veillonellaceae |
| Selenomonas felix S34N-300R | Gram-stain-negative | diderm | Firmicutes | Negativicutes | Selenomonadales | Selenomonadaceae |
| Veillonella parvula S14Ileum-201 | Gram-stain-negative | diderm | Firmicutes | Negativicutes | Veillonellales | Veillonellaceae |
| Propionispora sp. DSM100705- | Gram-stain-negative | diderm | Firmicutes | Negativicutes | Selenomonadales | Sporomusaceae |
| 1A | | | | | | |
| Rarimicrobium hominis S24RS2- | Gram-stain-negative | diderm | Synergistota | Synergistia | Synergistales | Synergistaceae |
| T2-5 | | | | | | |
| Cloacibacillus evryensis S29-M8 | Gram-stain-negative | diderm | Synergistota | Synergistia | Synergistales | Synergistaceae |
| Veillonella parvula S14-205 | Gram-stain-negative | diderm | Firmicutes | Negativicutes | Veillonellales | Veillonellaceae |
| Veillonella sp/dispar ECD01-DP- | Gram-stain-negative | diderm | Firmicutes | Negativicutes | Veillonellales | Veillonellaceae |
| 201 | | | | | | |
| Veillonella parvulaldispar | Gram-stain-negative | diderm | Firmicutes | Negativicutes | Veillonellales | Veillonellaceae |
| ECD01-DP-223 | | | | | | |
| Veillonella parvula S16 | Gram-stain-negative | diderm | Firmicutes | Negativicutes | Veillonellales | Veillonellaceae |
| GIMucosa-95 | | | | | | |

### Example 10: Pharmaceutical Agents

Pharmaceutical agents containing Prevotella histicola Strain B or Veillonella parvula Strain A were prepared using cryoprotectant solutions of the recipes provided in Table 5A (for Prevotella histicola Strain B) and Table 6 (for Veillonella parvula Strain A).

Models were run to estimate the amounts (weight/weight (gram/gram)) of dry cells, sucrose, dextran and L-cysteine HCl in batches of pharmaceutical agent (powder) containing Prevotella histicola Strain B. The calculated amounts are provided in Tables 7A and 7B. The amount of L-cysteine HCl includes not only the contribution from the cryoprotectant solution but also a contribution from cell culturing conditions.

TABLE 7A

| | Batch 1′ | Batch 2′ | Batch 3′ | Batch 4′ | Batch 5′ | Batch 6′ |
|---|---|---|---|---|---|---|
| Dry Cells | 64% | 58% | 47% | 60% | 54% | 43% |
| (Prevotella | | | | | | |
| histicola) | | | | | | |
| Sucrose | 11% | 10% | 8% | 12% | 11% | 9% |
| Dextran 40k | 11% | 10% | 8% | 12% | 11% | 9% |
| Cysteine-HCl | 0.22% | 0.20% | 0.17% | 0.24% | 0.22% | 0.19% |

TABLE 7B

| | Batch | Batch | Batch | Batch | Batch | Batch |
|---|---|---|---|---|---|---|
| | 7′ | 8′ | 9′ | 10′ | 11′ | 12′ |
| Dry Cells | 71% | 65% | 55% | 67% | 61% | 51% |
| (Prevotella | | | | | | |
| histicola) | | | | | | |
| Sucrose | 9% | 8% | 7% | 10% | 9% | 7% |
| Dextran | 9% | 8% | 7% | 10% | 9% | 7% |
| 40k | | | | | | |
| Cysteine- | 0.18% | 0.17% | 0.15% | 0.20% | 0.19% | 0.16% |
| HCl | | | | | | |

Models were run to estimate the amounts (weight/weight (gram/gram)) of dry cells, sucrose, dextran and L-cysteine HCl in batches of pharmaceutical agent (powder) containing Veillonella parvula Strain A. The calculated amounts are provided in Tables 8A and 8B. The amount of L-cysteine HCl includes not only the contribution from the cryoprotectant solution but also a contribution from cell culturing conditions.

TABLE 8A

| | Batch | Batch | Batch | Batch | Batch | Batch | Batch | Batch | Batch |
|---|---|---|---|---|---|---|---|---|---|
| | 1″ | 2″ | 3″ | 4″ | 5″ | 6″ | 7″ | 8″ | 9″ |
| Dry Cells | 66% | 63% | 55% | 59% | 56% | 48% | 49% | 46% | 38% |
| (Veillonella parvula) | | | | | | | | | |
| Sucrose | 13% | 12% | 10% | 15% | 14% | 12% | 19% | 17% | 14% |
| Dextran 40k | 13% | 12% | 10% | 15% | 14% | 12% | 19% | 17% | 14% |
| Cysteine-HCl | 0.17% | 0.20% | 0.24% | 0.21% | 0.23% | 0.28% | 0.26% | 0.28% | 0.33% |

TABLE 8B

| | Batch | Batch | Batch | Batch | Batch | Batch | Batch | Batch | Batch |
|---|---|---|---|---|---|---|---|---|---|
| | 10″ | 11″ | 12″ | 13″ | 14″ | 15″ | 16″ | 17″ | 18″ |
| Dry Cells | 65% | 61% | 54% | 58% | 54% | 47% | 48% | 44% | 37% |
| (Veillonella | | | | | | | | | |
| parvula) | | | | | | | | | |
| Sucrose | 13% | 12% | 11% | 15% | 14% | 12% | 19% | 18% | 15% |
| Dextran 40k | 13% | 12% | 11% | 15% | 14% | 12% | 19% | 18% | 15% |
| Cysteine-HCl | 0.18% | 0.20% | 0.25% | 0.22% | 0.24% | 0.28% | 0.27% | 0.29% | 0.34% |

### Example 11: Stability of Prevotella histicola Batch 4

Batch 4 of pharmaceutical agent comprising Prevotella histicola Strain B was prepared as described in Example 1, and evaluated as described in Example 2.

Stability study samples were packaged using a vacuum sealed polyethylene bag and then placed inside a secondary metalized polyethylene bag.

The stability of Batch 4 pharmaceutical agent was assessed.

### Content and Potency: Total Cell Count (TCC)/Gram:

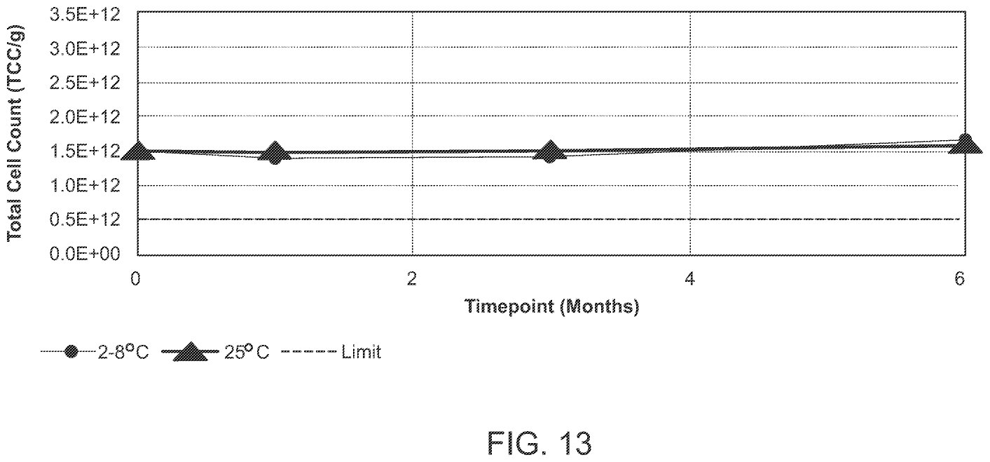

The total cells/gram was determined for the batch for the durations shown at long-term (2-8° C.) and accelerated (25° C.) storage conditions. TCC was determined by Coulter counter. The data are presented in FIG. 13.

### Water Content:

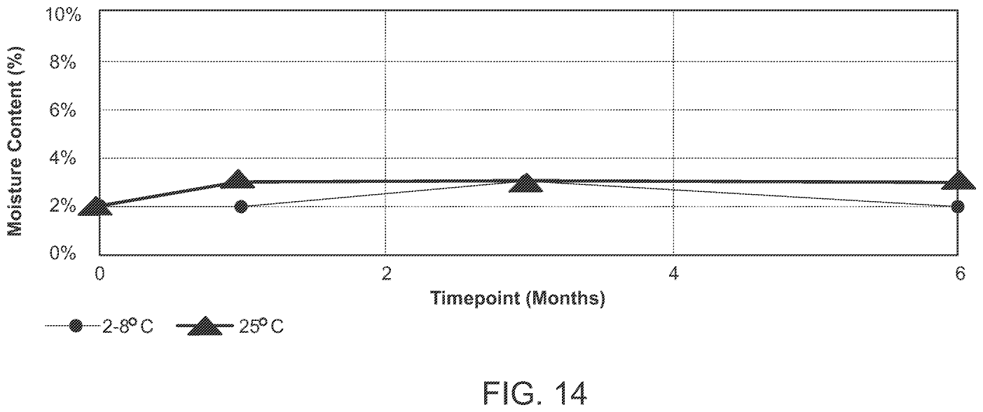

The water content was determined for the batch for the durations shown for both long-term (2-8° C.) and accelerated (25° C.) storage conditions for the durations shown. The results are shown in FIG. 14.

### Example 12: Stability of Prevotella histicola Batch 5

Batch 5 of pharmaceutical agent comprising Prevotella histicola Strain B was prepared as described in Example 1, and evaluated as described in Example 2.

Stability study samples were packaged using a vacuum sealed polyethylene bag and then placed inside a secondary metalized polyethylene bag.

The stability of Batch 5 pharmaceutical agent was assessed.

### Content and Potency: Total Cell Count (TCC)/Gram:

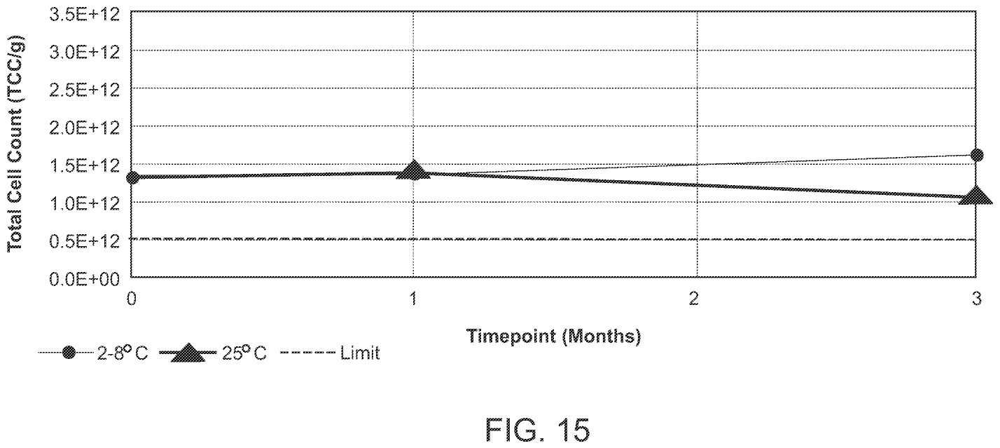

The total cells/gram was determined for the batch for the durations shown at long-term (2-8° C.) and accelerated (25° C.) storage conditions. TCC was determined by Coulter counter. The data are presented in FIG. 15.

### Water Content:

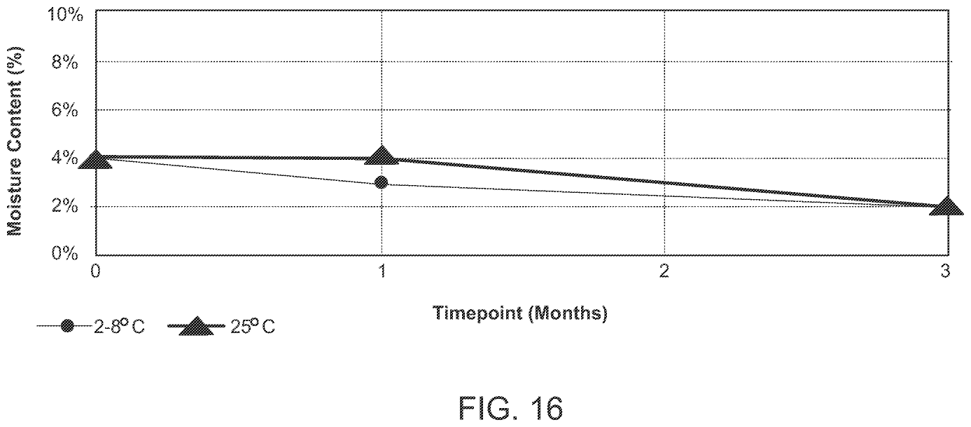

The water content was determined for the batch for the durations shown for both long-term (2-8° C.) and accelerated (25° C.) storage conditions for the durations shown. The results are shown in FIG. 16.

### Example 13: Prevotella histicola Pharmaceutical Agent

Pharmaceutical agent comprising Prevotella histicola Strain B was prepared as follows. Prevotella histicola Strain B has been deposited as Prevotella histicola Strain B (NRRL accession number B 50329).

Once a desired level of growth was achieved, centrifugation (using continuous centrifugation) was performed on fermentation broth that contained bacteria to prepare a pellet. A cryoprotectant solution was combined with the pellet to prepare a formulated paste. The components (percentages of the non-water (dry) components) of the cryoprotectant solution are provided in Table 9. The cryoprotectant solution was added to the concentrated cells (pellet) and mixed to give a formulated cell paste. The formulated cell paste was loaded onto disposable trays inside stainless-steel trays. The formulated paste was freeze dried to prepare a freeze-dried product. The freeze drying included primary drying (shelf temperature −20° C.) and secondary drying (shelf temperature 20° C.). The total drying time was at least 48 hours. The freeze-dried product was milled (e.g., using a milling machine) to prepare a freeze-dried powder, e.g., pharmaceutical agent. The pharmaceutical agent was collected into polyethylene bags. Each bag was thereafter sealed under vacuum and then placed in another polyethylene bag, then stored at about −20° C.

TABLE 9
Cryoprotectant components (dry)

| Components | Percent (w/w) |
|---|---|
| Glucidex 21 | 36.9% |
| Sucrose | 36.9% |
| Dextrose | 13.0% |
| Mono sodium Glutamate | 13.0% |
| L-Cysteine-HCl | 0.2% |

### Example 14: Properties of Pharmaceutical Agent Containing Prevotella histicola

A batch (Batch i) of pharmaceutical agent comprising Prevotella histicola Strain B was prepared according to Example 13 and evaluated.

Batch i: Upon evaluation, the pharmaceutical agent had a TCC of 5.9×10¹¹ cells/gram as determined by Coulter counter, and a water content of 4%, as determined by Karl Fisher (Ph. Eur. 2.5.12). The acceptable TCC value is set at ≥1×10¹¹ cells/gram.

### Example 15: Stability of Prevotella histicola Batch i

Batch i of pharmaceutical agent comprising Prevotella histicola Strain B was prepared as described in Example 13, and evaluated as described in Example 14.

Stability study samples were packaged using a vacuum sealed polyethylene bag and then placed inside another polyethylene bag.

The stability of Batch i pharmaceutical agent was assessed.

### Content and Potency: Total Cell Count (TCC)/Gram:

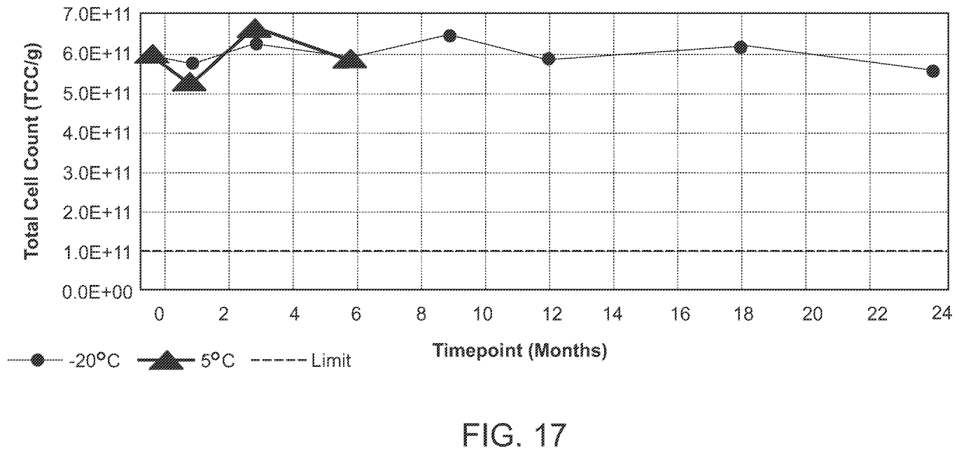

The total cells/gram was determined for the batch for the durations shown at long-term (−20° C.) and accelerated (2-8° C.) storage conditions. TCC was determined by Coulter counter. The data are presented in FIG. 17. The data collected for up to 24 months showed stable total cell count profile with the analytical variability, and suggested that the product content and potency of pharmaceutical agent will be maintained during long term storage condition at −20° C. The data suggest the pharmace4uetical agent is stable for 24 months at accelerated temp of 5 C.

### Water Content:

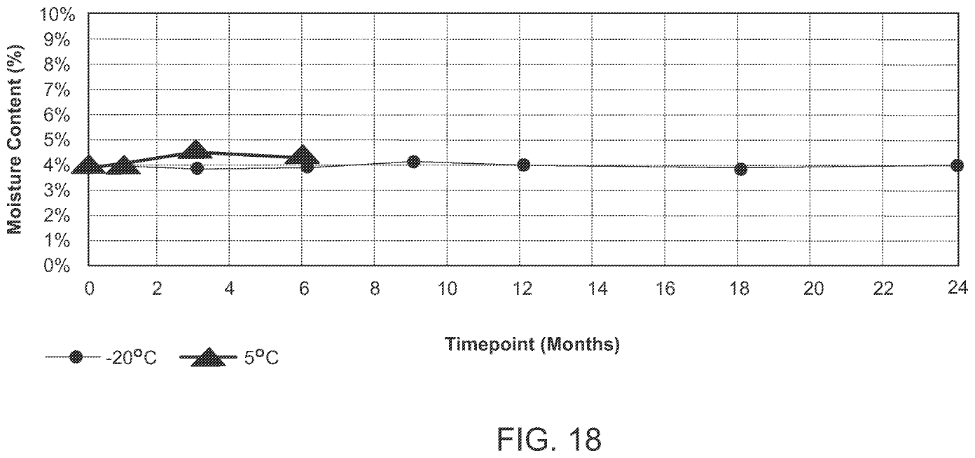

The water content was determined for the batch for the durations shown for both long-term (−20° C.) and accelerated (2-8° C.) storage conditions for the durations shown. The results are shown in FIG. 18. The moisture content remained steady at about 4% as observed at for up to 24 months at −20° C. and up to 6 months at 5° C.

### Example 16: Pharmaceutical Agents

Pharmaceutical agents containing Prevotella histicola Strain B were prepared using cryoprotectant solution of the recipe provided in Table 9.

Models were run to estimate the amounts (weight/weight (gram/gram)) of dry cells, Glucidex 21, sucrose, dextrose (also referred to as glucose), glutamate, and L-cysteine HCl in batches of pharmaceutical agent (powder) containing Prevotella histicola Strain B. The calculated amounts are provided in Table 10. The amount of L-cysteine HCl includes not only the contribution from the cryoprotectant solution but also a contribution from cell culturing conditions.

TABLE 10

| | Batch 1′′′ | Batch 2′′′ | Batch 3′′′ | Batch 4′′′ | Batch 5′′′ | Batch 6′′′ |
|---|---|---|---|---|---|---|
| Dry Cells | 28% | 25% | 22% | 23% | 22% | 19% |
| (Prevotella histicola) | | | | | | |
| Sucrose | 24% | 25% | 22% | 27% | 26% | 23% |
| Glucidex 21D | 24.7% | 26.2% | 25.0% | 27.9% | 27.3% | 25.9% |
| Glucose | 9.2% | 8.5% | 8.9% | 8.7% | 8.9% | 9.2% |
| Glutamate | 8.26% | 7.19% | 6.48% | 7.84% | 7.48% | 6.71% |
| Cysteine-HCl | 0.14% | 0.50% | 0.45% | 0.54% | 0.52% | 0.47% |

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

What is claimed is:

1. A pharmaceutical agent comprising bacteria, wherein the bacteria in the pharmaceutical agent are present at a total cell count (TCC) of at least 1×10¹¹ cells/gram of the pharmaceutical agent.

2. The pharmaceutical agent of claim 1, wherein the bacteria are present in the pharmaceutical agent at a total cell count (TCC) of from about 1×10¹¹ cells/gram to about 2.5×10¹² cells/gram of the pharmaceutical agent.

3. A pharmaceutical agent (e.g., powder) comprising bacteria and a cryoprotectant.

4. The pharmaceutical agent of claim 3, wherein the cryoprotectant comprises sucrose, dextran, or a combination thereof, optionally wherein the cryoprotectant comprises sucrose and dextran in equivalent amounts.

5. The pharmaceutical agent of claim 3, wherein the cryoprotectant comprises sucrose, dextran, and L-cysteine HCl.

6. The pharmaceutical agent of claim 3, wherein the cryoprotectant does not comprise L-cysteine HCl.

7. The pharmaceutical agent of claim 3, wherein the pharmaceutical agent comprises about 6% to about 12% (weight/weight) or about 7% to about 21% (weight/weight) sucrose;
about 6% to about 12% (weight/weight) or about 7% to about 21% (weight/weight) dextran; or
about 6% to about 12% (weight/weight) sucrose and about 6% to about 12% (weight/weight) dextran, or about 7% to about 21% (weight/weight) sucrose and about 7% to about 21% (weight/weight) dextran.

8. The pharmaceutical agent of claim 3, wherein the pharmaceutical agent comprises about 0.10% to about 0.25% (weight/weight) or about 0.15% to about 0.35% (weight/weight) L-cysteine HCl.

9. A pharmaceutical composition comprising a pharmaceutical agent of any one of claims 1 to 8 and one or more excipients.

10. A method of making a formulated paste comprising combining bacteria with a cryoprotectant, optionally wherein the cryoprotectant is a cryoprotectant solution, thereby preparing a formulated paste.

11. A method of preparing a pharmaceutical composition comprising:
(a) performing the method of claim 10 to make a formulated paste;
(b) freeze drying the formulated paste to prepare a freeze-dried product;
(c) milling the freeze-dried product to prepare a freeze-dried powder; and
(d) combining the freeze-dried powder with one or more excipients to prepare a pharmaceutical composition.

12. The method of claim 10 or 11, wherein the cryoprotectant is mixed with the pellet in a ratio of 0.1 to 0.25 gram (g) cryoprotectant per gram of pellet; or a ratio of 4% to 10% (volume/volume).

13. The method of any one of claims 10 to 12, wherein the cryoprotectant comprises sucrose, dextran, or a combination thereof, optionally wherein the cryoprotectant comprises sucrose and dextran in equivalent amounts.

14. The method of any one of claims 10 to 13, wherein the cryoprotectant comprises sucrose, dextran, and L-cysteine HCl.

15. The method of any one of claims 10 to 13, wherein the cryoprotectant does not comprise L-cysteine HCl.

16. The method of any one of claims 10 to 15, wherein the cryoprotectant comprises about 10% to about 30% (weight/weight) sucrose;
about 10% to about 30% (weight/weight) dextran; or
about 10% to about 30% (weight/weight) sucrose and about 10% to about 30% (weight/weight) dextran.

17. The method of any one of claims 10 to 16, wherein the cryoprotectant comprises about 40% to about 80% (weight/weight) water.

18. The method of any one of claims 10 to 14, wherein the cryoprotectant comprises about 0.05% to about 0.6% (weight/weight) L-cysteine HCl.

19. The method of any one of claims 10 to 14, wherein the cryoprotectant comprises about 0.25% to about 5% (weight/weight) L-cysteine HCl.

20. A pharmaceutical agent comprising bacteria, wherein the pharmaceutical agent maintains its stability.

21. The pharmaceutical agent of claim 20, wherein the water content of the pharmaceutical agent is between about 0.5% and about 9%.

22. The pharmaceutical agent of claim 20 or 21, wherein the pharmaceutical agent maintains its water content.

23. The pharmaceutical agent of any one of claims 20 to 22, wherein the pharmaceutical agent is of bacterial origin;
the pharmaceutical agent is a powder that comprises the bacteria and/or components thereof; comprises additional agents; and/or
the pharmaceutical agent is a freeze dried powder of bacteria and/or components thereof that optionally, further comprise additional agents.

24. A solid dosage form comprising a pharmaceutical agent of any one of claims 1 to 8 or 20 to 23.

25. The solid dosage form of claim 24, wherein the solid dosage form is enteric coated;
optionally wherein the solid dosage form comprises a capsule; optionally wherein the capsule is a size 00, size 0, size 1, size 2, size 3, size 4, or size 5 capsule, optionally wherein the capsule comprises HPMC (hydroxyl propyl methyl cellulose) or gelatin;
optionally wherein the solid dosage form comprises a tablet, optionally wherein the tablet is a 5 mm, 6 mm, 7 mm, 8 mm, 9 mm, 10 mm, 11 mm, 12 mm, 13 mm, 14 mm, 15 mm, 16 mm, 17 mm, or 18 mm tablet;
optionally wherein the solid dosage form comprises a minitablet, optionally wherein the minitablet is about a 1 mm minitablet to a 4 mm minitablet; and/or
optionally wherein the solid dosage form comprises a plurality of enterically coated minitablets contained in a capsule, optionally wherein the minitablets are 3 mm in size, optionally wherein the capsule comprises HPMC (hydroxyl propyl methyl cellulose) or gelatin.

26. The solid dosage form of claim 25, wherein:
the enteric coating comprises one enteric coating;
the enteric coating comprises an inner enteric coating and an outer enteric coating;
the enteric coating comprises an inner enteric coating and an outer enteric coating, and wherein the inner and outer enteric coatings are not identical;
the enteric coating comprises a polymethacrylate-based copolymer;
the enteric coating comprises a methacrylic acid ethyl acrylate (MAE) copolymer (1:1);
the one enteric coating comprises methacrylic acid ethyl acrylate (MAE) copolymer (1:1);
the one enteric coating comprises a Eudragit copolymer;
the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate; and/or
the enteric coating comprises an anionic polymeric material.

27. The solid dosage form of any one of claims 24 to 26 or pharmaceutical agent of any one of claims 1 to 8 or 20 to 23, or pharmaceutical composition of claim 9 or the method of any one of claims 10 to 19, wherein the bacteria are of the genus Lactococcus, Prevotella, Bifidobacterium, or Veillonella.

28. The solid dosage form of any one of claims 24 to 26 or pharmaceutical agent of any one of claims 1 to 8 or 20 to 23, or pharmaceutical composition of claim 9 or the method of any one of claims 10 to 19, wherein the bacteria are of the species Lactococcus lactis cremoris, optionally the Lactococcus lactis cremoris is Lactococcus lactis cremoris Strain A (ATCC designation number PTA-125368); the bacteria are of the species Veillonella parvula, optionally the Veillonella parvula is Veillonella parvula (ATCC designation number PTA-125691); the bacteria are of the species Prevotella histicola, optionally the Prevotella histicola is Prevotella histicola Strain B 50329 (NRRL accession number B 50329); or the bacteria are of the species Bifidobacterium animalis, optionally the Bifidobacterium animalis is Bifidobacterium animalis ssp. lactis (ATCC designation number PTA-125097).

29. The solid dosage form of any one of claims 24 to 26 or pharmaceutical agent of any one of claims 1 to 8 or 20 to 23, or pharmaceutical composition of claim 9 or the method of any one of claims 10 to 19, wherein the freeze-dried powder comprises Prevotella bacteria or Veillonella bacteria;
the bacteria are a species listed in Table 1, Table 2, Table 3, or Table 4, optionally the bacteria are a bacterial strain that has at least 95% genomic, 16S ribosomal ribonucleic acid, or clustered regularly interspaced short palindromic repeats sequence identity with a strain listed in Table 1 or Table 3;
the bacteria are of a taxonomic group listed in Table 1, Table 2, Table 3, or Table 4;
the bacteria are a bacterial strain listed in Table 1, Table 2, Table 3, or Table 4;
the bacteria are of a taxonomic group listed in Table J; and/or
the bacteria are a bacterial strain listed in Table J.

30. A method of preventing or treating a disease of a subject, the method comprising administering to the subject a solid dosage form of any one of claims 24 to 26.

31. Use of a pharmaceutical agent of any one of claims 1 to 8 or 20 to 23, or pharmaceutical composition of claim 9, or solid dosage form of any one of claims 24 to 26 for the treatment or prevention of a disease of a subject.

32. Use of a pharmaceutical agent of any one of claims 1 to 8 or 20 to 23, or pharmaceutical composition of claim 9, or solid dosage form of any one of claims 24 to 26 for the preparation of a medicament for treating or preventing a disease in a subject.

33. A pharmaceutical agent of any one of claims 1 to 8 or 20 to 23, or pharmaceutical composition of claim 9, or solid dosage form of any one of claims 24 to 26 for use in the treatment or prevention of a disease of a subject.

34. A cryoprotectant for use in preparing a pharmaceutical agent that comprises bacteria, optionally wherein the cryoprotectant is a cryoprotectant solution.

35. A method of preparing a pharmaceutical agent, the method comprising combining bacteria with a cryoprotectant, optionally wherein the cryoprotectant is a cryoprotectant solution, thereby preparing a formulated paste.

36. The cryoprotectant of claim 34 or the method of claim 35 or 36, wherein:
the cryoprotectant comprises sucrose;
the cryoprotectant comprises dextran;
the cryoprotectant comprises sucrose and dextran, optionally the cryoprotectant comprises sucrose and dextran in equivalent amounts;
the cryoprotectant comprises sucrose, dextran, and L-cysteine HCl; and/or
the cryoprotectant does not comprise L-cysteine HCl.
